(19) United States
(12) Patent Application Publication
(10) Pub. No.: US 2025/0228873 A1
(43) Pub. Date: Jul. 17, 2025

(54) C25 R AND S ISOMERS OF AMINOSTEROLS AND METHODS OF MAKING AND USING THE SAME

(71) Applicant: Enterin, Inc., Philadelphia, PA (US)

(72) Inventors: Denise BARBUT, Philadelphia, PA (US); Michael ZASLOFF, Philadelphia, PA (US); William A. KINNEY, Newtown, PA (US); Thorsten ROSNER, Berkeley Heights, NJ (US); Stephen R. JONES, King of Prussia, PA (US); Ian T. CROUCH, Metuchen, NJ (US)

(73) Assignee: Enterin, Inc., Philadelphia, PA (US)

(21) Appl. No.: 18/832,100

(22) PCT Filed: Jan. 24, 2023
(86) PCT No.: PCT/US2023/011414

§ 371 Date: Jul. 23, 2024

Related U.S. Application Data
(60) Provisional application No. 63/302,954, filed on Jan. 25, 2022.

(51) Int. Cl. A61K 31/58; A61K 31/575; A61P 25/28; C07J 41/00; C07J 71/00

(52) U.S. Cl. CPC A61K 31/58; A61K 31/575; A61P 25/28; C07J 41/0005; C07J 71/0026

(57) ABSTRACT

Compositions of C₂₅ stereodefined aminosterols and methods of making the same and using the same in therapeutic applications.

### CROSS-REFERENCE TO RELATED APPLICATION

The present application the benefit of U.S. Provisional Application No. 63/302,954, filed Jan. 25, 2022, the contents of which are incorporated herein by reference in their entirety.

### FIELD

The present application relates generally to aminosterol compounds for the treatment of human disease and methods of making the compounds.

### BACKGROUND OF THE INVENTION

Squalamine is the most abundant member of a larger aminosterol family comprising at least 12 related compounds (Rao et al., 2000).

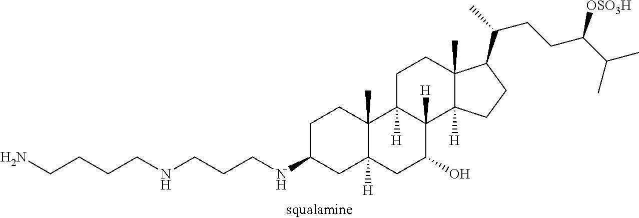

The discovery of squalamine, the structure of which is shown above, was reported by Michael Zasloff in 1993 (U.S. Pat. No. 5,192,756). Aminosterol 1436 is an aminosterol isolated from the dogfish shark, which is structurally related to squalamine (U.S. Pat. No. 5,840,936; Rao, Shinnar et al. 2000). It is also known as MSI-1436, trodusquemine and produlestan.

It is well understood that stereochemical purity of drugs is important in some therapeutics, single-enantiomer or compounds having stereodefinition at key carbon centers can provide greater selectivities for their biological targets, improved therapeutic indices, and/or better pharmacokinetics than a mixture of isomers. Thus, there is a need in the art to provide synthetic means for preparing C₂₅ stereodefined isomers of aminosterol compounds. This disclosure satisfies this need.

### SUMMARY

In one aspect, an aminosterol compound of Formula I-R is provided:

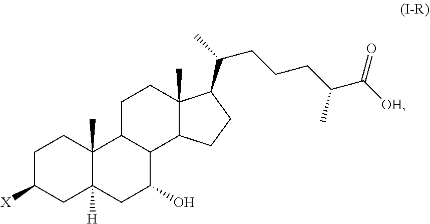

or a pharmaceutically acceptable salt, solvate, prodrug, or derivative thereof, wherein X is:

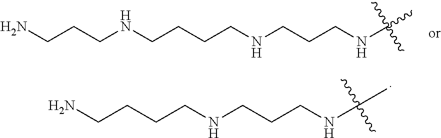 [SMILES: *NCCCNCCCCNCCCN ; *NCCCNCCCCN]

In one aspect, an aminosterol compound of Formula I-S is provided:

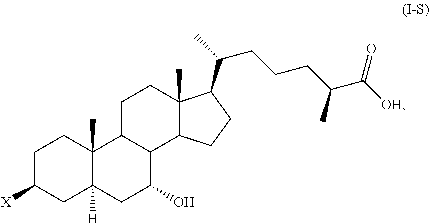

or a pharmaceutically acceptable salt, solvate, prodrug, or derivative thereof, wherein X is:

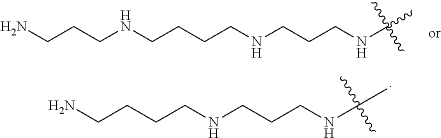 [SMILES: *NCCCNCCCCNCCCN ; *NCCCNCCCCN]

In one aspect, an aminosterol compound having the formula of Compound III-R is provided:

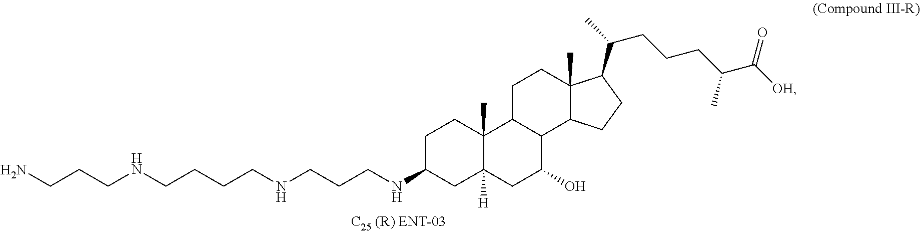 [SMILES: C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)O]

or a pharmaceutically acceptable salt, solvate, prodrug, or derivative thereof.

In one aspect, an aminosterol compound having the formula of Compound III-S is provided:

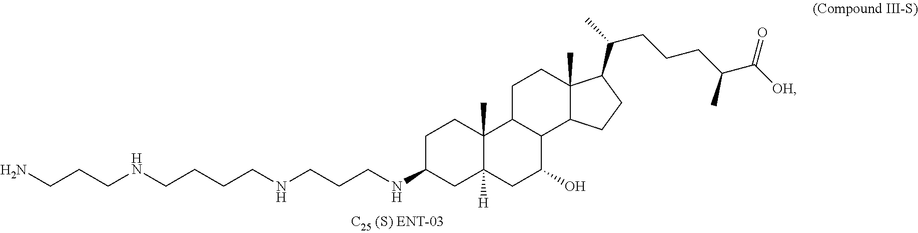 [SMILES: C[C@H](CCC[C@H](C)C(=O)O)[C@H]1CCC2C3C(CC[C@@]21C)[C@@]1(C)CC[C@H](NCCCNCCCCNCCCN)C[C@@H]1C[C@H]3O]

or a pharmaceutically acceptable salt, solvate, prodrug, or derivative thereof.

In one aspect, an aminosterol compound having the formula of Compound VI-R:

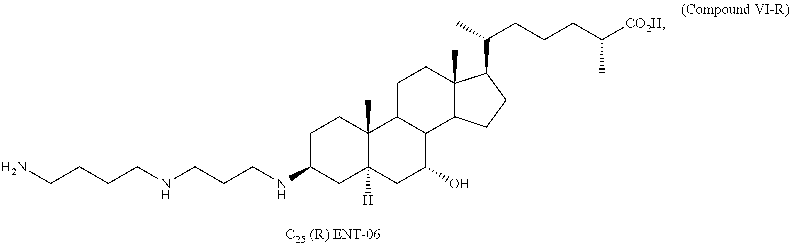 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]

or a pharmaceutically acceptable salt, solvate, prodrug, or derivative thereof, is provided.

In one aspect, an aminosterol compound having the formula of Compound VI-S:

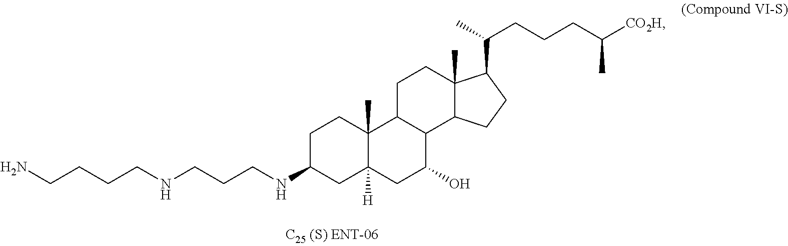 [SMILES: C[C@H](CCC[C@H](C)C(=O)O)[C@H]1CCC2C3C(CC[C@@]21C)[C@@]1(C)CC[C@H](NCCCNCCCCN)C[C@@H]1C[C@H]3O]

or a pharmaceutically acceptable salt, solvate, prodrug, or derivative thereof, is provided.

In one aspect, a compound having the formula of Compound I-R is provided:

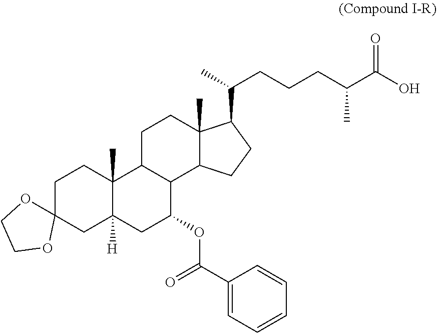 [SMILES: C[C@H](CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

or a pharmaceutically acceptable salt, solvate, or derivative thereof

In one aspect, a compound having the formula of Compound I-S is provided:

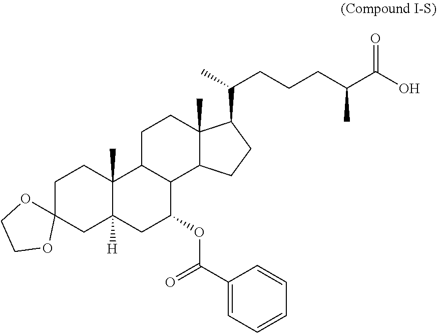 [SMILES: C[C@H](CCC[C@H](C)C(=O)O)[C@H]1CCC2C3C(CC[C@@]21C)[C@@]1(C)CCC2(C[C@@H]1C[C@H]3OC(=O)c1ccccc1)OCCO2]

or a pharmaceutically acceptable salt, solvate, or derivative thereof

In some embodiments, the aminosterol is a hydrochloride salt. In some embodiments, the aminosterol is a sodium salt.

In some embodiments, the diastereomeric excess (de), with respect to the C₂₅ carbon is about 80% to about 90%, about 90% to about 95%, about 95% to about 99%, about 99% to about 99.9%, or about 100%.

In one aspect, a composition is provided comprising an aminosterol compound disclosed herein, and at least one pharmaceutically acceptable carrier or excipient. In some embodiments, the composition comprises one or more of the following:(a) an aqueous carrier; (b) a buffer; (c) a sugar; and/or (d) a polyol compound.

In some embodiments, the composition further comprises at least one additional active agent. In some embodiments, the composition is formulated: (a) for administration selected from the group consisting of oral, pulmonary, rectal, colonic, parenteral, intracisternal, intravaginal, intraperitoneal, intravenous, subcutaneous, intramuscular, nebulization, inhalation, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

In some embodiments, the composition is formulated for oral administration. In some embodiments, the composition is formulated as an oral tablet or capsule. In some embodiments, the composition is formulated for intranasal administration.

In one aspect, a method of treating a subject in need having a condition susceptible to treatment with an aminosterol is provided, the method comprising administering to the subject a therapeutically effective amount of an aminosterol compound or a composition disclosed herein. In some embodiments, the condition is correlated with abnormal alpha-synuclein pathology and/or dopaminergic dysfunction.

In one aspect, a method of treating, preventing, and/or slowing the onset or progression of a condition or disorder, or a related symptom, correlated with abnormal alpha-synuclein pathology and/or dopaminergic dysfunction, in a subject in need is provided, the method comprising administering a therapeutically effective amount of an aminosterol compound or a composition disclosed herein.

In some embodiments: (a) the symptom is selected from the group consisting of constipation, hallucinations, cognitive impairment, and inflammation; (b) the symptom is correlated with a synucleopathy, a neurodegenerative disease, a neurological disease or disorder, a psychological and/or behavior disorder, or a cerebral or general ischemic disorder or condition; or (c) the condition or disorder is a synucleopathy, neurodegenerative disease, or neurological disease or disorder; (d) the condition or disorder is a psychological and/or behavior disorder; or (e) the condition or disorder is a cerebral or general ischemic disorder or condition.

In some embodiments: (a) the synucleopathy, neurodegenerative disease, or neurological disease or disorder is selected from the group consisting of Parkinson's disease, Alzheimer's disease, schizophrenia, multiple system atrophy, Lewy body dementia, dementia with Lewy bodies, Huntington's Disease, Multiple Sclerosis, Amyotrophic Lateral Sclerosis, Friedreich's ataxia, vascular dementia, spinal muscular atrophy, supranuclear palsy, progressive nuclear palsy, frontotemporal dementia, progressive nuclear palsy, Guadeloupian Parkinsonism, spinocerebellar ataxia, parkinsonism, traumatic brain injury, degenerative processes associated with aging, and dementia of aging; (b) the psychological or behavior disorder is selected from the group consisting of depression, autism, autism spectrum disorder, down syndrome, Gaucher's disease, Krabbe's disease, lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, and sleep disorders such as REM sleep behavior disorder (RBD), sleep fragmentation, REM behavior disorder, circadian rhythm dysfunction, sleep apnea, and cognitive impairment; or (c) the cerebral or general ischemic disorder or condition is selected from the group consisting of microangiopathy, intrapartum, cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects, high blood pressure, low blood pressure, and pulmonary edema.

In one aspect, a method of treating, preventing, and/or slowing the onset or progression a cerebral or general ischemic disorder and/or a related symptom, correlated with abnormal alpha-synuclein pathology and/or dopaminergic dysfunction, in a subject in need is provided, the method comprising administering a therapeutically effective amount of an aminosterol compound or a composition disclosed herein.

In some embodiments, the cerebral or general ischemic disorder and/or a related symptom is selected from the group consisting of microangiopathy, intrapartum cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, low blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects (CCDs) and/or a related symptom, and pulmonary edema.

In one aspect, a method of inhibiting one or more regulatory phosphatases in a subject is provided, the method comprising administering to the subject a therapeutically effective amount of an aminosterol compound or a composition disclosed herein.

In some embodiments, the regulatory phosphatase comprises protein Ser/Thr phosphatases including type 1 (PP1) and type 2 (PP2, i.e., PP2A, PP2C and PP2B) such as PPP1CA, PPP1CB, PPP1CC, PPP2CA, PPP2CB, PPP3CA, PPP3CB, PPP3CC, PPP4C PPP5C, and PPP6C; Class I Cys-based protein tyrosine phosphatases (PTPs); Class II Cys-based PTPs; Class III Cys-based PTPs; Class IV Cys-based DSPs (dual-specificity phosphatases); PTPs such as PTP1B, CDC14s (CDC14A, CDC14B, CDC14C, CDKN3); phosphatase and tensin homologs such as PTEN; slingshots such as SSH1, SSH2, and SSH3; dual specificity phosphatases such as DUSP1, DUSP2, DUSP3, DUSP4, DUSP5, DUSP6, DUSP7, DUSP8, DUSP9, DUSP10, DUSP11, DUSP12, DUSP13, DUSP14, DUSP15, DUSP16, DUSP18, DUSP19, DUSP21, DUSP22, DUSP23, DUSP26, DUSP27, and DUSP28; and other phosphatases such as CTDP1, CTDSP1, CTDSP2, CTDSPL, DULLARD, EPM2A, ILKAP, MDSP, PGAM5, PHLPP1, PHPLPP2, PPEF1, PPEF2, PPM1A, PPM1B, PPM1D, PPM1E, PPM1F, PPM1G, PPM1H, PPM1J, PPM1K, PPM1L, PPM1M, PPM1N, PPTC7, PTPMT1, SSU72, UBLCP1, PP1B, PP1A, PP2Aalpha/PP2R1A complex, PTPN6/SHP1, PTPRC/CD45, DUSP22/MKPX, PTPN2/TC-PTP, PTPN7/LC-PTP, PTPN12/PTP-PEST, PTPN1/PTP1B-CD, PTPN11/SHP2, PTPN11/SHP2-FL, and PTPN11/SHP2-FL(E76K). In some embodiments, the regulatory phosphatase comprises protein tyrosine phosphatase 1B (PTP1B).

In one aspect, a method of suppressing, preventing and/or slowing the onset or progression of appetite or weight gain, and/or one or more related symptoms, in a subject in need thereof is provided, the method comprising administering to the subject a therapeutically effective amount of an aminosterol compound or a composition disclosed herein.

In some embodiments (a) the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof; and/or (b) the method of administration is nasal administration, oral administration, or a combination thereof.

In some embodiments, the therapeutically effective amount of the aminosterol compound or a pharmaceutically acceptable salt, solvate, prodrug, or derivative thereof comprises: (a) about 0.1 to about 20 mg/kg body weight of the subject; (b) about 0.1 to about 15 mg/kg body weight of the subject; (c) about 0.1 to about 10 mg/kg body weight of the subject; (d) about 0.1 to about 5 mg/kg body weight of the subject; or (e) about 0.1 to about 2.5 mg/kg body weight of the subject.

In some embodiments, the therapeutically effective amount of the aminosterol compound or a pharmaceutically acceptable salt, solvate, prodrug, or derivative thereof comprises: (a) about 0.001 to about 500 mg/day; (b) about 0.001 to about 250 mg/day; (c) about 0.001 to about 125 mg/day; (d) about 0.001 to about 50 mg/day; (e) about 0.001 to about 25 mg/day; (f) about 0.001 to about 10 mg/day; (g) about 0.001 to about 6 mg/day; (h) about 0.001 to about 4 mg/day; or (i) about 0.001 to about 2 mg/day.

In some embodiments, the method of administration comprises oral administration and wherein the therapeutically effective amount of the aminosterol compound or a pharmaceutically acceptable salt, solvate, prodrug, or derivative thereof comprises: (a) about 1 to about 300 mg/day; or (b) about 25 to about 500 mg/day.

In some embodiments, the aminosterol compound or a pharmaceutically acceptable salt, solvate, prodrug, or derivative thereof is administered in combination with at least one additional active agent to achieve either an additive or synergistic effect.

In some embodiments, the additional active agent is administered via a method selected from the group consisting of: (a) concomitantly; (b) as an admixture; (c) separately and simultaneously or concurrently; and (d) separately and sequentially. In some embodiments, the additional active agent is a second aminosterol having a different structure from the aminosterol administered as described in the above paragraphs.

In some embodiments: (a) administration of the composition comprises administration on an empty stomach, optionally within two hours of the subject waking; and/or (b) no food is consumed by the subject after about 60 to about 90 minutes from administration of the composition.

In some embodiments, the aminosterol, or a pharmaceutically acceptable salt, solvate, prodrug, or derivative thereof, is of pharmaceutically acceptable grade.

In some embodiments, a phosphate salt of the aminosterol is administered. In some embodiments, a hydrochloride salt of the aminosterol is administered. In some embodiments, a sodium salt of the aminosterol is administered. In some embodiments, the subject is a human.

In some embodiments, the method further comprises (a) determining a dosage of the aminosterol or a pharmaceutically acceptable salt, solvate, prodrug, or derivative for the subject, wherein the aminosterol dosage is determined based on the effectiveness of the aminosterol dosage in improving or resolving a symptom being evaluated, (b) followed by administering a composition comprising the dosage of the aminosterol to the subject for a period of time, wherein the method comprises: (i) identifying a symptom to be evaluated, wherein the symptom is susceptible to treatment with an aminosterol; (ii) identifying a starting dosage of an aminosterol thereof for the subject; (iii) administering an escalating dosage of the aminosterol to the subject over a period of time until an effective dosage for the symptom being evaluated is identified, wherein the effective dosage is the aminosterol dosage where improvement or resolution of the symptom is observed, and fixing the aminosterol dosage at that level for that particular symptom in that particular subject.

In some embodiments, improvement or resolution of the symptom is measured using a clinically recognized scale or tool.

In some embodiments: (a) the composition is administered orally and: (i) the starting aminosterol dosage ranges from about 10 mg up to about 150 mg/day; (ii) the dosage of the aminosterol for the subject following escalation is fixed at a range of from about 25 mg up to about 500 mg/day; and/or (iii) the dosage of the aminosterol or a salt or derivative thereof is escalated in about 25 mg increments; or (b) the composition is administered intranasally and: (i) the starting aminosterol dosage ranges from about 0.001 mg to about 3 mg/day; (ii) the dosage of the aminosterol for the subject following escalation is fixed at a range of from about 0.001 mg up to about 6 mg/day; (iii) the dosage of the aminosterol for the subject following escalation is a dosage which is subtherapeutic when given orally or by injection; and/or (iv) the dosage of the aminosterol is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In some embodiments: (a) the dosage of the aminosterol is escalated every about 3 to about 5 days; and/or (b) the starting aminosterol dosage is higher if the symptom being evaluated is severe; and/or (c) the symptom is correlated with abnormal alpha-synuclein pathology and/or dopaminergic dysfunction.

In some embodiments, the symptom to be evaluated is selected from the group consisting of: (a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (1) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature; (t) improvement in breathing or apnea; (u) correction of cardiac conduction defect; (v) amelioration of pain; (w) restoration of bladder sensation and urination; (x) urinary incontinence; and/or (y) control of nocturia.

In some embodiments, the symptom to be evaluated is constipation, and wherein: (a) the fixed escalated aminosterol dosage for constipation is defined as the aminosterol dosage that results in a complete spontaneous bowel movement (CSBM) within 24 hours of dosing on at least 2 of 3 days at a given dosage; (b) if average complete spontaneous bowel movement (CSBM) or average spontaneous bowel movement (SBM) is greater than or equal to 1 per week, then the starting aminosterol dosage prior to escalation is 75 mg/day; and/or (c) if average CSBM or SBM is less than 1 per week, then the starting aminosterol dosage prior to escalation is 150 mg/day.

In one aspect, a method of increasing gene transcription in the gut of a subject is provided, the method comprising administering to the subject a therapeutically effective amount of an aminosterol compound or the composition disclosed herein.

In some embodiments, the increase in gene transcription is for one or more genes selected from the group consisting of caspase 14, collagen type XVII alpha 1, corneodesmosin, cornifelin, cystatin E/M, dermokine, desmocollin 1, desmoglein 1 beta, filaggrin, gap junction protein beta 4, gap junction protein beta 6, H19 imprinted maternally expressed transcript, hornerin, kallikrein related-peptidase 7 chymotryptic stratum, keratin 1, keratin 10, keratinocyte differentiation associated protein, keratinocyte expressed proline-rich, late cornified envelope 1A1, late cornified envelope 1A2, late cornified envelope 1, late cornified envelope 1C, late cornified envelope 1E, late cornified envelope 1F, late cornified envelope 1G, late cornified envelope 1H, late cornified envelope 11, late cornified envelope 1J, late cornified envelope 1L, late cornified envelope 1M, late cornified envelope 3C, late cornified envelope 3E, late cornified envelope 3F, lectin galactose binding soluble 7, loricrin, sciellin, myoglobin, myosin binding protein C slow-type, myosin heavy polypeptide 1 skeletal muscle, myosin heavy polypeptide 8 skeletal muscle, myosin light chain phosphorylatable fast ske, myosin light polypeptide 3, myozenin 1, myozenin 2, and titin-cap.

In some embodiments, the increase in gene transcription is selected from about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, about 250% to about 300%, about 300% to about 350%, about 350% to about 400%, about 400% to about 450%, about 500% to about 600%, about 600% to about 700%, about 700% to about 800%, about 800% to about 900%, about 900% to about 1000%, or about 1000% to about 1500%.

In one aspect, a method of inhibiting one or more regulatory phosphatases to achieve a therapeutic or prophylactic benefit is provided, the method comprising to a subject administering a therapeutically effective amount of an aminosterol compound or a composition disclosed herein.

In one aspect, a method of producing an aminosterol compound of Formula I-R, or a pharmaceutically acceptable salt, solvate, prodrug, or derivative thereof is provided:

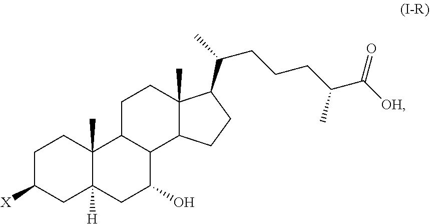

wherein X is

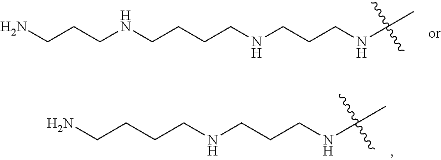 [SMILES: *NCCCNCCCCNCCCN ; *NCCCNCCCCN]

comprising asymmetric hydrogenation of Compound II:

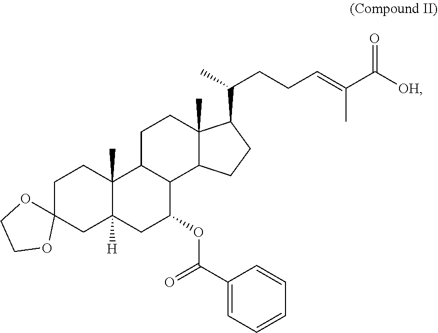 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

to produce Compound I-R:

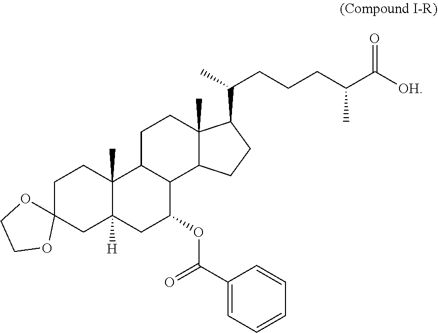 [SMILES: C[C@H](CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

In some embodiments, the compound of Formula I-R is of formula:

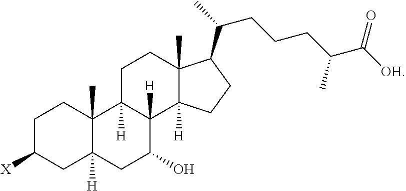

In one aspect, a method of producing an aminosterol compound of Formula I-S, or a pharmaceutically acceptable salt, solvate, prodrug, or derivative thereof is provided:

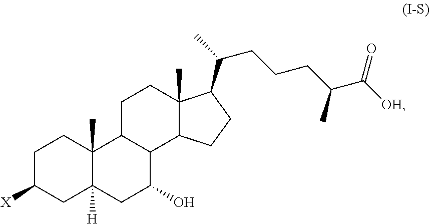

wherein X is

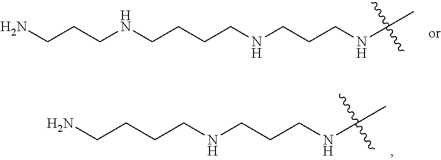 [SMILES: *NCCCNCCCCNCCCN ; *NCCCNCCCCN]

comprising asymmetric hydrogenation of Compound II:

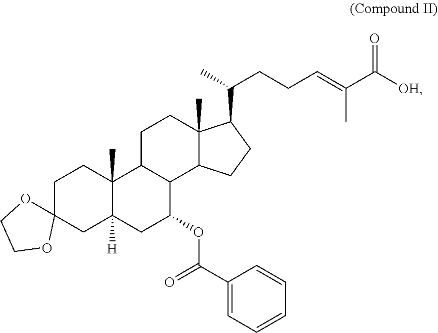 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

to produce Compound I-S:

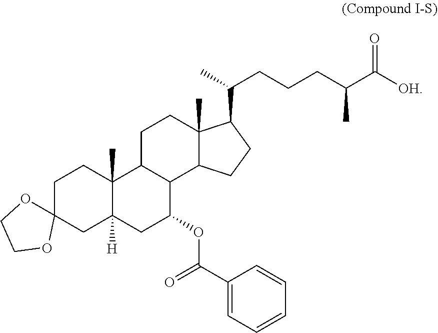 [SMILES: C[C@H](CCC[C@H](C)C(=O)O)[C@H]1CCC2C3C(CC[C@@]21C)[C@@]1(C)CCC2(C[C@@H]1C[C@H]3OC(=O)c1ccccc1)OCCO2]

In some embodiments, the compound of Formula I-S is of formula:

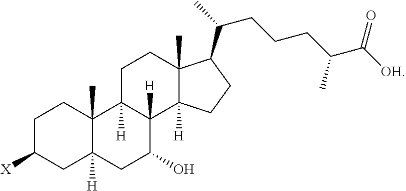

In some embodiments, Compound II is of formula:

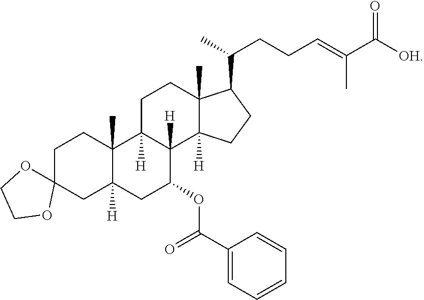

In some embodiments, X is

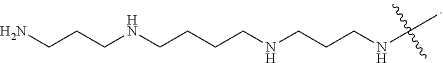 [SMILES: *NCCCNCCCCNCCCN]

In some embodiments, X is

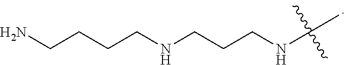 [SMILES: *NCCCNCCCCN]

In some embodiments, the method further comprises hydrolysis of a dioxolane of Compound I-S or Compound I-R to form a ketone and reductive amination of the ketone with amine:

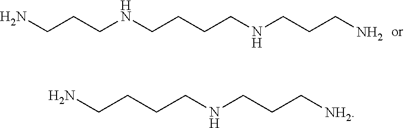 [SMILES: NCCCNCCCCNCCCN ; NCCCCNCCCN]

In one aspect, a method of producing an aminosterol compound having the formula of Compound III-R, or a pharmaceutically acceptable salt, solvate, prodrug, or derivative thereof, is provided:

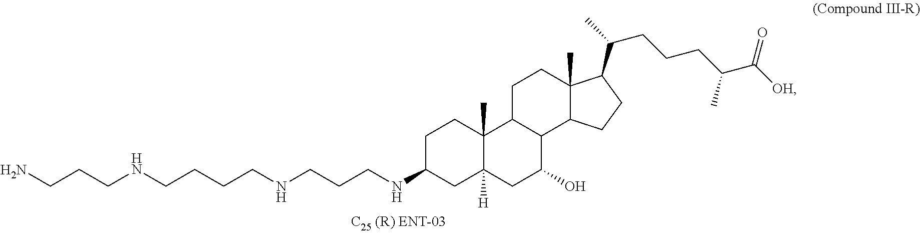 [SMILES: C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)O]

comprising asymmetric hydrogenation of Compound II:

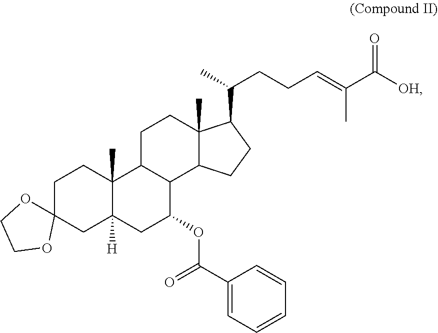 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

to produce Compound I-R:

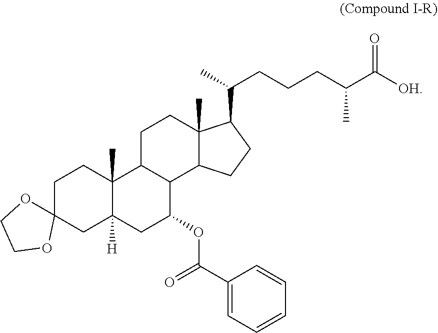 [SMILES: C[C@H](CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

In one aspect, a method of producing an aminosterol compound having the formula of Compound III-S, or a pharmaceutically acceptable salt, solvate, prodrug, or derivative thereof is provided:

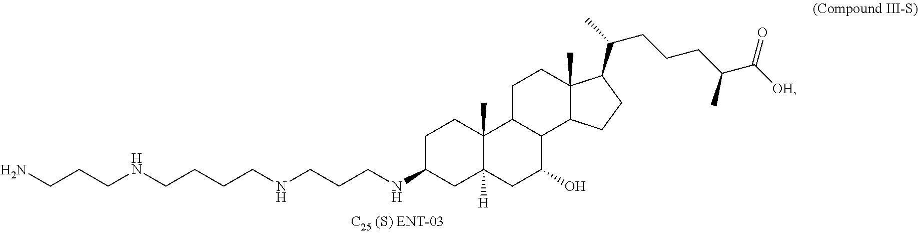 [SMILES: C[C@H](CCC[C@H](C)C(=O)O)[C@H]1CCC2C3C(CC[C@@]21C)[C@@]1(C)CC[C@H](NCCCNCCCCNCCCN)C[C@@H]1C[C@H]3O]

comprising asymmetric hydrogenation of Compound II:

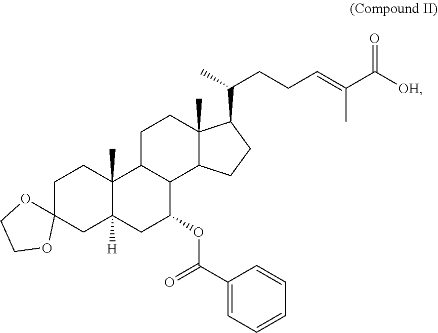 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

to produce Compound I-S:

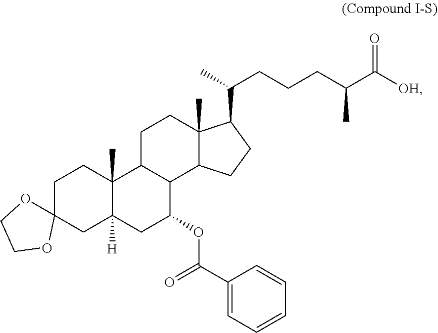 [SMILES: C[C@H](CCC[C@H](C)C(=O)O)[C@H]1CCC2C3C(CC[C@@]21C)[C@@]1(C)CCC2(C[C@@H]1C[C@H]3OC(=O)c1ccccc1)OCCO2]

In some embodiments, asymmetric hydrogenation comprises mixing Compound IV:

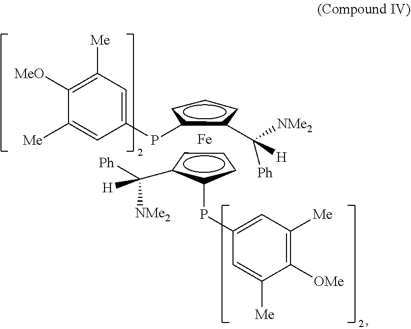

(Me-Allyl)2Ru(COD), and an acid to form a solution, and contacting Compound II with the solution and hydrogen (H₂).

In some embodiments, asymmetric hydrogenation comprises mixing Compound V:

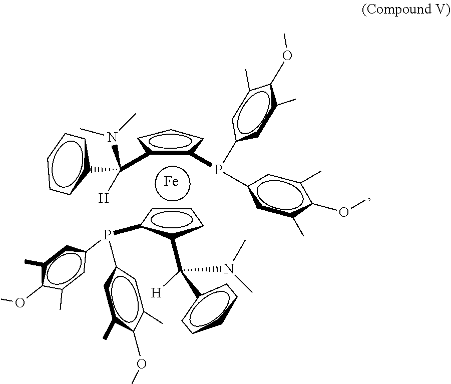

(Me-Allyl)2Ru(COD), and an acid to form a solution, and contacting Compound II with the solution and hydrogen (H₂).

In some embodiments, the acid comprises HBr. In some embodiments, the solution further comprises isopropyl acetate.

In some embodiments, the asymmetric hydrogenation comprises subjecting Compound II to about 60 psi to about 70 psi, about 70 psi to about 80 psi, about 80 psi to about 90 psi, about 90 psi to about 100 psi, about 100 psi to about 120 psi, about 120 psi to about 140 psi, about 140 psi to about 160 psi, about 160 psi to about 180 psi, about 180 psi to about 200 psi, about 200 psi to about 250 psi, about 250 psi to about 300 psi, about 300 psi to about 350 psi, about 350 psi to about 400 psi, about 400 psi to about 500 psi, about 500 psi to about 550 psi, about 550 psi to about 600 psi, about 600 psi to about 650 psi, or about greater than about 650 psi, of hydrogen (H₂).

In some embodiments, the asymmetric hydrogenation is at a temperature of about 20° C. to about 30° C., about 30° C. to about 40° C., about 40° C. to about 50° C., about 50° C. to about 60° C., or about greater than 60° C.

In some embodiments, the asymmetric hydrogenation is for about 12 hr to about 24 hr, about 24 hr to about 36 hr, about 36 hr to about 48 hr, about 48 hr to about 60 hr, or greater than about 60 hr.

In some embodiments: (a) the mol % s of Compound IV or Compound V, and (Me-Allyl)2Ru(COD) are about 0.2% to about 1%, about 1% to about 3%, about 3% to about 4%, about 4% to about 5%, about 5% to about 6%, or about 6% to about 8%, relative to Compound II; and (b) the mol % of the acid is about 4% to about 6%, about 6% to about 8%, about 8% to about 10%, about 10% to about 12%, or about 12% to about 16%, relative to Compound II.

In some embodiments, the diastereomeric excess (de) of the C₂₅ carbon is about 80% to about 90%, about 90% to about 95%, about 95% to about 99%, about 99% to about 99.9%, or about 100%.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIGS. 1A and 1B show percent weight loss over time in mice administered ENT-02 (MSI-1436) (circles) or ENT-03 (Compound III; squares). FIG. 1C shows intraperitoneal administration of ENT-03 to C57bl/6 male mice once weekly over 6 weeks caused a dose dependent weight loss.

FIG. 2 shows the results of administration of ENT-03 (Compound III) and ENT-02 (MSI-1436) on growing mice. While both compounds affected weight gain, ENT-02 had a more profound effect, having suppressed growth as well as having induced consumption of body fat. In contrast, the animals treated with ENT-03 continued normal growth, although they “slimmed down”, suggesting that ENT-03 re-established a new optimal body weight “set point.”

FIG. 3 shows images of a mucosal layer of the stomach showing a reduced mucosal layer in the 78 week old stomach (FIG. 3B) vs. the younger 20 week old stomach (FIG. 3A).

FIG. 4 shows IC₅₀ curves for PTP1B inhibition by three aminosterols tested ENT-02 (MSI-1436), ENT-03 (Compound III), and D-1436; FIG. 4A) and a control PTP1B inhibitor (FIG. 4B) according to Example 2.

FIGS. 5 A-H show that PTP1B inhibition by ENT-03 ameliorates the cognitive impairment of hAPP-J20 (FIGS. 5A-5D) and PS19 (FIGS. 5E-5H) mice.

FIGS. 6A-6C show volcano plots showing significance (as negative log₁₀-transformed FDR-adjusted p-values) against the magnitude (log 2-transformed fold change) of differentially expressed genes in the following three contrasts: (FIG. 6A) aged mice compared to young mice, (FIG. 6C) ENT-03-treated aged mice compared to vehicle-treated aged mice, (FIG. 6B) ENT-03-treated young mice compared to vehicle-treated young mice. Genes identified as having different levels between groups are represented as red (up-regulated) or blue (down-regulated) dots, and non-significant genes are represented as black dots. The horizontal red lines represent the applied p-value threshold.

FIGS. 7A and 7B show chromatograms for the LC/MS/MS analysis of brain extract from elderly humans for ENT-03. FIG. 7A is the chromatogram for the brain extract and FIG. 7B is a quality control sample of synthetic ENT-03.

FIGS. 8A and 8B show chromatograms for the LC/MS/MS analysis of mouse pup brain extract (FIG. 8A) and liver extract (FIG. 8B).

FIG. 9 shows approximate concentrations of ENT-03 measured in the brain and liver of neonatal mice over the first 3 weeks of life.

FIG. 10 shows gene expression profiles of the jejunum (FIGS. 10A-10D) or ileum (FIGS. 10D-10F) of young and aged mice treated with ENT-03 or control.

FIG. 11 shows a set of heatmaps investigating the overlap of differentially expressed genes between pairs of contrasts. The values in the plot represent the number of intersecting differentially expressed genes (adjusted p-value <0.05) between specific pairs of contrasts. The colors of the squares represent the Jaccard index (a quotient of the intersection and the union of genes) for the contrasts on the x- and y-axis: (FIG. 11A) differentially expressed genes in both directions, (FIG. 11B) up-regulated genes, (FIG. 11C) down-regulated genes, and (FIG. 11D) genes up-regulated in one contrast and down-regulated in the other contrast. Note that the numbers in grey squares represent the total number of selected differentially expressed genes of a specific contrast.

FIG. 12 shows the results on mouse weight gain following administration of ENT-03 as compared to administration of a deuterated form of ENT-03, ENT-03D₃.

FIGS. 13A and 13B show representative chromatograms of ENT-03 in 4 day old mouse brain extracts. FIG. 13A: MRM 619.6/545.5, upper tracing: endogenous ENT-03 in extract; MRM 623.6/549.5 lower tracing: extract+2.2 ng ENT-03-d₄/gram brain tissue; FIG. 13B: MRM 619.6/474.5, upper tracing: endogenous ENT-03 in extract; MRM 623.6/478.5 lower tracing: extract+2.2 ng ENT-03-d₄/gram brain tissue.

FIG. 14 shows a plot representing the weight loss results of male C57bl6/j (N=5/group) mice administered ENT-03 at varying dosages or vehicle by oral gavage every 3^{rd} day.

FIG. 15 shows a Venn diagram of transcripts down-regulated in ageing and up-regulated by ENT-03. A plot showing the numbers of overlapping and non-overlapping differentially expressed genes between the two sets of transcripts that were down-regulated in old versus young mice and up-regulated upon treatment compared to control. Numbers of features are shown from treatment with ENT-02 (MSI-1436) and ENT-03.

FIG. 16 shows a scatter plot comparing significant genes in ENT-02 (MSI-1436) vs control (young) against ENT-03 vs untreated (young). Genes are represented by points. The color of the point indicates which set the gene is assigned to. For each gene the log 2(fold change) in the ENT-02 (MSI-1436) vs control (young) contrast (y-axis) and the log 2(fold change) in the ENT-03 vs untreated (young) contrast (x-axis) are shown.

FIG. 17 shows an upset plot of significant genes according to the Examples. A plot showing the interaction between sets of up- and down-regulated genes. The leftmost barchart shows the size of each set used as input. The top barchart shows the exclusive size of each set (i.e., each gene is only counted once in this barchart). The dot-plot in the center shows the sets interacting in each case.

FIGS. 18A-18E: FIG. 18A: A Venn diagram of overlapping genes in MSI-1436 (aminosterol 1436) vs control (young) against ENT-03 vs untreated (young)—all vs all. FIG. 18B shows a Venn diagram of overlapping genes in MSI-1436 vs control (young) against ENT-03 vs untreated (young)—up vs up. FIG. 18C shows a Venn diagram of overlapping genes in MSI-1436 vs control (young) against ENT-03 vs untreated (young)—down vs down. FIG. 18D shows a Venn diagram of overlapping genes in MSI-1436 vs control (young) against ENT-03 vs untreated (young)—up vs down. FIG. 18E shows a Venn diagram of overlapping genes in MSI-1436 vs control (young) against ENT-03 vs untreated (young)—down vs up.

FIG. 19 shows a scatter plot comparing significant genes in MSI-1436 (aminosterol 1436) vs control (old) against ENT-03 vs untreated (old). Genes are represented by points. The color of the point indicates which set the gene is assigned to. For each gene the log 2(fold change) in the MSI-1436 vs control (old) contrast (y-axis) and the log 2(fold change) in the ENT-03 vs untreated (old) contrast (x-axis) are shown.

FIG. 20 shows an Upset plot of significant genes according to the Examples. A plot showing the interaction between sets of up- and down-regulated genes. The leftmost barchart shows the size of each set used as input. The top barchart shows the exclusive size of each set (i.e., each gene is only counted once in this barchart). The dot-plot in the center shows the sets interacting in each case.

FIGS. 21A-21E: FIG. 21A shows a Venn diagram of overlapping genes in ENT-02 (MSI-1436) vs control (old) against ENT-03 vs untreated (old)—all vs all. FIG. 21B shows a Venn diagram of overlapping genes in ENT-02 (MSI-1436) vs control (old) against ENT-03 vs untreated (old)—up vs up. FIG. 21C shows a Venn diagram of overlapping genes in ENT-02 (MSI-1436) vs control (old) against ENT-03 vs untreated (old)—down vs down. FIG. 21D shows a Venn diagram of overlapping genes in ENT-02 (MSI-1436) vs control (old) against ENT-03 vs untreated (old)—up vs down. FIG. 21E shows a Venn diagram of overlapping genes in ENT-02 (MSI-1436) vs control (old) against ENT-03 vs untreated (old)—down vs up.

FIG. 22 shows a scatter plot comparing significant genes in Old vs young (control) against Old vs young (untreated). Genes are represented by points. The color of the point indicates which set the gene is assigned to. For each gene the log 2(fold change) in the Old vs young (control) contrast (y-axis) and the log 2(fold change) in the Old vs young (untreated) contrast (x-axis) are shown.

FIG. 23 shows a Upset plot of significant genes according to the Examples. A plot showing the interaction between sets of up- and down-regulated genes. The leftmost barchart shows the size of each set used as input. The top barchart shows the exclusive size of each set (i.e., each gene is only counted once in this barchart). The dot-plot in the center shows the sets interacting in each case.

FIGS. 24A-24E: FIG. 24A shows a Venn diagram of overlapping genes in Old vs young (control) against Old vs young (untreated)—all vs all. FIG. 24B shows a Venn diagram of overlapping genes in Old vs young (control) against Old vs young (untreated)—up vs up. FIG. 24C shows a Venn diagram of overlapping genes in Old vs young (control) against Old vs young (untreated)—down vs down. FIG. 24D shows a Venn diagram of overlapping genes in Old vs young (control) against Old vs young (untreated)—up vs down. FIG. 24E shows a Venn diagram of overlapping genes in Old vs young (control) against Old vs young (untreated)—down vs up.

FIG. 25 shows a scatter plot comparing significant genes in Old vs young (ENT-02; MSI-1436) against Old vs young (ENT-03). Genes are represented by points. The color of the point indicates which set the gene is assigned to. For each gene the log 2(fold change) in the Old vs young (ENT-02; MSI-1436) contrast (y-axis) and the log 2(fold change) in the Old vs young (ENT-03) contrast (x-axis) are shown.

FIG. 26 shows an Upset plot of significant genes according to the Examples. A plot showing the interaction between sets of up- and down-regulated genes. The leftmost barchart shows the size of each set used as input. The top barchart shows the exclusive size of each set (i.e., each gene is only counted once in this barchart). The dot-plot in the center shows the sets interacting in each case.

FIGS. 27A-27E: FIG. 27A shows a Venn diagram of overlapping genes in Old vs young (MSI-1436) against Old vs young (ENT-03) all vs all. FIG. 27B shows a Venn diagram of overlapping genes in Old vs young (MSI-1436) against Old vs young (ENT-03)—up vs up.

FIG. 27C shows a Venn diagram of overlapping genes in Old vs young (MSI-1436) against Old vs young (ENT-03)—down vs down. FIG. 27D shows a Venn diagram of overlapping genes in Old vs young (MSI-1436) against Old vs young (ENT-03)—up vs down. FIG. 27E shows a Venn diagram of overlapping genes in Old vs young (MSI-1436) against Old vs young (ENT-03)—down vs up.

FIG. 28: Heatmaps of overlaps between contrasts: A plot showing the number of overlapping selected genes between the contrasts performed. Note that the numbers on the diagonal represent the total number of selected genes found for each contrast. The colors of the squares represent the Jaccard index (the intersection over the union) for the contrasts on the x-axis with those on the y-axis. FIG. 28A: Heatmap of overlaps of up- and down-regulated (y-axis) vs. up- and down-regulated (x-axis) selected genes for each contrast. FIG. 28B: Heatmap of overlaps of up-regulated (y-axis) vs. up-regulated (x-axis) selected genes for each contrast. FIG. 28C: Heatmap of overlaps of down-regulated (y-axis) vs. down-regulated (x-axis) selected genes for each contrast. FIG. 28D: Heatmap of overlaps of up-regulated (y-axis) vs. down-regulated (x-axis) selected genes for each contrast.

FIG. 29 shows a crystal structure of compound R-(24).

FIG. 30 shows an HPLC chromatogram highlighting the separation of the C₂₅ R and S isomers of ENT-03

### DETAILED DESCRIPTION

### I. Overview of the Invention

This disclosure is directed to compounds of Formula I:

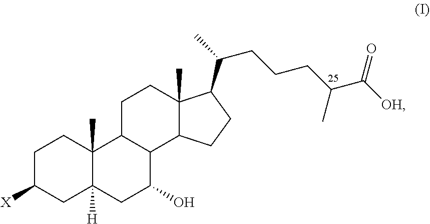

or a pharmaceutically acceptable salt, solvate, prodrug, or derivative thereof, and compositions comprising the same, wherein X is:

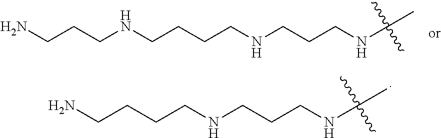 [SMILES: *NCCCNCCCCNCCCN ; *NCCCNCCCCN]

wherein the C₂₅ stereocenter (marked in Formula I with “25”) is enriched for either the R or S configuration. The disclosure is also directed to methods of making C₂₅ stereodefined compounds of Formula I and methods of using the compounds therapeutically.

Recently, a human derived derivative of aminosterol 1436, Compound III (ENT-03 or Hu-1436) was described, as shown below. See PCT/US2020/044390, filed Aug. 2, 2019.

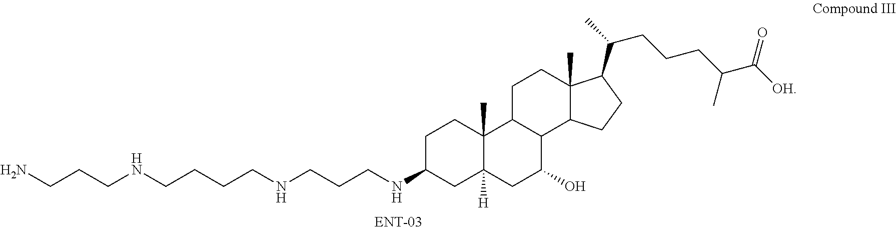 [SMILES: CC(CCC[C@@H](C)[C@H]1CCC2C3C(CC[C@@]21C)[C@@]1(C)CC[C@H](NCCCNCCCCNCCCN)C[C@@H]1C[C@H]3O)C(=O)O]

As described in PCT/US2020/044390, it was discovered that Compound III is found in subdural hematoma fluid in humans and in mouse pup brains. Compound III-R (Compound III having a C₂₅ stereocenter of configuration R may synthesized in the brain through the condensation of spermine and Compound I, as shown below.

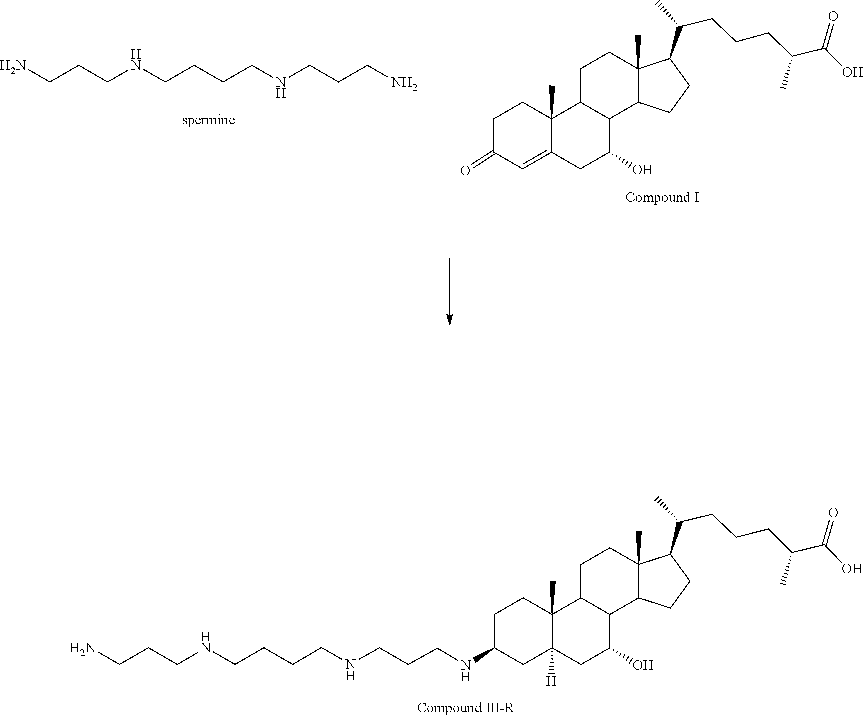 [SMILES: NCCCNCCCCNCCCN ; C[C@H](CCC[C@@H](C)[C@H]1CCC2C3C(CC[C@@]21C)[C@@]1(C)CCC(=O)C=C1C[C@H]3O)C(=O)O ; C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)O]

Compound I is believed to arise from the metabolism of 27-hydroxycholesterol (also called (25R)26-dihydroxycholesterol), a biologically active oxysterol released into the circulation from many tissues, including vascular endothelium and macrophages (Griffiths et al., 2019; Bjorkhem et al., 2002; Javitt et al., 2002). Within the brain, 27-hydroxycholesterol undergoes successive metabolism by CYP27A1, CYP7B1, and HSD-3B7, possibly in that sequence (Meaney et al., 2007). The synthesis of Compound III-R would require three additional biosynthetic steps: the reduction of the double bond in the cholesterol A ring to create the 5 alpha hydrogen; the condensation of spermine with the 3-oxo group to form the imine; and the subsequent reduction of the imine. The first step is likely catalyzed by a brain steroid 5 alpha-reductase (Celotti et al., 1992). The missing biosynthetic link is the enzyme that couples the spermine to the bile acid.

### A. Summary of Experimental Results

A synthetic sequence has been discovered, whereby the C₂₅ stereocenter of compounds of Formula I may be stereodefined to result in compounds having high de. The procedure involves asymmetric hydrogenation as shown below to define the C₂₅ stereocenter using an in situ generated asymmetric hydrogenation catalyst. Greater than five hundred variations of metal precursors, chiral ligands, additives, solvents, pressures, temperatures, and catalyst loadings were evaluated to identify the ones to accomplish the best in terms of speed of conversion, practicality, and stereoselectivity.

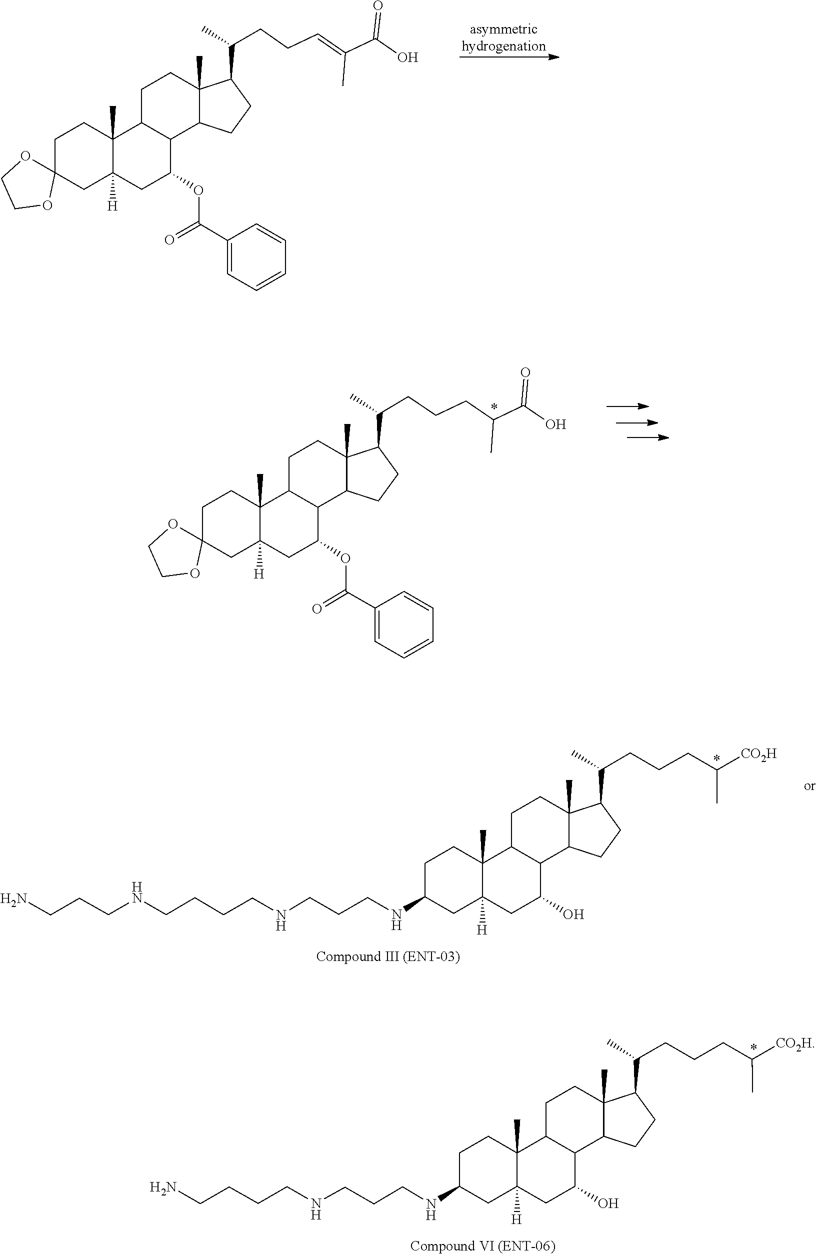 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O ; CC(CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O ; CC(CCC[C@@H](C)[C@H]1CCC2C3C(CC[C@@]21C)[C@@]1(C)CC[C@H](NCCCNCCCCNCCCN)C[C@@H]1C[C@H]3O)C(=O)O ; CC(CCC[C@@H](C)[C@H]1CCC2C3C(CC[C@@]21C)[C@@]1(C)CC[C@H](NCCCNCCCCN)C[C@@H]1C[C@H]3O)C(=O)O]

### II. Compositions

### A. Stereodefined Aminosterol Compounds

In one aspect, an aminosterol compound of Formula I-R is provided:

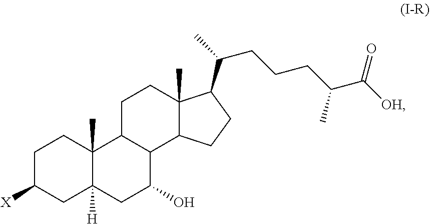

or a pharmaceutically acceptable salt, solvate, prodrug, or derivative thereof, wherein X is:

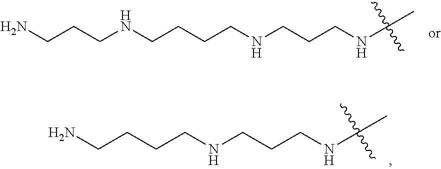 [SMILES: *NCCCNCCCCNCCCN ; *NCCCNCCCCN]

In some embodiments, the compound of Formula I-R is of formula:

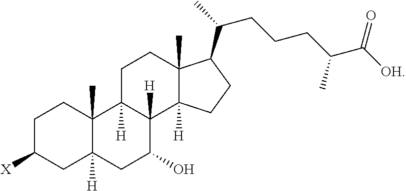

In one aspect, an aminosterol compound of Formula I-S is provided:

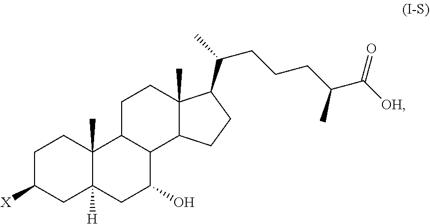

or a pharmaceutically acceptable salt, solvate, prodrug, or derivative thereof, wherein X is:

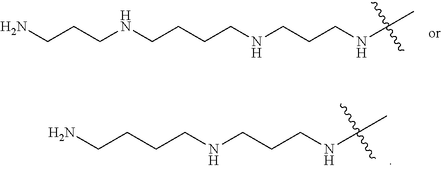 [SMILES: *NCCCNCCCCNCCCN ; *NCCCNCCCCN]

In some embodiments, the compound of Formula I-S is of formula:

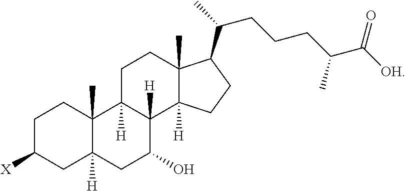

In some embodiments, the diastereomeric excess (de), with respect to the C₂₅ carbon is about 80% to about 90%, about 90% to about 95%, about 95% to about 99%, about 99% to about 99.9%, or about 100%.

The de may be assessed by any method known to those of skill in the art, for example, Mosher's ester analysis or ester formation of the acid using Pirkle's alcohol (Dale et al., (1969). “α-methoxy-α-trifluoromethylphenylacetic acid, a versatile reagent for the determination of enantiomeric composition of alcohols and amines”. Journal of Organic Chemistry. 34 (9): 2543-2549 and Pirkle et al., (1977). “Nuclear magnetic resonance determination of enantiomeric composition and absolute configuration of 7-lactones using chiral 2,2,2-trifluoro-1-(9-anthryl)ethanol”. J. Org. Chem. 42 (2): 384-387). The de may also be determined using nuclear magnetic resonance analysis (NMR). Other NMR techniques which may be used to assess de, include, but are not limited to NMR utilizing nuclear Overhauser effect (NOE), NOESY, ROESY, COSY, TOCSY, 2D-INADEQUATE, 2D-ADEQUATE, HSQC, HMQC and HMBC. Non NMR techniques include liquid chromatography mass spectrometry (LCMS), which can separate diastereomers. Triple quadrupole techniques may be used to assess distinctions in fragmentation between different isomers. Chiral or non-chiral stationary phases may be used to affect separation of isomers for the assessment of de using LCMS.

In some embodiments, the compound is:

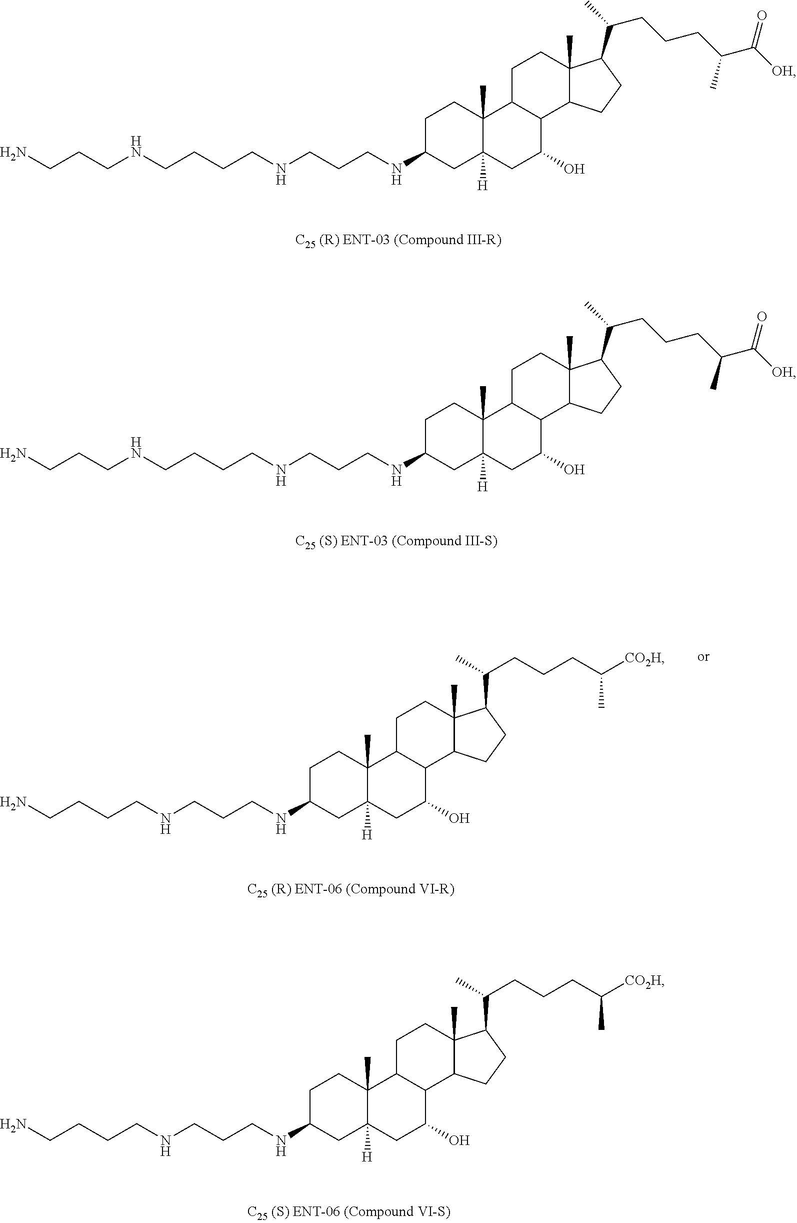 [SMILES: C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)O ; C[C@H](CCC[C@H](C)C(=O)O)[C@H]1CCC2C3C(CC[C@@]21C)[C@@]1(C)CC[C@H](NCCCNCCCCNCCCN)C[C@@H]1C[C@H]3O ; C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O ; "or" ; C[C@H](CCC[C@H](C)C(=O)O)[C@H]1CCC2C3C(CC[C@@]21C)[C@@]1(C)CC[C@H](NCCCNCCCCN)C[C@@H]1C[C@H]3O]

or a pharmaceutically acceptable salt, solvate, prodrug, or derivative thereof

### B. C₂₅ Stereodefined Synthetic Intermediates

The disclosure is also directed to synthetic intermediates with the corresponding C₂₅ stereodefinition which may be used to synthesize the aminosterols described above. Said intermediates have the formula of Compound I. Thus, in one aspect, a compound having the formula of Compound I-R is provided:

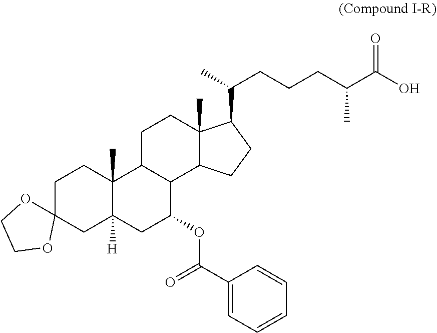 [SMILES: C[C@H](CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

or a pharmaceutically acceptable salt, solvate, or derivative thereof

In some embodiments, the compound is of formula:

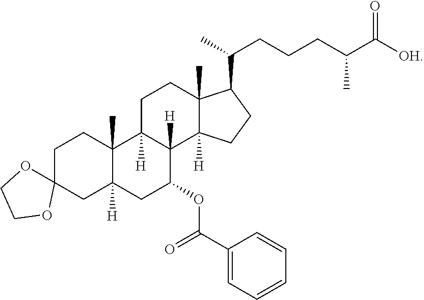

In one aspect, a compound having the formula of Compound I-S is provided:

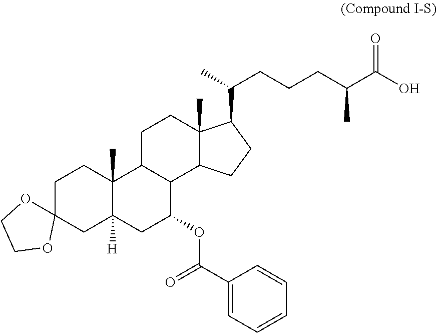 [SMILES: C[C@H](CCC[C@H](C)C(=O)O)[C@H]1CCC2C3C(CC[C@@]21C)[C@@]1(C)CCC2(C[C@@H]1C[C@H]3OC(=O)c1ccccc1)OCCO2]

or a pharmaceutically acceptable salt, solvate, or derivative thereof.

In some embodiments, the compound is of formula:

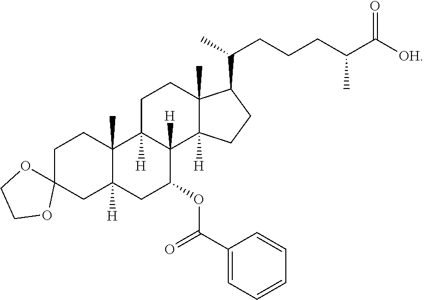

In some embodiments, the diastereomeric excess (de), with respect to the C₂₅ carbon is about 80% to about 90%, about 90% to about 95%, about 95% to about 99%, about 99% to about 99.9%, or about 100%. The de may be assessed using any of the techniques described above to assess de of the aminosterol compounds.

### C. Pharmaceutical Carriers

In another aspect, provided herein are compositions comprising an aminosterol compound disclosed herein, or a pharmaceutically acceptable salt, solvate, prodrug, or derivative thereof, and one or more pharmaceutically acceptable carriers and/or excipients. Administration of an aminosterol disclosed herein, or a pharmaceutically acceptable salt, solvate, or prodrug thereof may comprise administration of the composition.

While it is possible for an aminosterol, or a pharmaceutically acceptable salt, solvate or prodrug thereof, to be administered alone, it is preferable to administer it as a pharmaceutical formulation, together with one or more pharmaceutically acceptable carriers. The carrier(s) must be “acceptable” in the sense of being compatible with the aminosterol, or a pharmaceutically acceptable salt, solvate or prodrug thereof, and not deleterious to the recipients thereof.

Generally, the formulations are prepared by contacting an aminosterol described herein, or a pharmaceutically acceptable salt, solvate, prodrug, or derivative thereof, uniformly and intimately with liquid carriers or finely divided solid carriers or both. Then, if necessary, the product is shaped into the desired formulation. Non-aqueous vehicles such as fixed oils and ethyl oleate are also useful herein, as well as liposomes.

The carrier suitably comprises minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as gelatin, serum albumin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

In instances where aerosol administration is appropriate, an aminosterol described herein, or a pharmaceutically acceptable salt, solvate or prodrug thereof, can be formulated as an aerosol using standard procedures. The term “aerosol” includes any gas-borne suspended phase of a compound described herein which is capable of being inhaled into the bronchioles or nasal passages, and includes dry powder and aqueous aerosol, and pulmonary and nasal aerosols. Specifically, aerosol includes a gas-born suspension of droplets of a compound described herein, as may be produced in a metered dose inhaler or nebulizer, or in a mist sprayer. Aerosol also includes a dry powder composition of a composition of the present technology suspended in air or other carrier gas, which may be delivered by insufflation from an inhaler device, for example. See Ganderton & Jones, Drug Delivery to the Respiratory Tract (Ellis Horwood, 1987); Gonda, Critical Reviews in therapeutic Drug Carrier Systems, 6:273-313 (1990); and Raeburn et al., Pharmacol. Toxicol. Methods, 27:143-159 (1992).

### D. Dosage Forms

The aminosterol compositions may conveniently be presented in unit dosage form and may be prepared by any of the methods well known in the art of pharmacy. Exemplary aminosterol dosage forms include, but are not limited to, oral, intranasal, and injectable (IP, IV, or IM). Preferably, the aminosterol formulation is administered orally, intranasally, or a combination thereof. In yet another embodiment, administration comprises non-oral administration.

Formulations or compositions of the present technology may be packaged together with, or included in a kit with, instructions or a package insert. “Pharmaceutically acceptable carrier” refers to a non-toxic solid, semisolid or liquid filler, diluent, encapsulating material or formulation auxiliary of any type.

Pharmaceutical compositions according to the present technology may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™)

Suitable lubricants, including agents that act on the flowability of the powder to be compressed, may include colloidal silicon dioxide, such as Aerosil® 200, talc, stearic acid, magnesium stearate, calcium stearate, and silica gel.

Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like.

Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress*; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

### E. Dosages & Dosing Period

Dosage of an aminosterol described herein can range from about 1 to about 500 mg/day, or any amount in-between these two values. In some embodiments, a subject is administered a therapeutically effective dose of an aminosterol described herein. The therapeutically effect amount of the at least one aminosterol or a salt or derivative thereof in the methods of the disclosure can be, for example, about 0.1 to about 20 mg/kg, about 0.1 to about 15 mg/kg, about 0.1 to about 10 mg/kg, about 0.1 to about 5 mg/kg, or about 0.1 to about 2.5 mg/kg body weight of the subject. In another aspect, the therapeutically effect amount of the at least one aminosterol or a salt or derivative thereof in the methods of the disclosure can be, for example, about 0.001 to about 500 mg/day, about 0.001 to about 250 mg/day, about 0.001 to about 125 mg/day, about 0.001 to about 50 mg/day, about 0.001 to about 25 mg/day, or about 0.001 to about 10 mg/day.

Oral dosage of an aminosterol described herein can range from about 1 to about 500 mg/day, or any amount in-between these two values. In one embodiment, the method of administration comprises oral administration and the therapeutically effective amount of the aminosterol comprises (i) about 1 to about 300 mg/day; (ii) about 25 to about 300 mg/day; (iii) about 50 to about 300 mg/day; or (iv) about 75 to about 300 mg/day. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an aminosterol are much lower than oral dosages of the aminosterol. Examples of such intranasal aminosterol low dosages include, but are not limited to, about 0.001 to about 6 mg/day, or any amount in-between these two values. In some embodiments, the method of administration comprises nasal administration and the therapeutically effective amount of the aminosterol comprises (i) about 0.001 to about 6 mg/day; (ii) about 0.001 to about 4 mg/day; or (iii) about 0.001 to about 2 mg/day. For example, the low dosage of an intranasally administered aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that the same dosage would not provide any pharmacological effect if administered by any other route—e.g., a “subtherapeutic” dosage, and, in addition, does not result in negative effects. For example, as described herein, Compound III (ENT-03) has the pharmacological effects of a reduction in food intake and weight loss. Therefore, in certain embodiments of the IN methods of the disclosure, if the aminosterol may be Compound III (ENT-03) or a salt, solvate, prodrug, or derivative thereof, then if the same IN Compound III dosage is administered via another route, such as oral, IP, or IV, then the Compound III dosage will not result in a noticeable reduction in food intake or noticeable weight loss. Similarly, some aminosterols are known to produce the pharmacological effects of nausea, vomiting and/or reduced blood pressure. Thus, in certain embodiments of the IN methods of the disclosure, if the aminosterol has this effect when given IN, then if the same IN aminosterol dosage is administered via another route, such as oral, IP, or IV, then the aminosterol dosage will not result in noticeable nausea, vomiting, and/or a reduction in blood pressure. In some embodiments, intranasal administration comprises delivery of the aminosterol to the brain. Suitable exemplary aminosterol dosages are described above.

Aminosterol doses can be de-escalated (reduced) if any given aminosterol dose induces a persistent undesirable side effect, such as diarrhea, vomiting, or nausea. In another embodiment, a dose of an aminosterol can be varied plus or minus a defined amount to enable a modest reduction in a dose to eliminate adverse events, or a modest increase in a dose if clinical results suggest this is desirable—e.g., no or minimal adverse events and potential increased efficacy with a modest increase in dose. For example, in one embodiment an aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

The pharmaceutical composition comprising an aminosterol or a derivative, salt, solvate, or prodrug thereof can be administered for any suitable period of time, including as a maintenance dose for a prolonged period of time. Dosing can be done on an as needed basis using any pharmaceutically acceptable dosing regimen. Aminosterol dosing can be no more than lx per day, once every other day, once every three days, once every four days, once every five days, once every six days, once a week, or divided over multiple time periods during a given day (e.g., twice daily). In an exemplary embodiment, dosing is 1×/day.

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 min, about 60 min, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the aminosterol dose in the morning enables the synchronization of all the autonomic physiological functions occurring during the day. In other embodiments of the disclosure, the aminosterol dosage is taken within about 15 min, about 30 min, about 45 min, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the disclosure, following the aminosterol dosage the subject has a period of about 15 min, about 30 min, about 45 min, about 1 hours, about 1.25 hrs, about 1.5 hrs, about 1.75 hrs, about 2 hrs, about 2.25 hrs, about 2.5 hrs, about 2.75 hrs, or about 3 hours without food.

### F. “Fixed Aminosterol Dose”

In one aspect, the present application relates to the discovery of a method to determine a “fixed dose” of an aminosterol described herein, that is not age, size, or weight dependent but rather is individually calibrated. The “fixed dose” obtained through this method yields highly effective results in treating the symptom(s) based on which the “fixed dose” was determined, related symptoms along the “brain-gut” axis, and the underlying disorder. Further, contemplated herein are methods of leveraging this same “fixed dose” method for methods of prevention of the underlying disorder. The present disclosure is not limited to methods whereby a fixed aminosterol dosage is determined for a specific patient.

A “fixed aminosterol dose,” also referred to herein as a “fixed escalated aminosterol dose,” which will be therapeutically effective is determined for each patient by establishing a starting dose of an aminosterol composition and a threshold for improvement of a particular symptom which is used as a tool or marker for evaluating the effectiveness of the aminosterol dosage. Following determining a starting aminosterol dosage for a particular patient, the aminosterol dose is then progressively escalated by a consistent amount over consistent time intervals until the desired improvement is achieved; this aminosterol dosage is the “fixed escalated aminosterol dosage” for that particular patient for that particular symptom. In exemplary embodiments, an orally administered aminosterol dose is escalated every about 3 to about 5 days by about 25 mg until the desired improvement is reached. Symptoms evaluated, along with tools for measuring symptom improvement, may be specifically described below, including but not limited to constipation, hallucinations, sleep disturbances (e.g. REM disturbed sleep or circadian rhythm dysfunction), cognitive impairment, depression, or alpha-synuclein aggregation.

This therapeutically effective “fixed dose” is then maintained throughout treatment and/or prevention. Thus, even if the patient goes “off drug” and ceases taking the aminosterol composition, the same “fixed dose” is taken with no ramp up period following re-initiation of aminosterol treatment. Not to be bound by theory, it is believed that the aminosterol dose is dependent on the severity of nerve damage relating to the symptom establishing the “fixed dose” threshold—e.g. for constipation, the dose may be related to the extent of nervous system damage in the patient's gut.

Dose escalation: When determining a “fixed aminosterol dosage” for a particular patient, a patient is started at a lower dose and then the dose is escalated until a positive result is observed for the symptom being evaluated. An exemplary symptom to be evaluated can be constipation, but any symptom associated with the disease or disorder to be treated can be used as a marker for evaluating aminosterol dosage. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a patient experiencing severe constipation, defined as less than one spontaneous bowel movement (SBM) a week, the starting oral aminosterol dose can be about 150 mg/day or greater. In contrast, for a patient having moderate constipation, e.g., defined as having more than one SBM a week, the starting oral aminosterol dose can be about 75 mg/day. Thus, as an example, a patient experiencing moderate constipation can be started at an oral aminosterol dosage of about 75 mg/day, whereas a patient experiencing severe constipation can be started at an oral aminosterol dosage of about 150 mg/day.

In other embodiments, a patient experiencing moderate symptoms (for the symptom being used to calculate a fixed escalated aminosterol dose) can be started at an oral aminosterol dosage of from about 10 mg/day to about 75 mg/day, or any amount in-between these values. For example, the starting oral aminosterol dosage for a moderate symptom can be about 10, about 15, about 20, about 25, about 30, about 35, about 40, about 45, about 60, about 65, about 70, or about 75 mg/day.

In yet further embodiments, when the patient is experiencing severe symptoms (for the symptom being used to calculate the fixed escalated aminosterol dose), the patient can be started at an oral aminosterol dosage ranging from about 75 to about 175 mg/day, or any amount in-between these two values. For example, the starting oral aminosterol dosage for a severe symptom can be about 75, about 80, about 85, about 90, about 95, about 100, about 105, about 110, about 115, about 120, about 125, about 130, about 135, about 140, about 145, about 150 about 155, about 160, about 165, about 170, or about 175 mg/day.

In some embodiments, the starting oral aminosterol dose may be about 125 mg or about 175 mg/day; again dependent on the severity of the symptom, such as constipation.

Starting intranasal (IN) aminosterol dosages prior to dose escalation can be, for example, about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In exemplary embodiments, the aminosterol dose is given periodically as needed. For example, the aminosterol dose can be given once per day. The aminosterol dose can also be given every other day, 2, 3, 4, or 5× per week, once/week, or 2×/week. In another embodiment, the aminosterol dose can be given every other week, or it can be given for a few weeks, followed by skipping a few weeks (as the effects persist following treatment), followed by restarting aminosterol treatment.

When calculating a fixed escalated aminosterol dose, the dose can be escalated following any suitable time period. In one embodiment, the aminosterol dose is escalated every about 3 to about 7 days by about a defined amount until a desired improvement is reached. For example, when the symptom being treated/measured is constipation, threshold improvement can be an increase of one SBM per week or at least a total of three bowel movements per week. In other embodiments, the aminosterol dose can be escalated every about 1, about 2, about 3, about 4, about 5, about 6, about 7, about 8, about 9, about 10, about 11, about 12, about 13, or about 14 days. In other embodiments, the aminosterol dose can be escalated about 1×/week, about 2×/week, about every other week, or about lx/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Other symptoms that can be used as an endpoint to determine aminosterol dosage for a patient's fixed escalated aminosterol dosage are any symptom known to be associated with the disease, disorder, or condition intended to be treated. For example, neurodisease symptoms described herein and include, but are not limited to, (a) at least one non-motor aspect of experiences of daily living as defined by Part I of the Unified Parkinson's Disease Rating Scale (UPDRS), such as for example cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the UPDRS, such as for example, speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the UPDRS, such as for example, speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the UPDRS, such as for example, dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (1) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature; (t) improvement in breathing or apnea; (u) correction of cardiac conduction defect; (v) amelioration of pain; (w) restoration of bladder sensation and urination; (x) urinary incontinence; and/or (y) control of nocturia.

### III. Methods of Synthesis of C₂₅ Stereodefined Synthetic Intermediates and Aminosterols

As described above in Section I.A, the present disclosure is directed to methods of synthesis of C₂₅ stereodefined synthetic intermediates and aminosterols. The methods employ a key asymmetric hydrogenation of Compound II to produce Compound I having the desired stereochemical configuration, R or S, at the C₂₅ carbon.

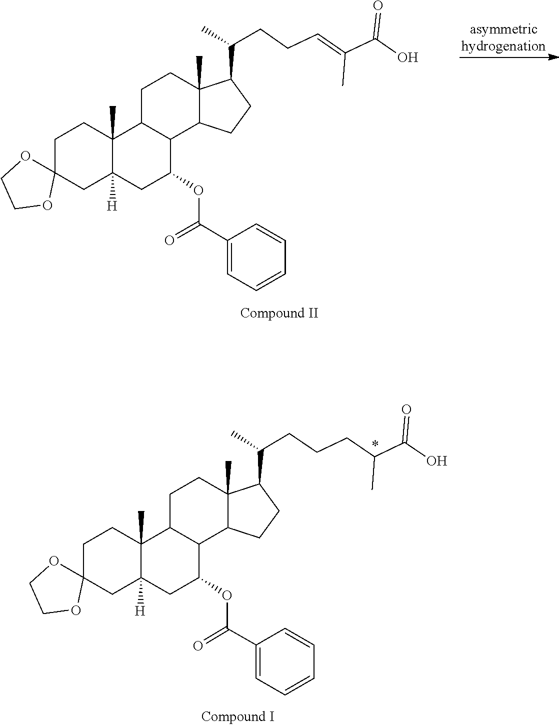 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O ; CC(CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

In methods wherein the R configuration at C₂₅ is desired asymmetric hydrogenation may comprise mixing Compound IV:

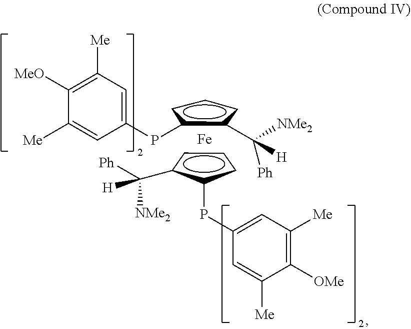

(Me-Allyl)2Ru(COD), and an acid to form a solution, and contacting Compound II with the solution and hydrogen (112).

In methods wherein the S configuration at C₂₅ is desired asymmetric hydrogenation may comprise mixing Compound V:

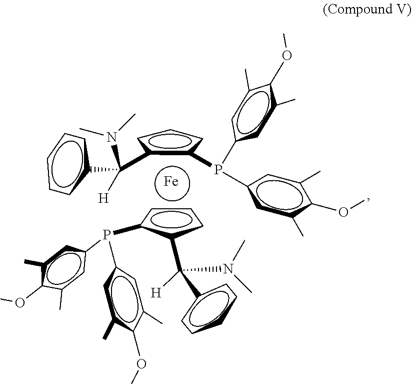

(Me-Allyl)2Ru(COD), and an acid to form a solution, and contacting Compound II with the solution and hydrogen (112).

In some embodiments, the acid comprises HBr, HCl, or HI. In some embodiments, the solution further comprises isopropyl acetate.

In some embodiments, the asymmetric hydrogenation comprises subjecting Compound II to about 60 psi to about 70 psi, about 70 psi to about 80 psi, about 80 psi to about 90 psi, about 90 psi to about 100 psi, about 100 psi to about 120 psi, about 120 psi to about 140 psi, about 140 psi to about 160 psi, about 160 psi to about 180 psi, about 180 psi to about 200 psi, about 200 psi to about 250 psi, about 250 psi to about 300 psi, about 300 psi to about 350 psi, about 350 psi to about 400 psi, about 400 psi to about 500 psi, about 500 psi to about 550 psi, about 550 psi to about 600 psi, about 600 psi to about 650 psi, or about greater than about 650 psi, of hydrogen (H₂).

In some embodiments, the asymmetric hydrogenation is at a temperature of about 20° C. to about 30° C., about 30° C. to about 40° C., about 40° C. to about 50° C., about 50° C. to about 60° C., or about greater than 60° C.

In some embodiments, the asymmetric hydrogenation is for about 12 hr to about 24 hr, about 24 hr to about 36 hr, about 36 hr to about 48 hr, about 48 hr to about 60 hr, or greater than about 60 hr.

In some embodiments: (a) the mol % s of Compound IV or Compound V, and (Me-Allyl)2Ru(COD) are about 0.2% to about 1%, about 1% to about 3%, about 3% to about 4%, about 4% to about 5%, about 5% to about 6%, or about 6% to about 8%, relative to Compound II; and (b) the mol % of the acid is about 4% to about 6%, about 6% to about 8%, about 8% to about 10%, about 10% to about 12%, or about 12% to about 16%, relative to Compound II.

In some embodiments, the diastereomeric excess (de) of the C₂₅ carbon is about 80% to about 90%, about 90% to about 95%, about 95% to about 99%, about 99% to about 99.9%, or about 100%.

Alternatively, conditions employing alternative reagents and catalysis may be substituted to affect the asymmetric hydrogenation. Such conditions and catalysis will be known to those having ordinary skill in the art, for example, those discussed in Ratovelomanana-Vidal et al., “Asymmetric Hydrogenation and Transfer Hydrogenation,” Edition April 2021 Wiley-VCH, Weinheim; or in Ayad, et al., “Transition-Metal-Catalyzed Asymmetric Hydrogenation and Transfer Hydrogenation: Sustainable Chemistry to Access Bioactive Molecules,” 2016 16:(6), pp. 2754-2771; the entire disclosures of which are hereby incorporated by reference in their entireties.

After hydrogenation, Compound I (i.e., Compound I-S or Compound I-R) may be further reacted to furnish the aminosterol compounds described above. These methods may further comprise hydrolysis of the dioxolane of Compound I to form a ketone and reductive amination of the ketone with amine (spermine or spermidine):

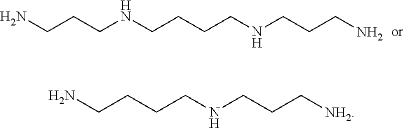 [SMILES: NCCCNCCCCNCCCN ; NCCCCNCCCN]

The synthesis of the aminosterol compounds of the disclosure (Compounds of Formula I-R or Formula I-S) may be synthesized from Compound I as shown below (* denotes enrichment of R or S configuration at C₂₅).

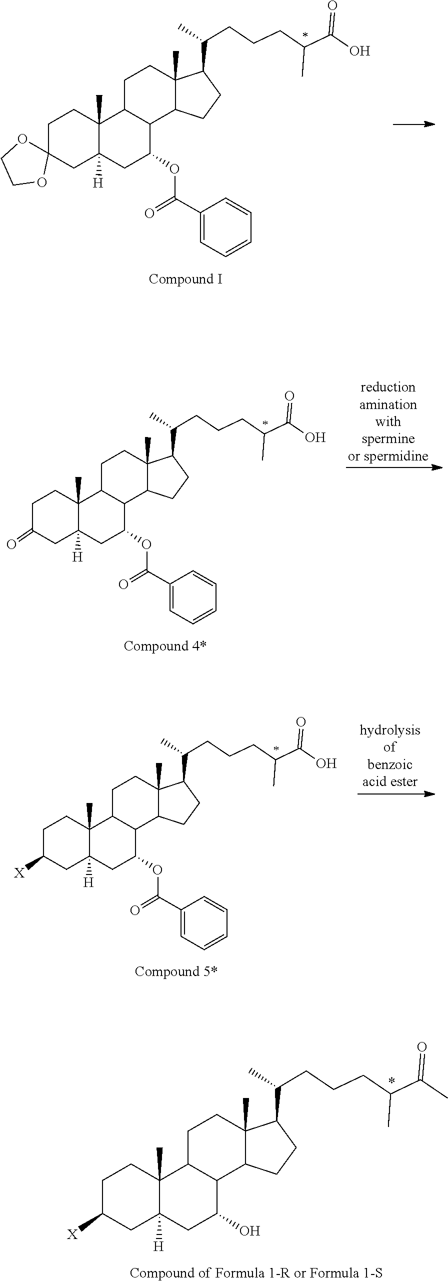

Hydrolysis of the dioxolane (ketal) of Compound I may be affected by subjection to acidic conditions to form the ketone Compound 4* (see Compound 4 of the Examples) having the C₂₅ carbon in high de. A variety of acids may be used, not limited to, HCl or toluenesulfonic acid. Said ketone may then be subjected to reductive amination with spermine or spermidine to produce Compound 5* followed by hydrolysis under basic conditions to release benzoic acid to furnish the aminosterols of the disclosure. Bases not limited to KOH or NaOH may be used for said hydrolysis of Compound 5*. For the reductive amination, the ketone Compound 4* may be mixed with spermine or spermidine to form an imine or iminium intermediate in situ which is subsequently treated with a reducing reagent such as NaBH₄, LiAlH₄, or NaBH₄·CeCl₃. Methods and conditions to affect reductive amination are known to those of ordinary skill in the art. See, for example, Dangerfield et al., J. Org. Chem., 2010, 75, 5470-5477; Taibakhsh et al., Synthesis, 2011, 490-496; and Abdel-Magid et al., J. Org. Chem., 1996, 61, 3849-3862; the entire disclosures of which are hereby incorporated by reference.

### IV. Methods of Treatment and/or Prevention

Aspects of this disclosure relate to methods of treating certain symptoms and/or methods of treating and/or preventing diseases or disorders associated with one or more of these symptoms by administration of a therapeutically effective amount of an aminosterol disclosed herein (e.g., ENT-03 (Compound III-R or -S) or ENT-06 (Compound VI-R or -S) or another aminosterol described herein), or a pharmaceutically acceptable salt, solvate, prodrug, or derivative thereof, optionally present in one or more pharmaceutically acceptable carriers. The therapeutically effective amount can be as described herein, which includes but is not limited to a “fixed aminosterol dosage” determined as described herein.

In one embodiment, the symptoms, diseases, and/or disorders are generally correlated with abnormal αS pathology and/or dopaminergic dysfunction, which means they are amenable to treatment with aminosterols described herein. The compositions of the present technology can be administered using any pharmaceutically acceptable method, including but not limited to oral, pulmonary, nasal, and nebularization administration. In yet another embodiment, administration comprises non-oral administration.

In some embodiments, provided herein are methods for treating a subject in need having a condition or symptom susceptible to treatment with an aminosterol, comprising administering to the subject a therapeutically effective amount of an aminosterol described herein, or a pharmaceutically acceptable salt, solvate, or prodrug thereof. In some embodiments, provided herein are methods for treating a subject in need having a condition susceptible to treatment with an aminosterol, comprising administering to the subject a therapeutically effective amount of a composition comprising or consisting essentially of an aminosterol disclosed herein, or a pharmaceutically acceptable salt, solvate, or prodrug thereof, and one or more pharmaceutically acceptable carriers and/or excipients.

Non-limiting examples of symptoms amenable to treatment with aminosterols include but are not limited to constipation, hallucinations, sleep disorders, cognitive impairment, depression, and inflammation.

Examples of diseases amenable to treatment with aminosterols are described herein and include but are not limited to those described herein, such as neurological diseases, e.g., PD, AD, MSA, schizophrenia, Huntington's disease (HD), Progressive supranuclear palsy, Frontotemporal dementia (FTD), vascular dementia, Amyotrophic lateral sclerosis (ALS), multiple sclerosis (MS), spinal muscular atrophy (SMA), Friedreich's ataxia. In another embodiment, the aminosterols described herein and compositions comprising the same can be used in methods of treating, preventing, and/or slowing the onset or progression of psychological or behavior disorder and/or a related symptom in a subject in need is provided, In one embodiment, the psychological or behavior disorder can be, for example, depression, anxiety, delirium, irritability, illusion and delusions, amnesia, autism, apathy, bipolar disorder, disinhibition, aberrant motor and obsessive-compulsive behaviors, sleep disorders, sleep fragmentation, REM behavior disorder, circadian rhythm dysfunction, sleep apnea, and cognitive impairment. In another embodiment, a method of treating, preventing, and/or slowing the onset or progression of a cerebral or general ischemic disorder and/or a related symptom in a subject in need is provided. The cerebral or general ischemic disorder can be, for example, microangiopathy, intrapartum cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, and pulmonary edema.

In one embodiment, a method of inhibiting protein tyrosine phosphatase 1B (PTP1B) is provided, comprising contacting PTP1B with at least one aminosterol disclosed herein, or pharmaceutically acceptable salt, solvate, or prodrug thereof.

Applicant has shown in Example 4, that squalamine can increase transcription in the gut of old mice, thus having a rejuvenating effect on the gut. It is believed that this activity extends to ENT-03 (Compound III) and derivatives thereof. Thus, in another aspect, a method of increasing transcription in the gut of a subject is provided, the method comprising administering to the subject a therapeutically effective amount of an aminosterol compound of any embodiment herein, or a pharmaceutically acceptable salt, solvate, prodrug, or derivative thereof.

Exemplary symptoms correlated with abnormal αS pathology and/or dopaminergic dysfunction and amenable to aminosterol treatment include, for example, constipation, hallucinations, and other symptoms described herein.

A. Inflammation Related to Abnormal αS Pathology and/or Dopaminergic Dysfunction and Amenable to Aminosterol Treatment

In one embodiment, provided is a method of treating, preventing, and/or slowing the onset or progression in a subject of inflammation and/or a related symptom related to αS pathology. The method comprises administering to the subject a therapeutically effective amount of at least one aminosterol disclosed herein (e.g., ENT-03 (Compound III-R or -S) or ENT-06 (Compound VI-R or -S)), or pharmaceutically acceptable salt, solvate, or prodrug thereof.

αS is a potent pro-inflammatory hormone. Inflammation can be blocked by either of two strategies. First, inflammation can be blocked by reducing the tissue concentration of αS by decreasing or stopping production of αS. Alternatively, inflammation can be blocked by interrupting the signaling between αS and inflammatory cells that express CD1lb. The subject of the methods of the disclosure can be any mammal, including a human.

The inflammatory disease or condition caused by excessive expression of neuronal αS can be a neurodegenerative disorder (NDD), such as an alpha-synucleinopathy. Exemplary alpha-synucleinopathies include, but are not limited to, PD, Lewy body dementia, multiple system atrophy, amyotrophic lateral sclerosis, Huntington's chorea, multiple sclerosis or schizophrenia. In other embodiments, the inflammatory disease or condition caused by excessive expression of neuronal alpha synuclein can be an autoimmune disease, a chronic inflammatory disease, or an autoinflammatory disease. In other embodiments, the inflammatory disease or condition caused by excessive expression of neuronal αS can be selected from the group consisting of asthma, chronic peptic ulcer, tuberculosis, chronic periodontitis, chronic sinusitis, chronic active hepatitis, psoriatic arthritis, gouty arthritis, acne vulgaris, osteoarthritis, rheumatoid arthritis, lupus, systemic lupus erythematosus, multiple sclerosis, ankylosing spondylitis, Crohn's disease, psoriasis, primary sclerosing cholangitis, ulcerative colitis, allergies, inflammatory bowel diseases, Celiac disease, Chronic prostatitis, diverticulitis, dermatomyositis, polymyositis, systemic sclerosis, glomerulonephritis, hidradenitis suppurativa, hypersensitivities, interstitial cystitis, otitis, pelvic inflammatory disease, reperfusion injury, rheumatic fever, sarcoidosis, transplant rejection, and vasculitis.

### B. Diseases or Disorders Amenable to Aminosterol Treatment

The aminosterols described herein (e.g., ENT-03 (Compound III-R or -S) or ENT-06 (Compound VI-R or -S)), including a pharmaceutically acceptable salt, solvate, prodrug, or derivative thereof, can be used in methods of treating and/or preventing a variety of diseases and disorders, which are generally correlated with abnormal αS pathology and/or dopaminergic dysfunction, as described herein and as described below.

In one embodiment, provided is a method of treating, preventing, and/or slowing the onset or progression in a subject of diseases or disorder correlated with abnormal αS pathology and/or dopaminergic dysfunction and/or a related symptom related to αS pathology. The method comprises administering to the subject a therapeutically effective amount of at least one aminosterol disclosed herein (e.g., ENT-03 (Compound III-R or -S) or ENT-06 (Compound VI-R or -S)), or pharmaceutically acceptable salt, solvate, prodrug, or derivative thereof.

Examples of exemplary disease and disorders that can be treated and/or prevented using the aminosterols described herein include, but are not limited to, neurological or neurodegenerative disorders or diseases. Examples of neurological or neurodegenerative disorders or diseases include, but are not limited to, Alzheimer's disease (AD), Parkinson's disease (PD), Huntington's Disease, Multiple Sclerosis, Amyotrophic Lateral Sclerosis (ALS), multiple system atrophy (MSA), spinal muscular atrophy (SMA), schizophrenia, Friedreich's ataxia, vascular dementia, Lewy Body dementia or disease, spinal muscular atrophy, supranuclear palsy, frontotemporal dementia, progressive nuclear palsy, progressive supranuclear palsy, Guadeloupian parkinsonism, spinocerebellar ataxia, and autism.

In another embodiment, a neurodegenerative disorder may be positively impacted by administration of a therapeutically effective amount of an aminosterol according to the disclosure. A “positive impact” includes for example slowing advancement of the condition, improving one or more symptoms, etc.

Also encompassed are methods of treating and/or preventing psychological or behavior disorder, or any symptom related to such diseases or conditions. In one embodiment, the psychological or behavior disorder is depression, anxiety, delirium, irritability, illusion and delusions, amnesia, autism, apathy, bipolar disorder, disinhibition, aberrant motor and obsessive-compulsive behaviors, sleep disorders, sleep fragmentation, REM behavior disorder, circadian rhythm dysfunction, sleep apnea, or cognitive impairment

In another embodiment, psychological or behavior disorder, or any symptom related to such diseases or conditions, may be positively impacted by administration of a therapeutically effective amount of an aminosterol according to the disclosure. A “positive impact” includes for example slowing advancement of the condition, improving one or more symptoms, etc.

In one embodiment, a method of treating, preventing, and/or slowing the onset or progression of a cerebral or general ischemic disorder and/or a related symptom in a subject in need is provided, comprising administering to the subject a therapeutically effective amount of at least one aminosterol disclosed herein (e.g., ENT-03 (Compound III-R or -S) or ENT-06 (Compound VI-R or -S)), or pharmaceutically acceptable salt, solvate, prodrug, or derivative thereof. Examples of cerebral or general ischemic disorders include but are not limited to erectile dysfunction, blood pressure (both high and low blood pressure), and cardiac conduction defects.

In one embodiment, a method of suppressing, preventing and/or slowing the onset or progression of appetite and/or one or more related symptoms in a subject in need is provided, comprising administering to the subject a therapeutically effective amount of at least one aminosterol disclosed herein (e.g., ENT-03 (Compound III-R or -S) or ENT-06 (Compound VI-R or -S)), or pharmaceutically acceptable salt, solvate, prodrug, or derivative thereof.

In one embodiment, a method of reducing weight in a subject in need thereof is provided, comprising administering to the subject a therapeutically effective amount of an aminosterol disclosed herein (e.g., ENT-03 (Compound III-R or -S) or ENT-06 (Compound VI-R or -S)), or pharmaceutically acceptable salt, solvate, prodrug, or derivative thereof.

In some embodiments, the subject is suffering from and/or at risk of suffering from one or more conditions selected from the group consisting of obesity, fatty liver disease, type 2 diabetes, heart disease, stroke, hypertension, gallbladder disease, gout, sleep apnea, osteoarthritis, High LDL cholesterol, low HDL cholesterol, high levels of triglycerides (dyslipidemia), endometrial cancer, breast cancer, colon cancer, kidney cancer, gallbladder cancer, and liver cancer.

In one aspect, the aminosterols described herein can be administered to a subject in need to treat a microbial infection. In some embodiments, the subject in need has a condition selected from the group consisting of viral infections, microbial infections, bacterial infections, e.g., Gram-negative and/or Gram-positive bacterial infections, Mycobacteria infections, fungal infections, and/or protozoan infections.

In some embodiments, the viral infection is caused by a virus selected from the group consisting of Yellow Fever, Cytomegalovirus, Eastern Equine Encephalitis virus, Hepatitis B virus, Hepatitis Delta virus, Dengue virus, and Human Immunodeficiency virus. In some embodiments, the condition to be treated is a viral infection caused by a virus selected from the group consisting of “African Swine Fever Viruses,” Arbovirus, Adenoviridae, Arenaviridae, Arterivirus, Astroviridae, Baculoviridae, Bimaviridae, Birnaviridae, Bunyaviridae, Caliciviridae, Caulimoviridae, Circoviridae, Coronaviridae, Cystoviridae, Dengue, EBV, HIV, Deltaviridae, Filviridae, Filoviridae, Flaviviridae, Hepadnaviridae (Hepatitis), Herpesviridae (such as, Cytomegalovirus, Herpes Simplex, Herpes Zoster), Iridoviridae, Mononegavirus (e.g., Paramyxoviridae, Morbillivirus, Rhabdoviridae), Myoviridae, Orthomyxoviridae (e.g., Influenza A, Influenza B, and parainfluenza), Papiloma virus, Papovaviridae, Paramyxoviridae, Prions, Parvoviridae, Phycodnaviridae, Picomaviridae (e.g., Rhinovirus, Poliovirus), Poxviridae (such as Smallpox or Vaccinia), Potyviridae, Reoviridae (e.g., Rotavirus), Retroviridae (HTLV-I, HTLV-II, Lentivirus), Rhabdoviridae, Tectiviridae, Togaviridae (e.g., Rubivirus), herpes, pox, papilloma, corona, influenza, hepatitis, sendai, sindbis, vaccinia viruses, west nile, hanta, viruses which cause the common cold, and any combination thereof. In some embodiments, the condition to be treated is selected from the group consisting of AIDS, viral meningitis, Dengue, EBV, hepatitis, a chronic disease suspected to be of viral origin, multiple sclerosis, Type I diabetes, Type II diabetes, atherosclerosis, cardiomyopathies, Kawaski disease, aplastic anemia, and any combination thereof.

In some embodiments, the method further comprises administering one or more antiviral drugs.

In one aspect, a method of treating or preventing an infection by a coronavirus in a subject is provided, comprising administering to the subject a therapeutically effective amount of the aminosterol compound of any embodiment herein or the composition of any embodiment herein.

In some embodiments, the coronavirus comprises a virus selected from the group consisting of an Alphacoronavirus; a Colacovirus such as Bat coronavirus CDPHE15; a Decacovirus such as Bat coronavirus HKU10 or Rhinolophus ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night heron coronavirus HKU19; a Moordecovirus such as Common moorhen coronavirus HKU21; a Gammacoronavirus; a Cegacovirus such as Beluga whale coronavirus SW1; and an Igacovirus such as Avian coronavirus.

In some embodiments, the coronavirus is encoded by a polynucleotide comprising the sequence of SARS-CoV-2, or a polynucleotide having at least 80% sequence identity to the polynucleotide comprising the sequence of SARS-CoV-2. In some embodiments, the coronavirus comprises or is characteristic of human coronavirus 229E, human coronavirus OC43, SARS-CoV, HCoV NL63, HKU1, MERS-CoV, or SARS-CoV-2. In some embodiments, the coronavirus comprises or is characteristic of SARS-CoV-2.

### V. Patient Populations

The disclosed aminosterols and compositions comprising the same can be used to treat a range of subjects, including human and non-human animals, including mammals, as well as immature and mature animals, including human children and adults. The human subject to be treated can be an infant, toddler, school-aged child, teenager, young adult, adult, or elderly patient.

In embodiments disclosed herein relating to prevention, particular patient populations may be selected based on being “at risk for” the development of any of the conditions disclosed herein. For example, genetic markers of the condition or family history may be used as signs to identify subjects likely to develop the particular condition. Thus, in some embodiments, prevention may involve first identifying a patient population at risk of developing the condition. Alternatively, certain symptoms are considered early signs of particular disorders. Thus, in some embodiments, a patient population may be selected for being “at risk” for developing the condition based on age and experiencing symptoms associated with the condition. Further genetic or hereditary signs may be used to refine the patient population.

### VI. Kits

Aminosterol formulations or compositions of the disclosure may be packaged together with or included in a kit along with instructions or a package insert. Such instructions or package inserts may address recommended storage conditions, such as time, temperature and light, taking into account the shelf-life of the aminosterol or derivatives or salts thereof Such instructions or package inserts may also address the particular advantages of the aminosterol or derivatives or salts thereof, such as the ease of storage for formulations that may require use in the field, outside of controlled hospital, clinic or office conditions.

The disclosure also provides a pharmaceutical pack or kit comprising one or more containers filled with one or more aminosterol pharmaceutical compositions disclosed herein. The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat-sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

### VII. Combination Therapy

In the methods of the disclosure, the aminosterol compositions may be administered alone or in combination with one or more other therapeutic agents. An example of an additional therapeutic agent is one known to treat the condition the aminosterol is being administered to treat.

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents administered first, followed by the second. The regimen selected can be administered concurrently since activation of the aminosterol induced response does not require the systemic absorption of the aminosterol into the bloodstream and thus eliminates concern over the likelihood systemic of drug-drug interactions between the aminosterol and the administered drug.

### VIII. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an”, and “the” are used interchangeably and intended to include the plural forms as well and fall within each meaning, unless the context clearly indicates otherwise. Also, as used herein, “and/or” refers to, and encompasses, any and all possible combinations of one or more of the listed items, as well as the lack of combinations when interpreted in the alternative (“or”).

As used herein, the phrase “therapeutically effective amount” means a dose of aminosterol, or a salt or derivative thereof that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with standard practices as needed to treat a specific subject. The therapeutically effective amount may vary based on the route of administration and dosage form, the age and weight of the subject, and/or the severity of the subject's condition. For example one of skill in the art would understand that the therapeutically effective amount for treating a small individual may be different from the therapeutically effective amount for treating a large individual.

The term “administering” as used herein includes prescribing for administration, as well as actually administering, and includes physically administering by the subject being treated or by another.

As used herein “subject” or “patient” or “individual” refers to any subject, patient, or individual and the terms are used interchangeably herein. In this regard, the terms “subject,” “patient,” and “individual” includes mammals, and, in particular humans.

“Diastereomeric excess” or de, as used herein, refers to a percentage expressing the purity of a respective diastereomer (i.e., C₂₅ R or S). It may be calculated, for example for the R isomer, using the formula (R-S)/R+S, wherein “R” and “S” in the formula are amounts of each respective isomer in a mixture which may be determined using quantitative methods such as LCMS, wherein the amounts of each respective isomer in a mixture which may be determined using quantitative methods such as LCMS or HPLC (diode array or charged aerosol detection), wherein the amounts may be related to peak area or height.

A “derivative” of Compound III may have one or more chemical modifications which do not modify, or drastically diminish, or may improve, the activity of Compound III. Such “activity” may include pharmacological targets and affinity therefore, including changes in affinities for different subtypes of a particular receptor target. A “derivative” of an aminosterol or Compound III in which modifications well known in the art of medicinal chemistry to “mimic” the original spatial and charge characteristics of a portion of the original structure can be introduced to improve the therapeutic characteristics of the aminosterol. In general, such modifications are introduced to influence metabolism, ease of administration, biodistribution, or any combination thereof. Examples of such variants or derivatives include, but are not limited to, (1) substitutions of the sulfate or carboxylic acid by a sulfonate, sulfate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system. Other derivatives include replacement of one or more hydrogens of the aminosterol with deuterium or the unsaturation of any one or more C-C single bonds of the aminosterol. The pharmaceutical composition can comprise one or more pharmaceutically acceptable carriers or excipients.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention. When an embodiment is defined by one of these terms (e.g., “comprising”), it should be understood that this disclosure also includes alternative embodiments, such as “consisting essentially of” and “consisting of” for said embodiment.

“Substantially” or “essentially” means nearly totally or completely, for instance, 95%, 96%, 97%, 98%, 99%, or greater of some given quantity.

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the disclosure.

As used herein, the term “treatment” or “treating” means any treatment of a disease or condition or associated disorder, in a patient, including:

Inhibiting or preventing the disease or condition, that is, arresting or suppressing the development of clinical symptoms, such as neurological deficits resulting from cerebral ischemia, also included within “treatment” is provision of neuroprotection; and/or relieving the disease or condition that is, causing the regression of clinical symptoms (e.g., increasing neurological performance or reducing neurological deficits).

In some embodiments, “treatment” encompasses “providing neuroprotection” to the subject. “Treatment” and “providing neuroprotection” may comprise the administration of the therapeutics agent(s) or compositions disclosed herein.

“Pharmaceutically acceptable salt” refers to salts of a compound, which salts are suitable for pharmaceutical use and are derived from a variety of organic and inorganic counter ions well known in the art. Pharmaceutically acceptable salts include, when the compound contains an acidic functionality, by way of example only, sodium, potassium, calcium, magnesium, ammonium, and tetraalkylammonium. When the molecule contains a basic functionality, salts of organic or inorganic acids, such as hydrochloride, hydrobromide, tartrate, mesylate, acetate, maleate, and oxalate. Stahl and Wermuth, eds., “Handbook of Pharmaceutically Acceptable Salts,” (2002), Verlag Helvetica Chimica Acta, Zurich, Switzerland), which is hereby incorporated by reference for its teachings related to pharmaceutically acceptable salts, discusses a variety of pharmaceutical salts, their selection, preparation, and use.

“(Me-Allyl)2Ru(COD)” as used herein, refers to (1,5-Cyclooctadiene)bis(2-methylallyl)ruthenium, bis(2-methallyl)(1,5-cyclooctadiene)ruthenium, or ruthenium(II) bis(2-methylallyl) 1,5-cyclooctadiene complex, having CAS No. 12289-94-0.

“Dioxolane” as used herein refers to a chemical moiety of formula shown below and boxed in dotted lines within Compound I:

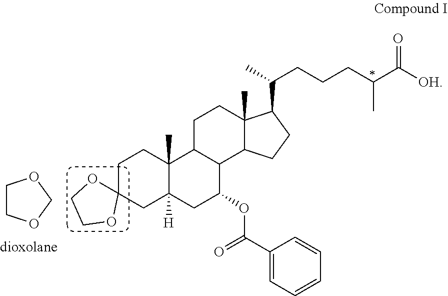

“Reductive amination” as used herein, refers to a synthetic procedure whereby a ketone or aldehyde is reacts with an amine to form in imine or iminium which is subsequently reduced to form an amine. Methods and conductions to affect reductive amination are known to those of ordinary skill in the art. See, for example, Dangerfield et al., J. Org. Chem., 2010, 75, 5470-5477; Taibakhsh et al., Synthesis, 2011, 490-496; and Abdel-Magid et al., J. Org. Chem., 1996, 61, 3849-3862; the entire disclosures of which are hereby incorporated by reference.

Any references to the C₂₅ carbon of a steroid or intermediate to the synthesis thereof refers to the carbon marked “25” in Compound III and, for example, its synthetic precursors as shown below:

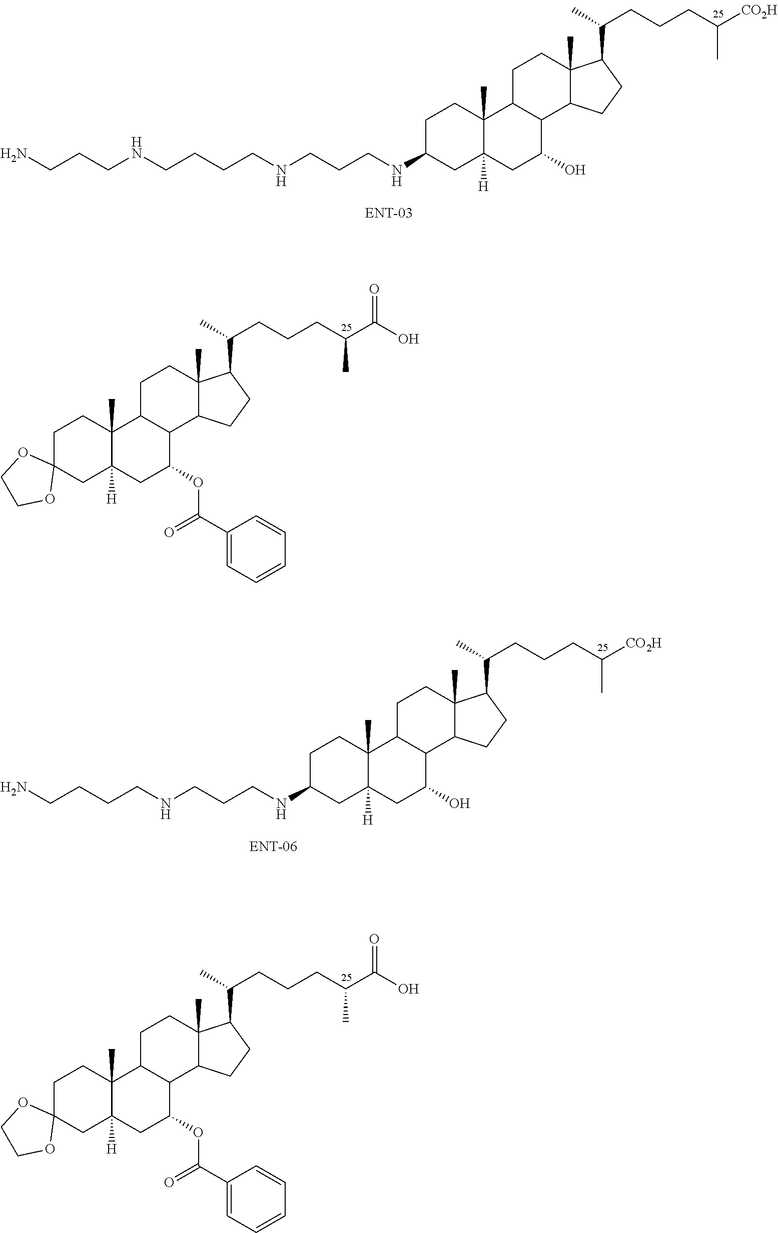 [SMILES: CC(CCC[C@@H](C)[C@H]1CCC2C3C(CC[C@@]21C)[C@@]1(C)CC[C@H](NCCCNCCCCNCCCN)C[C@@H]1C[C@H]3O)C(=O)O ; C[C@H](CCC[C@H](C)C(=O)O)[C@H]1CCC2C3C(CC[C@@]21C)[C@@]1(C)CCC2(C[C@@H]1C[C@H]3OC(=O)c1ccccc1)OCCO2 ; CC(CCC[C@@H](C)[C@H]1CCC2C3C(CC[C@@]21C)[C@@]1(C)CC[C@H](NCCCNCCCCN)C[C@@H]1C[C@H]3O)C(=O)O ; C[C@H](CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]

It is to be understood, that in any aminosterol or steroid compound disclosed herein, the stereochemical configuration of the 17 and 20 carbons are equally represented as depicted in the two example steroid nuclei shown below, i.e., the configurations of the 17 and 20 carbons, as drawn below, are the same:

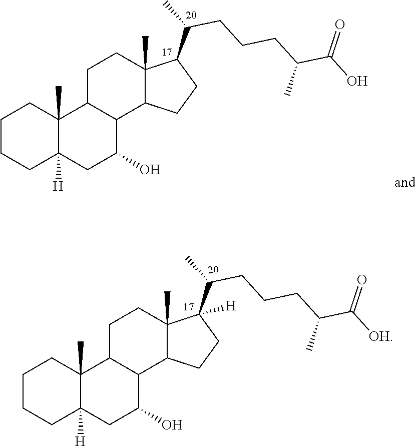 [SMILES: C[C@H](CCC[C@@H](C)[C@H]1CCC2C3C(CC[C@@]21C)[C@@]1(C)CCCC[C@@H]1C[C@H]3O)C(=O)O ; C[C@H](CCC[C@@H](C)[C@H]1CCC2C3C(CC[C@@]21C)[C@@]1(C)CCCC[C@@H]1C[C@H]3O)C(=O)O]

In any embodiment herein, wherein the ring carbons of the aminosterol (or corresponding carbons of synthetic intermediates corresponding to Compound I-R or -S) marked with exclamation points below are not stereodefined, embodiments wherein those carbons are defined as shown in Formula II are within the scope of this invention:

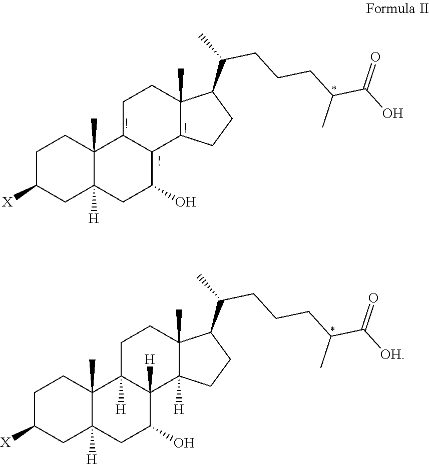

Embodiments described herein are further illustrated by, though in no way limited to, the following working examples.

### EXAMPLES

### Example 1: Synthesis of ENT-03 and ENT-06 C₂₅ R and S Isomers

This example describes synthetic methods of making compounds described herein.

### Preparation of BDG-5

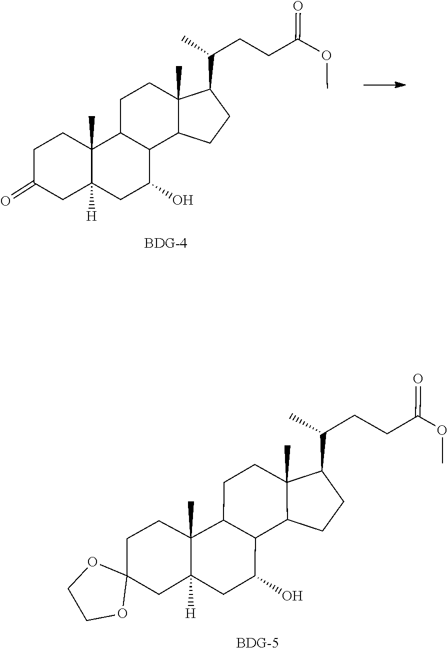 [SMILES: COC(=O)CC[C@@H](C)[C@H]1CCC2C3C(CC[C@@]21C)[C@@]1(C)CCC(=O)C[C@@H]1C[C@H]3O ; COC(=O)CC[C@@H](C)[C@H]1CCC2C3C(CC[C@@]21C)[C@@]1(C)CCC2(C[C@@H]1C[C@H]3O)OCCO2]

BDG-4 (370.54 g, 916.3 mmol, commercially available from Bridge Organics) was dissolved in dichloromethane (DCM, 3,600 mL) and treated with ethylene glycol (210 mL, 3.77 mol, 4.11 eq.) along with p-toluenesulfonic acid-hydrate (36.27 mmol, 0.04 eq.). The mixture was refluxed until the reaction was complete (TLC in 70:30 hexane:acetone showed ca 3-5% residual starting material that did not change). After about 4 h reflux, the mixture was cooled, treated with of 10% potassium carbonate (150 mL), and the layers separated. The DCM was re-extracted with 800 mL of 5% sodium chloride solution and 10 mL of 10% potassium bicarbonate solution. The two aqueous layers were back-extracted with 500 mL of DCM. The combined DCM extracts were dried over magnesium sulfate, filtered, washed with DCM (2×100 mL), and concentrated under vacuum to afford a solid, which was slurried in acetonitrile (4.8 L) containing triethylamine (2 mL) at 55° C. for 1 h, then 35° C. for 1 hour, and finally at 12° C. for 2 h. The product was filtered and washed with cold acetonitrile (2×100 mL) containing triethylamine (0.1 mL). The crystals were dried at 50° C. in the vacuum oven (0.2 mm) overnight to afford BDG-5 (351.0 g, 85%); m.p. was 175.6-177.5° (at 2°/minute from ca 140°); ¹H NMR (500 MHz, CDCl₃) δ 3.93 (m, 4H), 3.82 (br s, 1H), 3.66 (s, 3H), 2.4-2.2 (m, 2H), 1.9-1.2 (m, 24H), 0.92 (d, 3H, J=7 Hz), 0.81 (s, 3H), 0.66 (s, 3H); ¹³C NMR (CDCl₃) δ 177, 109, 67.9, 64.2, 64.1, 55.8, 51.4, 50.5, 45.6, 42.6, 39.5, 39.4, 37.5, 36.3, 36.1, 35.7, 35.5, 35.4, 31.2, 31.0, 30.9, 28.0, 23.6, 20.9, 18.3, 11.8, 10.4.

### Preparation of BDG-6

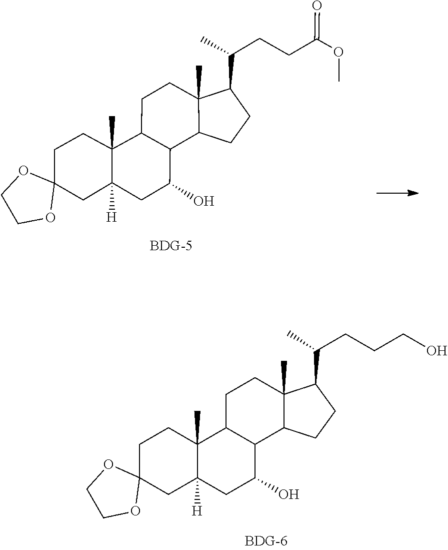 [SMILES: COC(=O)CC[C@@H](C)[C@H]1CCC2C3C(CC[C@@]21C)[C@@]1(C)CCC2(C[C@@H]1C[C@H]3O)OCCO2 ; C[C@H](CCCO)[C@H]1CCC2C3C(CC[C@@]21C)[C@@]1(C)CCC2(C[C@@H]1C[C@H]3O)OCCO2]

Lithium aluminum hydride (7.44 g, 196.05 mmol, pellets) was added to anhydrous THE (435 mL) under nitrogen and stirred overnight at 20° C. to break up the pellets. The suspension was cooled to 10° C. and a solution of BDG-5 (77.57 g, 172.91 mmol) was added dropwise over 160 min. The mixture was stirred 1 h further, and was quenched by adding 2 mL of ethyl acetate, followed by dropwise addition of 20% aqueous potassium hydroxide solution (7.44 mL) over 10 min. The mixture became very thick, then thinned out and became more granular with additional stirring for 15 h at 20° C. The mixture was filtered through Celite® 545 to remove the aluminum salts. The filter cake was washed with of THE (5×90 mL) to ensure that all the product was obtained in the filtrate, which was concentrated to afford crude BDG-6 (82.01 g, theory=72.7 g). In practice in larger runs, the THF solution was concentrated to dryness, and re-concentrated with DCM to remove traces of water prior to next step; ¹H NMR (500 MHz, CDCl₃) δ 3.93 (m, 4H), 3.82 (br s, 1H), 3.61 (m, 2H), 1.97-1.84 (m, 4H), 1.7-1.0 (m, 22H), 0.93 (d, 3H, J=7 Hz), 0.81 (s, 3H), 0.66 (s, 3H); ¹³C NMR (CDCl₃) δ 109.3, 67.9, 64.15, 64.13, 63.5, 56.0, 50.6, 45.6, 42.6, 39.51, 39.48, 37.5, 36.3, 36.1, 35.7, 35.6, 35.5, 31.8, 31.2, 29.4, 28.2, 23.6, 20.9, 18.6, 11.8, 10.4.

### Preparation of BDG-7

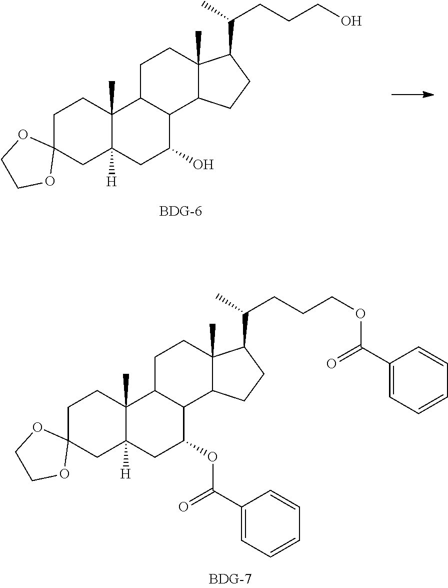 [SMILES: C[C@H](CCCO)[C@H]1CCC2C3C(CC[C@@]21C)[C@@]1(C)CCC2(C[C@@H]1C[C@H]3O)OCCO2 ; C[C@H](CCCOC(=O)c1ccccc1)[C@H]1CCC2C3C(CC[C@@]21C)[C@@]1(C)CCC2(C[C@@H]1C[C@H]3OC(=O)c1ccccc1)OCCO2]

BDG-6 (214.55 g, 510.1 mmol) was dissolved in DCM (3.0 L) and treated with 4-dimethylaminopyridine (250.3 g, 2.048 mol) and then benzoyl chloride (180 mL, 217.98 g, 1.551 mol) dropwise. An exotherm to 31° C. was noted after about 40 mL of benzoyl chloride was added. The temperature was held to 25° C. for the remainder of the addition which took a total of 35 min. The solution was stirred overnight at 25° C., diluted with DCM (200 mL), and treated with 10% aqueous potassium bicarbonate (2 L) generating a small amount of carbon dioxide. The layers were separated and the aqueous layer was re-extracted with DCM (500 mL). The total organic layers were dried over magnesium sulfate and concentrated to afford BDG-7 as a solid (163.6 g, 100%). In practice in larger runs, the dibenzoate solution was concentrated to a suitable volume for the next step. ¹H NMR (500 MHz, CDCl₃) δ 8.07 (d, 2H, J=8 Hz), 8.00 (d, 2H, J=8 Hz), 7.57 (t, 1H, J=8 Hz), 7.53 (t, 1H, J=8 Hz), 7.47 (t, 2H, J=8 Hz), 7.41 (t, 2H, J=8 Hz), 5.16 (br s, 1H), 4.25 (m, 2H), 3.87 (m, 4H), 2.02-1.85 (m, 2H), 1.8-1.1 (m, 22H), 0.95 (d, 3H, J=7 Hz), 0.88 (s, 3H), 0.68 (s, 3H); ¹³C NMR (CDCl₃) δ 166.7, 166.0, 132.8, 131.1, 130.5, 129.7, 129.5, 128.4, 128.3, 109.0, 71.9, 65.5, 64.2, 64.1, 55.8, 50.7, 47.2, 42.8, 39.5, 38.6, 37.4, 37.2, 35.7, 35.5, 35.3, 33.3, 32.0, 31.2, 28.0, 25.2, 23.6, 21.1, 18.6, 11.8, 10.5.

### Preparation of BDG-8

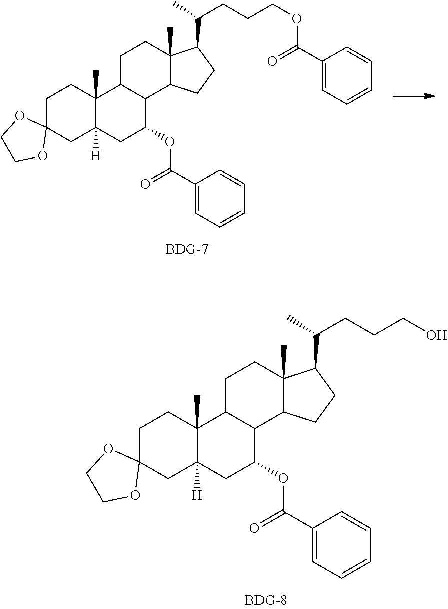 [SMILES: C[C@H](CCCOC(=O)c1ccccc1)[C@H]1CCC2C3C(CC[C@@]21C)[C@@]1(C)CCC2(C[C@@H]1C[C@H]3OC(=O)c1ccccc1)OCCO2 ; C[C@H](CCCO)[C@H]1CCC2C3C(CC[C@@]21C)[C@@]1(C)CCC2(C[C@@H]1C[C@H]3OC(=O)c1ccccc1)OCCO2]

BDG-7 (22.13 kg, 35.2 mol) was dissolved in 1:1 tetrahydrofuran:methanol solution (220 L) at 25° C. and treated with a solution of 50% sodium hydroxide (3.7 L, 70.4 mol, 2.0 eq.) in deionized water (11 L) and stirred for 4 h. An aliquot (0.1 mL) of the reaction mixture was partitioned between ethyl acetate (0.5 mL each) and 1 M potassium bicarbonate solution. The organic layer was analyzed by TLC (70:30 hexane:acetone) and the reaction was judged complete. An aqueous solution of potassium bisulfate was prepared by mixing deionized water (23 L), 96% sulfuric acid (3.7 L, 66.6 moles), and 45% potassium hydroxide (5.7 L, 66.7 moles). The resulting solution of potassium bisulfate was added via spray ball to the reaction mixture to a final pH of 8.39 (pH meter). The mixture was vacuum-distilled to about 60 L volume; and treated with of ethyl acetate (100 L) and water (100 L). The 2-phase mixture was agitated for 10 min, and the layers separated over about 20 min. The aqueous phase was re-extracted with 37 L of ethyl acetate. The total ethyl acetate layer (about 160 L) was vacuum concentrated to a volume of 80 L at 50° C. Another 50 L of ethyl acetate was added, and the solution dried over anhydrous sodium sulfate (15 kg) by stirring overnight. The solution was filtered over Celite® 545 to remove the sodium sulfate. The filter cake and the reactor were rinsed with 2×37 L of ethyl acetate. The dried ethyl acetate solution was vacuum concentrated from about 225 L volume to 40 L. Acetonitrile (50 L) was added and the solution was re-distilled to remove ethyl acetate. A second and third batch of acetonitrile (50 and 100L) were added and evaporated. Finally, 100 L of acetonitrile was added and this solution was counter-currently extracted with 3×104 L of hexane to remove most of the methyl benzoate. The 3 hexane extracts were re-extracted with a second 104 L of acetonitrile.

The hexane layers were checked by TLC for BDG-8 intermediate and none was present. The acetonitrile (ca. 300 L) was vacuum concentrated to ca. 50 L volume. Isopropanol (57 L) was added and concentrated to ca. 50.L; this operation was repeated 2 more times to remove all the acetonitrile. The volume was adjusted to 100 L with isopropanol and the solution slowly cooled to 0° C. and was seeded with crystalline BDG-8 to initiate crystallization. The mixture was slowly cooled to −20° C. overnight. The crystal slurry was filtered through a jacketed filter at −20° C. and rinsed with 10 L of −20° C. isopropanol. The filter cake was dried by vacuum for several h at −20° C., followed by letting the filter warm slowly to room temperature (about 18° C.) and then drying with warm nitrogen yielding 11.92 kg of BDG-8. The mother liquors were chromatographed to isolate additional product. A column of silica gel (19 kg) was packed in methylene chloride. The filtrate of the product was vacuum concentrated to dryness and 12 was added to the column in methylene chloride. Elution with 100 L of methylene chloride (DCM) gave the product. The column was flushed with 20 L of 85:15 methylene chloride:ethyl acetate, then 20 L of 60:40 methylene chloride:ethyl acetate, and finally back to methylene chloride (40 L).

The second half of the mother liquors was similarly chromatographed to give a total of 5.32 kg of pure BDG-8 (Total yield of BDG-8: 17.24 kg, 93.3%). ¹H NMR (500 MHz, CDCl₃) δ 8.07 (d, 2H, J=8 Hz), 7.57 (t, 1H, J=7 Hz), 7.48 (t, 2H, J=7 Hz), 5.15 (br s, 1H), 3.88 (m, 4H), 3.56 (br s, 2H), 2.02-1.84 (m, 2H), 1.75-0.98 (m, 24H), 0.92 (d, 3H, J=6.5 Hz), 0.88 (s, 3H), 0.67 (s, 3H). ¹³C NMR (CDCl₃) δ 166.0, 132.7, 131.0, 129.7, 128.4, 109.0, 71.9, 64.2, 64.1, 63.5, 55.8, 50.7, 47.2, 42.7, 39.5, 38.6, 37.3, 37.2 35.7, 35.5, 33.3, 31.7, 31.3, 29.3, 28.0, 23.6, 21.1, 18.6, 11.8, 10.5.

### Preparation of Compound 1

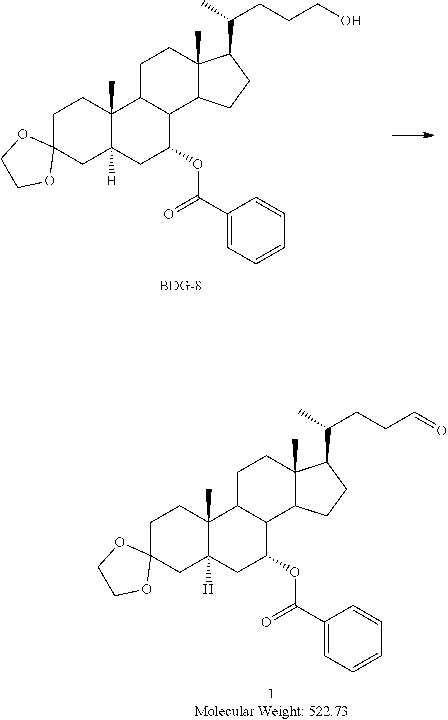 [SMILES: C[C@H](CCCO)[C@H]1CCC2C3C(CC[C@@]21C)[C@@]1(C)CCC2(C[C@@H]1C[C@H]3OC(=O)c1ccccc1)OCCO2 ; C[C@H](CCC=O)[C@H]1CCC2C3C(CC[C@@]21C)[C@@]1(C)CCC2(C[C@@H]1C[C@H]3OC(=O)c1ccccc1)OCCO2]

A 50 L jacketed reactor under nitrogen was charged with potassium bromide (250 g, 2.23 mol), sodium bicarbonate (265 g, 3.15 mol), and water (5 L); agitated and cooled with a −5° C. jacket. To the reactor was added BDG-8 (5.32 kg, 10.14 mol) dissolved in ˜13 L of dichloromethane, followed by rinse from 8 L of dichloromethane. When the reaction mixture attained a temperature <5° C., TEMPO (50 g, 0.286 mole) was added through the manhole. The reactor was then charged via pump with 12.5% bleach (5.5 kg), keeping the mixture <5° C. The reaction was sampled to estimate additional amount of bleach needed. Bleach continued to be added in small portions while sampling periodically until reaction was judged to be complete (<2% BDG-8 remaining; a total of 6.2 kg of bleach was added). A solution of sodium thiosulfate (200 g) in water (1 L) was added with vigorous stirring until a negative starch/iodide test was obtained. Hexane (20 L) was added to the reactor with stirring and the layers settled. The lower aqueous phase was drained, and the organic layer was washed with saturated aqueous potassium bicarbonate (5 L). The organic phase was vacuum filtered through a pad of sodium sulfate/silica gel (1 kg of each) in a sintered glass filter funnel and rinsed with hexane/dichloromethane (1/1), dichloromethane, and finally 5% methyl-t-butyl ether (MTBE) in dichloromethane. The combined filtrates were evaporated in two batches in a Buchi apparatus at 40° C. Hexane was added to each batch and evaporated again until a thick slurry formed. The solids were filtered, washed with hexane, and dried in a vacuum oven to afford 1 (4.58 kg). The filtrates and washings were combined and concentrated to get a second crop, which was filtered and washed with 5% MTBE/hexane, then hexane, and dry to get another 565 g of product for a total yield of 5.145 kg (97% yield) of 1.

The material contained about 1.5% of residual starting material, but no detectable (NMR) carboxylic acid. ¹H NMR (500 MHz, CDCl₃) δ 9.72 (s, 1H), 8.07 (d, 2H, J=7 Hz), 7.59 (t, 1H, J=7 Hz), 7.49 (t, 2H, J=7 Hz), 5.16 (br s, 1H), 3.88 (m, 4H), 2.45-2.27 (m, 2H), 2.00-1.85 (m, 2H), 1.78-1.17 (m, 22H), 0.913 (d, 3H, J=Hz), 0.88 (s, 3H), 0.68 (s, 3H). ¹³C NMR (CDCl₃) δ 203, 166, 132.8, 131.0, 129.8, 128.4, 109, 72.0, 64.17, 64.11, 55.7, 50.7, 47.2, 42.8, 40.8, 39.5, 38.6, 37.3, 37.2, 35.7, 35.5, 35.4, 33.3, 31.2, 27.9, 27.8, 23.7, 21.1, 18.3, 11.7, 10.5.

### Preparation of Compound 2:

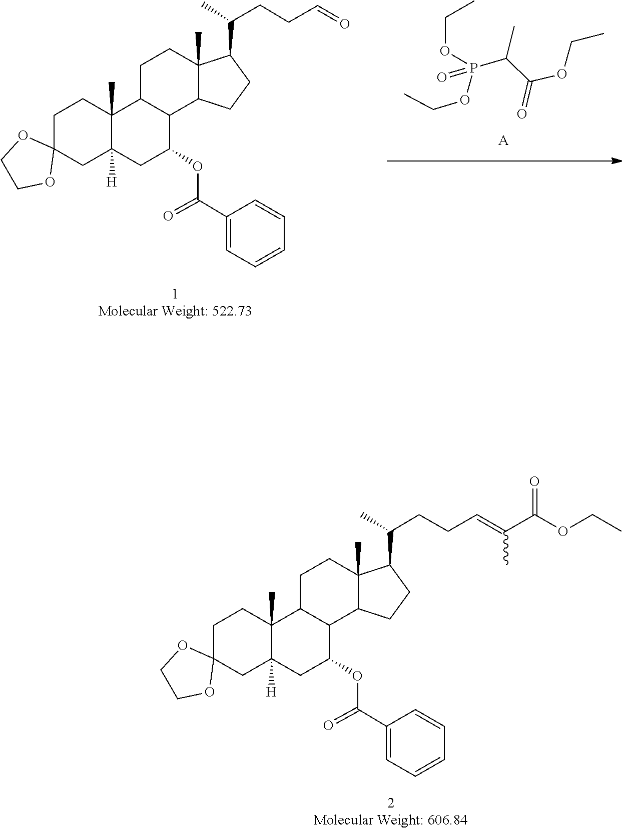

The phosphonate (A, 3.69 g, 15 mmol) was added to anhydrous tetrahydrofuran (100 mL) and chilled in a salt ice bath to −0° C. Potassium tert-butoxide (1.72 g, 15 mmol) was added with vigorous magnetic stirring under nitrogen and the reaction was allowed to stir for 30 min. Compound 1 (8.0 g, 15 mmol) was added and dissolved in tetrahydrofuran (80 mL), the ice bath was removed and the reaction was allowed to warm to RT overnight. The reaction after approximately 16 h total was worked up by partitioning between hexane/ethyl acetate (50/50, 400 mL) and water (400 mL). The organic layer was washed with an additional portion of water (100 mL) and the organic layer was dried over Na₂SO₄, filtered, and the solvent removed in vacuo. The residue was redissolved in a minimal amount of hexane/ethyl acetate 3/1 and passed through a plug of silica gel ˜3×9 in.

The eluant was then roto-evaporated to yield Compound 2 (8.3 g, 12.4 mmol, 83%) of satisfactory purity to utilize in the next step without further purification, ¹H NMR (CDCl₃, 300 MHz) δ 8.10-8.07 (m, 2H), 7.60-7.57 (m, 1H), 7.52-7.47 (m, 2H), 6.7, 5.9 (t, 1H), 5.17 (m, 1H), 4.21-4.12 (m, 2H), 3.92-3.88 (m, 4H), 2.06 (s, 3H), 2.2-1.0 (m, 29H), 0.95 (d, 3H, J=7 Hz), 0.90 (s, 3H), 0.69 (s, 3H); MS (ES+) 485.45 (M−C₇H₇O₂+H).

### Preparation of Compound 3

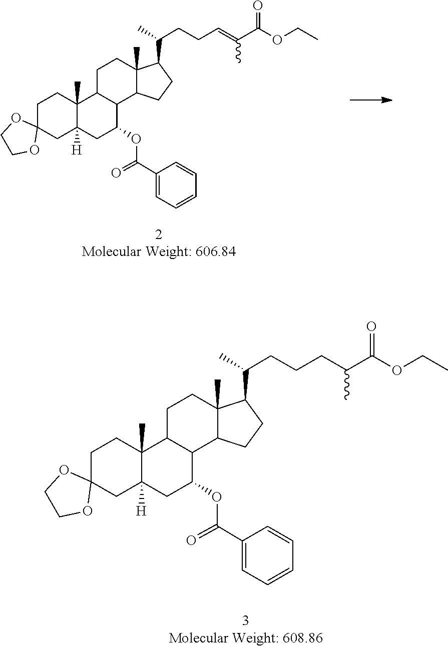

Compound 2 (8.25 g, 13.5 mmol) was dissolved in anhydrous ethanol and 10% Pd on C (400 mg) was added under N₂ in a Parr bottle (500 mL). The flask was flushed and filled 2× with vacuum and N₂ then hydrogenated at 50 psi for 24 h. The uptake of hydrogen had slowed to a near stop, but TLC showed a possible trace of starting material. An additional portion of catalyst (400 mg) was added, and the reaction was allowed an additional 12 h.

Filtration of catalyst and removal of the solvent in vacuo gave the saturated product in quantitative yield Compound 3 (8.25 g, 13.5 mmol), ¹H NMR (CDCl₃, 300 MHz) δ 8.09-8.06 (m, 2H), 7.58-7.56 (m, 1H), 7.55-7.45 (m, 2H), 5.16 (m, 1H), 4.12-4.05 (m, 2H), 3.90-3.85 (m, 4H), 2.39-2.36 (m, 1H), 2.0-1.0 (m, 35H), 1.11 (d, 3H, J=7 Hz), 0.88 (s, 3H), 0.67 (s, 3H); MS (ES+) 487.46 (M−C₇H₇O₂+H).

### Preparation of Compound 4

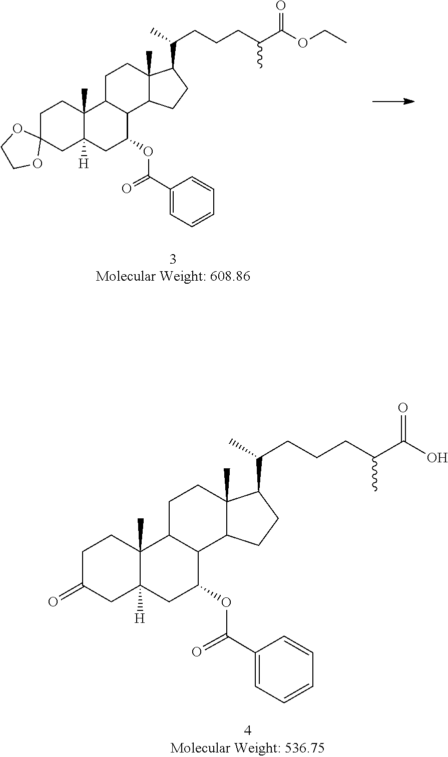

Compound 3 (8.2 g, 13.5 mmol) was dissolved in 3/1/1 tetrahydrofuran/methanol/1M KOH (˜100 mL) and stirred until hydrolysis of the ethyl ester appeared to be complete by TLC. There was no evidence of benzoate hydrolysis under these conditions. The solution was neutralized with 1 M hydrochloric acid solution, evaporated to remove the organic solvent, treated with acetone (˜100 mL), and evaporated again to ensure removal of any methanol. Acetone (˜250 mL) was added to the flask and 3M HCl was added to lower the pH to the point where it registered in the 1-2 range by pH paper. The hydrolysis of the ketal was carried out overnight at RT, water was then added to the flask, and the majority of the acetone was removed in vacuo. The material was partitioned between ethyl acetate and water, and then the organic layer washed with brine. The organic layer was dried in vacuo to give Compound 4 (6.36 g, 11.9 mmol, 87%) of satisfactory purity to be utilized without further purification, ¹H NMR (CDCl₃, 300 MHz) δ 8.05-8.02 (m, 2H), 7.60-7.57 (m, 1H), 7.51-7.45 (m, 2H), 5.21 (m, 1H), 2.4-1.0 (m, 32H), 1.15 (d, 3H, J=7 Hz), 1.09 (s, 3H), 0.91 (d, 3H, J=7 Hz), 0.67 (s, 3H); MS (ES+) 415.52 (M−C₇H₇O₂+H).

### Preparation of Compound 5

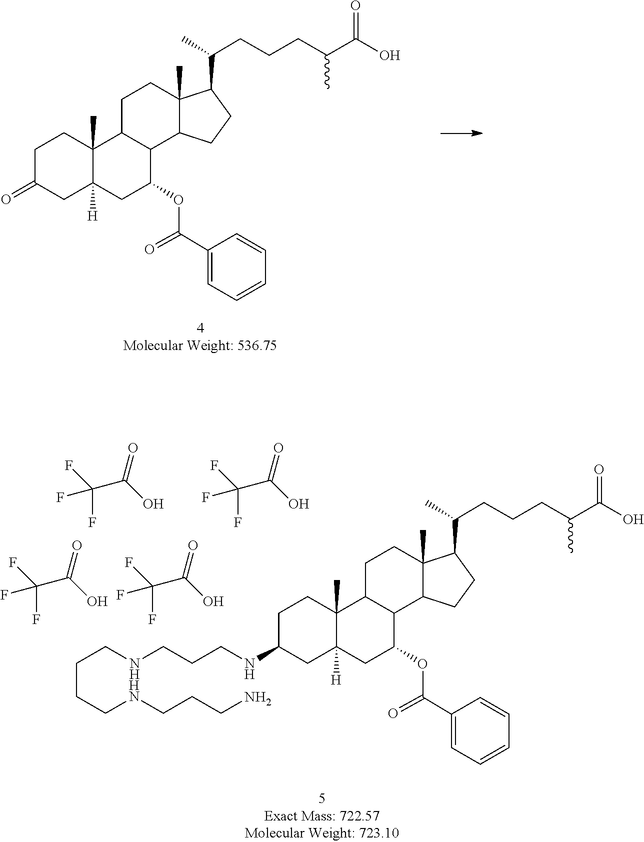

Compound 4 (3.5 g, 6.5 mmol) was dissolved in methanol (100 mL) and treated with spermine (5 g, 24.8 mmol) in methanol (˜10 mL). The mixture was stirred for 2 h at RT after which 2-propanol (100 mL) was added, and the majority of the solvent was removed in vacuo. The residue was redissolved in methanol (200 mL) and stirred overnight. Isopropyl alcohol (200 mL) was added and the mixture was evaporated to a thick residue. The residue was dissolved in anhydrous methanol (200 mL), and the solution chilled in a dry ice acetone bath under N₂ with vigorous magnetic stirring. When the internal temperature reached ˜−74° C., NaBH₄ (1.89 g, 50 mmol) was added. The temperature was maintained with the dry ice acetone bath for ˜4 h and then allowed to come to RT overnight. The reaction mixture was carefully acidified with 10% trifluoroacetic acid in water until pH paper showed pH 2-3 range. Water was added to the mixture, and the mixture was transferred to an oversized flask (to allow for frothing of the mixture on rotary evaporation) and the majority of the methanol removed in vacuo. The resulting solution was applied directly to amberchrome and eluted with a step gradient of acetonitrile in water with 0.5% TFA (10% increments 500 mL per increment) until aminosterol eluted (˜60% acetonitrile). The gradient was held at this point until all of the aminosterol eluted.

The fractions containing aminosterol were analyzed and the relatively clean fractions pooled and lyophilized to afford Compound 5 as the tetra-TFA salt (˜4.5 g, 3.8 mmol) of sufficient purity to carry on without further purification, ¹H NMR (CD₃OD, 300 MHz) δ 8.05-8.02 (m, 2H), 7.66-7.60 (m, 1H), 7.54-7.49 (m, 2H), 5.17 (m, 1H), 3.36-3.04 (m, 13H), 2.37 (m, 1H), 2.1-1.0 (m, 39H), 1.10 (d, 3H, J=7 Hz), 0.95 (m, 6H), 0.74 (s, 3H); MS (ES+) 723.78 (M+H).

### Preparation of ENT-03 (Compound III)

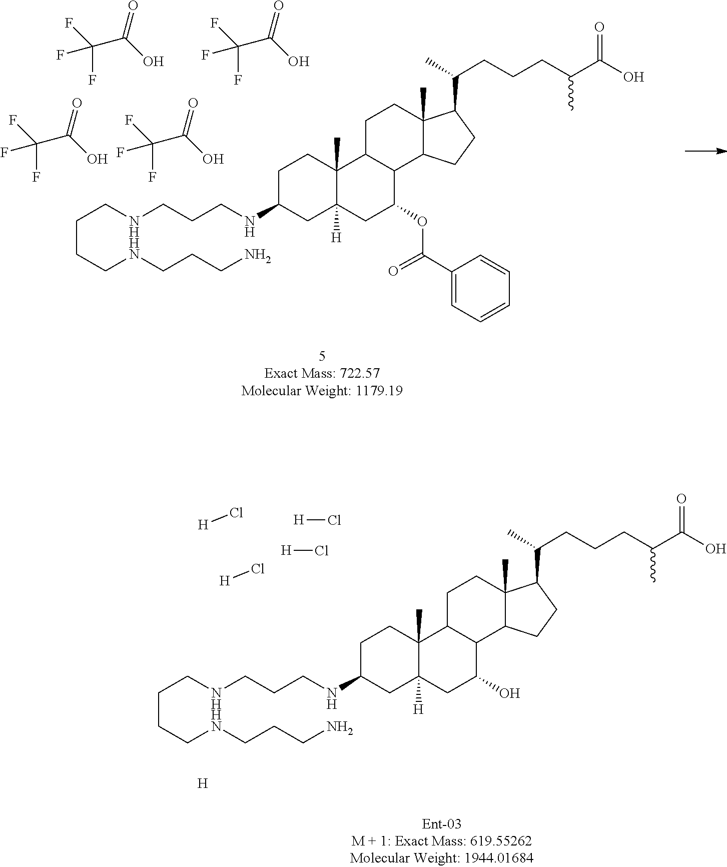

Compound 5 (3.0 g, 2.5 mmol) was added to of 5% methanolic potassium hydroxide (40 mL), and the solution was stirred for 2 days under N₂ at 110° C., and monitored with TLC (6:3:1 Chloroform, Methanol, conc. NH₄OH). After 2 days, the reaction was cooled to room temperature, evaporated under vacuum, and dissolved in H₂O (40 mL). This solution was then acidified with 6M HCl, and the white precipitate was forced back into solution with gentle heat and stirring. The solution was poured onto a large column of Amberchrome, and washed with H₂O (350 mL), increments (500 mL) of 10%, 15%, and 25% acetonitrile/water. Almost as soon as one column volume of 25% solution had passed through, the compound began to elute. The fractions (40 mL) that were collected were analyzed via LC/MS to separate away the 3-α side-product, which came off the column immediately following the desired 3-β product. Although there was some co-elution, a significant portion of the material came off cleanly.

These fractions were combined and lyophilized overnight to give (1.31 g, 1.7 mmol, 68%) of ENT-03 (Compound III) as the tetra-HCl salt, ¹H NMR (CD₃OD, 300 MHz) δ 3.80 (br s, 1H), 3.20-3.05 (m, 13H), 2.37 (m, 1H), 2.2-1.0 (m, 36H), 1.13 (d, 3H, J=7 Hz), 0.93 (d, 3H, J=7 Hz), 0.87 (s, 3H), 0.69 (s, 3H). ENT-03 was analyzed for purity via HPLC (Waters Acquity ELSD) under the following conditions: Mobile phase A: 0.1% formic acid in water; Mobile phase B: 0.1% formic acid in acetonitrile; Column: Kinetex XB-C18 (2.1×75 mm, 1.7 m); Gradient: 5-95%/8 min, hold 95% B/1 min; 0.6 mL/min flowrate; ELSD detector; Retention time: 1.96 min and 99.9% peak area. ENT-03 was analyzed by mass spectrometry (Waters Acquity TQD); Mobile phase A: 0.1% formic acid in water; Mobile phase B: 0.1% formic acid acetonitrile; Column: Kinetex XB-C18 (2.1×75 mm, 1.7 m); Gradient: 5-95%/8 min, hold 95% B/1 min; Flowrate: 0.6 ml/min; MS (ES+, M+H): Calc'd 619.55; Found: 619.31.

### Preparation of ENT-03S (Compound III-S)

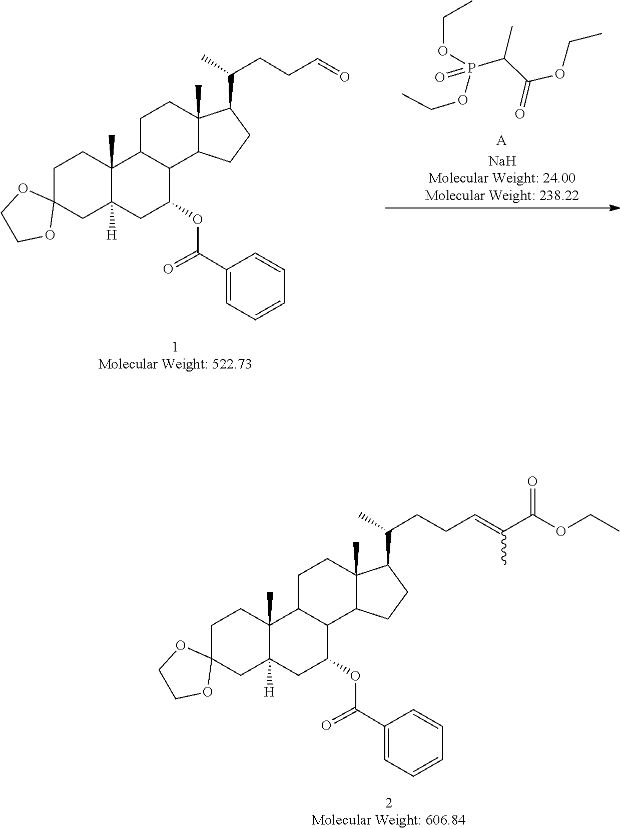

Tetrahydrofuran (THF, 10 mL) was added to 60% sodium hydride (330 mg, 8.25 mmol) in a 50 mL flask under nitrogen while cooling in an ice bath. Triethyl 2-phosphonopropionate (A, 1.31 g, 5.55 mmol) was added neat by syringe over 5 min so that gas evolution was controlled. The solution was stirred another 10 min after the addition ended. The substrate aldehyde 1 (2.613 g, 5.00 mmol) was dissolved in THE (4 mL) and this was added to the flask over 5 minutes, keeping the reaction temperature at 3-8° C. and washing in the residue with THF (0.5 mL) twice. The reaction was checked by thin layer chromatography (TLC) using 4:1 heptane:EtOAc and Hannesians stain. Conversion was complete on the first check 5 min after the addition. At 10 minutes, a saturated NH₄Cl (10 mL) solution was added at once with rapid stirring. The mixture was diluted with water until the solids dissolved. The aqueous phase was extracted with ethyl acetate (2×5 mL), and the combined organic phases were dried over Na₂SO₄ and concentrated to afford a gel (2.77 g). The TLC before and after workup shows an increase in the polar impurity spots. The crude was analyzed by LC-MS and ¹H-NMR. The E:Z ratio of the product was 5.0:1.0 by the NMR shifts at 6.68 and 5.84 ppm. The crude was purified by MPLC using a 25 g Biotage Snap column. The crude was loaded onto the column with DCM and the product was eluted with 0 to 35% EtOAc in hexanes over 12 column volumes. The top spot on the TLC was collected. The Rf of this spot was 0.65 in 4:1 4:1 heptane:EtOAc. The more polar spots present in the crude were removed by this purification to yield Compound 2 (1.99 g, 66%, E:Z ratio remained 5:1 after column) as a white foam. ¹H NMR (CDCl₃, 300 MHz) δ 8.12-8.03 (m, 2H), 7.64-7.53 (m, 1H), 7.53-7.42 (m, 2H), 6.70 (td, J=7.4, 1.6 Hz, 0.8H), 5.8 (t, 0.2H), 5.16 (m, 1H), 4.16 (q, J=7.1 Hz, 2H), 3.93-3-83 (m, 4H), 2.2-1.0 (m, 29H), 1.27 (s, 3H), 0.98-0.85 (m, 6H), and 0.68 (s, 3H); MS (ES+, M+Na): Calc'd: 629.38; Found: 629.77.

### Preparation of Compound 22

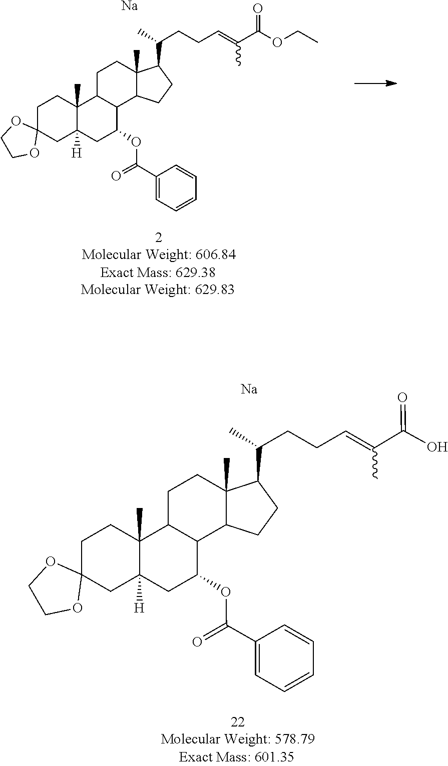

Compound 2 (1.43 g, 2.36 mmol) was combined with methanol (12.4 mL), tetrahydrofuran (12.4 mL), and 3 M NaOH in water (5.76 mL, 17.3 mmol) in a 50 mL flask with a septum and stir bar. The homogeneous mixture was stirred at 50° C. and monitored by LC-MS at 220 nm. At 90 minutes, the area percent of the desired product was 87 percent. 2M citric acid was added until the pH was 3.5, and the THF and MeOH were removed by rotavap at 30 degrees. The aqueous layer was extracted with ethyl acetate (3×20 mL) and the combined organic phase was dried with Na₂SO₄ and concentrated under vacuum to afford an amorphous white solid (1.488 g). The crude material was purified by Biotage MPLC with a 50 g Snap Ultra column. The product was loaded with a minimal amount of DCM and eluted with a gradient of 0 to 50% EtOAc in hexanes using 20 column volumes. Peaks were collected with a 254 nm detection threshold of 20 mAU. Partial separation of (E) and (Z)-olefins was seen in the fractions. The earlier fractions were combined and analyzed by 1H-NMR. These were enriched for the (Z) isomer as seen by the peak at 6.0 ppm. This was combined with the other product-containing fractions to give Compound 22 as a white foam (930 mg, 68%) of 10:1 E:Z ratio. ¹H NMR (CDCl₃, 300 MHz) δ 8.12-8.03 (m, 2H), 7.64-7.53 (m, 1H), 7.48 (ddt, J=8.4, 6.7, 1.3 Hz, 2H), 6.84 (td, J=7.5, 1.6 Hz, 1H), 5.16 (m, 1H), 4.27-4.11 (m, 1H), 3.98-3.81 (m, 4H), 2.3-1.0 (m, 26H), 1.80 (s, 3H), 0.92 (d, 3H), 0.88 (s, 3H) and 0.68 (s, 3H); MS (ES+, M+Na): Calc'd: 601.35; Found: 601.77.

### Preparation of Compound I-S, (S)-23

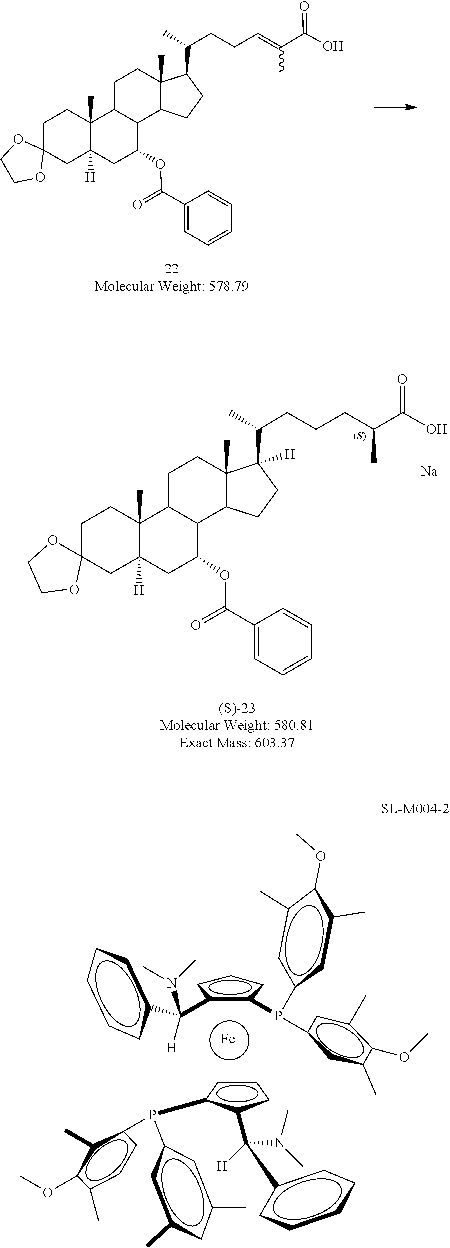

In the glovebox, (Me-Allyl)2Ru(COD) (93.66 mg, 0.293 mmol) and SL-M004-2 (308.74 mg, 0.293 mmol) were added to a 40 mL vial with a septum cap and stir bar. After adding isopropyl acetate (36.17 mL) with rapid stirring at room temperature, a solution of 48% HBr (72.97 μL, 0.645 mmol) in water was added dropwise and the mixture was stirred for 1 h in the glovebox. Compound 22 (3.965 g, 6.85 mmol) was added to each reactor of the HEL instrument in air as a solid. The reactors were sealed and purged with 200 psi of nitrogen 6 times, then with 200 psi of hydrogen 6 times. The orange catalyst solution was removed from the glovebox, the septum was fitted with a nitrogen balloon, and the catalyst solution (17.13 mL, 0.137 mmol) was injected into each purged reactor using a syringe with a 10 inch needle. The ports were shut, and the reactions were pressurized with hydrogen and heated to the desired temperature and pressure with 1000 RPM stirring. Hydrogen uptake was monitored to judge reaction completion. A blank of 21 mL of iPrOAc was subjected to the same program in another reactor to gauge hydrogen uptake by the solvent. Upon completion, the reactions were analyzed by UPLC (Table 4) and chiral HPLC to determine conversion, area percent (AP) of product S-23, and de of (S)-23 (Table 5). Excellent conversion and stereocontrol was observed under both conditions studied (Table 2) in delivering Compound (S)-23. ¹H NMR (CDCl₃, 300 MHz) δ 8.06 (m, 2H), 7.6-7.4 (m, 3H), 5.15 (m, 1H), 3.88 (m, 4H), 2.42 (m, 1H), 2.4-1.0 (m, 31H), 0.88 (m, 6H) and 0.66 (s, 3H).

TABLE 2
Conditions and LC results of the pressure and
temperature study on a 6.85 mmol scale for the
hydrogenation of Compound 22 to Compound (S)-23.

| | Conditions^{a} | | | LC Results | | | |
|---|---|---|---|---|---|---|---|
| | Pressure | Temp. | Time | % | AP | % de | Σ AP |
| Reaction # | (psi) | (° C.) | (h)^{d} | Conv.^{b} | 23^{b} | (S)-23^{c} | Unkn.^{b} |
| R1 830-116-R2 | 80 | 50 | 48 | 100 | 83 | 93.5 | 17 |
| R2 830-116-R3 | 150 | 30 | 48 | 100 | 82 | 95.2 | 18 |

^{a}Conditions: 6.85 mmol 22 (≥27.6:1 E:Z) in 16.9 mL i-PrOAc with catalyst, 1000 rpm, 30° C. for R1 or 50° C. for R2. AP of substrate 22 was 87.8 by the achiral UPLC method.
^{b}Determined by the achiral UPLC method.
^{c}Determined by the chiral UPLC method.

### Preparation of Compound I-R, (R)-23

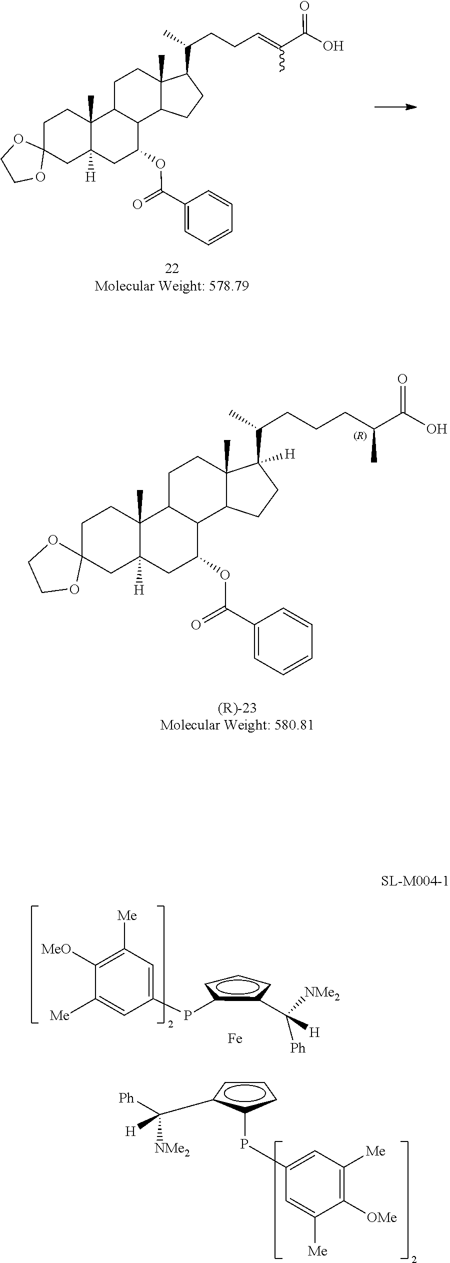

In the glovebox, SL-M004-1 (111.2 mg, 0.1056 mmol) was added to a 20 mL screw-cap vial with a stir bar, followed by (Me-Allyl)2Ru(COD) (33.7 mg, 0.1056 mmol). In a separate 20 mL vial, 48 wt. % HBr (39.3 mg, 0.233 mmol) in water was combined with isopropyl acetate (14.51 mL), making a solution of 0.016 M HBr solution. The HBr solution (13.2 mL) was added to make a catalyst solution of 0.008 M theoretical concentration. This was stirred in the glovebox for 30 minutes at room temperature. In the Biotage Endeavor reactor, Compound 22 (695 mg, 1.2 mmol) of was added and the reactor was sealed. Using the Endeavor control software, the reaction vessels were purged 10 times with 50 psi of nitrogen, then 10 times with 50 psi hydrogen. The catalyst solution (3 mL) was injected into the reactions vessels that were purged with H₂ and that each contained Compound 22. The pre-programmed reaction operation was started, raising each reaction to the set temperature, then pressurizing with hydrogen to the set pressure for 16 h. The glass reaction vials were removed from the reactor and 30 L of the reaction liquid was diluted with 4 mL of MeCN, filtered and analyzed by UPLC and chiral HPLC by the same methods described in Tables 4 and 5. Excellent conversion and stereocontrol was observed under all conditions studied (Table 3) in delivering Compound (R)-23. ¹H NMR (CDCl₃, 300 MHz) δ 8.06 (m, 2H), 7.6-7.4 (m, 3H), 5.15 (m, 1H), 3.88 (m, 4H), 2.42 (m, 1H), 2.4-1.0 (m, 31H), 0.88 (in, 6H), and 0.66 (s, 3H).

TABLE 3
A comparison of temperature, hydrogen pressure, and substrate impurity profile
in the hydrogenation of 22 to give (R)-23.

| | Reaction conditions | | | Substrate LC data^{d} | | | | | Reaction LC data^{d} | | | | |
|---|---|---|---|---|---|---|---|---|---|---|---|---|---|
| | T | P | Cat. | | 22 E:Z | AP imp | AP imp | | % | AP imp | Σ AP | % de | AP |
| Reaction #^{a} | (° C.) | (psi)^{b} | (mol %) | 22 source | ratio^{c} | RT 5.66 | RT 8.32 | AP 7 | Conv. | RT 5.66 | Unkn | 8 | 8 |
| RA 830- | 50 | 520 | 2 | 819-198-A | ≥25:1 | 2.4 | 1.3 | 95.5 | 100 | 3.1 | 5.7 | 94.4 | 91.2 |
| 073-A | | | | | | | | | | | | | |
| RB 830- | 50 | 115 | 2 | | | | | | 100 | 3.1 | 6.3 | 94.4 | 90.6 |
| 073-B | | | | | | | | | | | | | |
| RC 830- | 35 | 520 | 2 | | | | | | 100 | 3.1 | 6.0 | 94.7 | 90.9 |
| 073-C | | | | | | | | | | | | | |
| RD 830- | 70 | 450 | 2 | | | | | | 100 | 3.2 | 4.9 | 92.6 | 91.9 |
| 073-D | | | | | | | | | | | | | |
| RE 830- | 50 | 450 | 2 | | | | | | 100 | 3.1 | 6.9 | 93.9 | 90.0 |
| 073-F | | | | | | | | | | | | | |
| RF 830- | 50 | 450 | 2 | 819-198-B | ≥25:1 | 0.2 | 3.3 | 96.3 | 100 | 0.3 | 5.8 | 90.6 | 93.9 |
| 073-G | | | | | | | | | | | | | |
| RG 830- | 50 | 450 | 2 | 819-201-C | ≥30:1 | 0 | 2.2 | 94 | 99.6 | 0 | 6.3 | 92.2 | 93.3 |
| 073-H | | | | | | | | | | | | | |
| RH 830- | 50 | 500 | 0.4 | 830-055 | ≥20:1 | 0 | 0 | 98.4 | 100 | 0 | 2.1 | 94 | 97.9 |
| 057-C5 | | | | | | | | | | | | | |

^{a}RH was done on a 10 μmol scale. All others were done on a 1.2 mmol scale.
^{b}Actual readings are shown.
^{c}E:Z ratio was determined by proton NMR.
TBD = to be determined.
^{d}APs were measured at 238 nm.

TABLE 4
HPLC conditions for analysis of area percent
of acid 23 from asymmetric hydrogenation screen.
Method: acid hydrogenation
Column: Agilent Poroshell 120 EC-C18,
2.7 μm, 4.6 × 100 mm
Detector: UV 238 nm
MPA: H₂O with 0.1% TFA, MPB: MeCN with 0.1% TFA
T(column): 40° C.

| Time | Flow rate | |
|---|---|---|
| (min) | (mL/min) | B % |
| 0 | 1 | 70.0 |
| 10.0 | 1 | 95.0 |
| 12.0 | 1 | 95.0 |
| 12.1 | 1 | 70.0 |
| 16 | 1 | 70.0 |

TABLE 5
HPLC conditions for analysis of diastereomeric ratio of acid 23.

| | |
|---|---|
| Column Temperature: 30° C. | Injection Volume: 5 μL |
| Detector: 238 nm | Flow Rate: 0.7 mL/min |
| MPA: 05% FA in water | MPB: 05% FA in MeCN |
| Sampler temperature: 25° C. | Column: Chiralcel OJ-RH, 5 μm, |
| | 150 × 4.6 mm, P#17724 |

Gradient Elution

| Time (min) | MPB (%) |
|---|---|
| 0 | 60% |
| 20 | 60% |

Generation of Compound (R)-24 for x-ray.

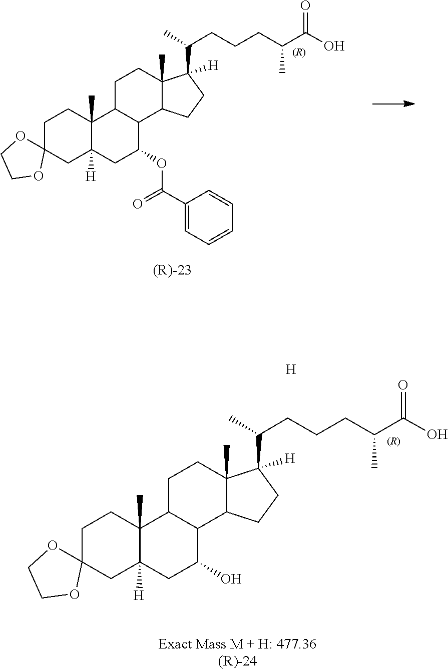

Compound (R)-23 (lot 830-050) was hydrolyzed in 5% KOH in methanol for 48 h. After acidified with half saturated citric acid solution in water, the crude product was extracted into ethyl acetate, dried over sodium sulfate, and evaporated. The material was dissolved in hot ethyl acetate, allowed to crystallize at room temperature, and collected by vacuum filtration. Large plate crystals of (R)-24 suitable for X-ray structure determination were grown by recrystallization of a sample (50 mg) in EtOAc. MS (ES+, M+H): Calc'd: 477.36; Found: 477.30; ¹H NMR (400 MHz, DMSO) δ 11.98 (s, 1H), 4.06 (d, J=4.0 Hz, 1H), 3.80 (s, 4H), 3.57 (t, J=3.2 Hz, 1H), 2.32-2.23 (m, 1H), 1.88 (ddq, J=12.6, 9.4, 3.3 Hz, 2H), 1.74 (ddt, J=12.9, 6.9, 3.0 Hz, 1H), 1.70-1.55 (m, 2H), 1.55-1.42 (m, 4H), 1.42-1.37 (m, 1H), 1.37-1.30 (m, 4H), 1.30-1.21 (m, 5H), 1.21-1.15 (m, 4H), 1.12 (dd, J=8.3, 3.8 Hz, 2H), 1.02 (d, J=6.9 Hz, 6H), 1.00-0.91 (m, 2H), 0.86 (d, J=6.5 Hz, 3H), 0.72 (s, 3H), 0.59 (s, 3H); ¹³C NMR (101 MHz, DMSO) δ 177.99, 108.96, 66.05, 63.98, 56.13, 50.52, 45.44, 42.40, 39.21, 37.91, 37.03, 35.98, 35.82, 35.76, 35.60, 35.53, 34.21, 31.41, 28.29, 23.54, 23.48, 21.07, 18.99, 17.38, 12.20, 10.67.

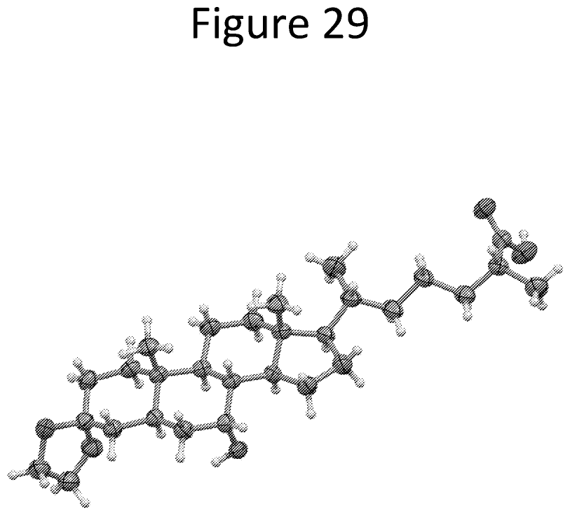

The X-Ray structure of (R)-24 was determined on a Bruker D8 QUEST Single-crystal X-ray Diffractometer, equipped with high brightness IpS 3.0 microfocus (50 kV×1 mA) for Cu radiation (λ=1.54178 Å) and with PHOTON II Charge-Integrating Pixel Array Detector of superior speed, sensitivity, and accuracy, was used for screening/evaluation of crystals and for diffraction data collection, as described below. The analogous S isomer may also be prepared and analyzed similarly from Compound I-S. Bruker APEX3 software suite including SHELXTL was used for diffraction experiments including data collection and integration, and for solving, refining, displaying, and publishing of structural results. A Cryostream 800 PLUS low temperature device was used. Keeping a crystal in a cold nitrogen gas stream prevents possible decay and reduces thermal motion of atoms and increases scattering power leading to better quality structures. A clear colorless plate-like crystal, approximate dimensions 0.020 mm×0.120 mm×0.260 mm, was used for the X-ray crystallographic analysis. The X-ray intensity data were measured (λ=1.54178 Å) at 223K. A total of 804 frames were collected. The total exposure time was 1.33 hours. The frames were integrated with the Bruker SAINT software package using a narrow-frame algorithm. The integration of the data using a monoclinic unit cell yielded a total of 34512 reflections to a maximum θ angle of 70.21° (0.82 Å resolution), of which 9503 were independent (average redundancy 3.632, completeness=96.9%, Rint=8.34%, Rsig=8.13%) and 6783 (71.38%) were greater than 2σ(F2). The final cell constants of a=11.2128(3) Å, b=11.1701(3) Å, c=21.0641(6) Å, β=91.458(2°), volume=2637.38(13) Å³, are based upon the refinement of the XYZ-centroids of 9993 reflections above 20 σ(I) with 8.398°<2θ<132.0°. Data were corrected for absorption effects using the Multi-Scan method (SADABS). The ratio of minimum to maximum apparent transmission was 0.639. The calculated minimum and maximum transmission coefficients (based on crystal size) are 0.8530 and 0.9870. The structure was solved and refined using the Bruker SHELXTL Software Package, using the space group P 1 21 1, with Z=4 for the formula unit, C29H4805. The final anisotropic full-matrix least-squares refinement on F2 with 630 variables converged at R1=5.52%, for the observed data and wR2=15.29% for all data. The goodness-of-fit was 1.035. The largest peak in the final difference electron density synthesis was 0.348 e⁻/Å³ and the largest hole was −0.246 e-/Å³ with an RMS deviation of 0.051 e⁻/Å³. On the basis of the final model, the calculated density was 1.200 g/cm³ and F(000), 1048 e⁻. Results confirmed assignment of the C-25 stereochemistry as the R-configuration. The absolute configuration (as shown below) was determined with single crystal diffraction data collected at 223K (see FIG. 29).

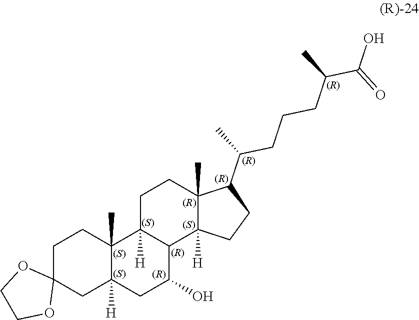

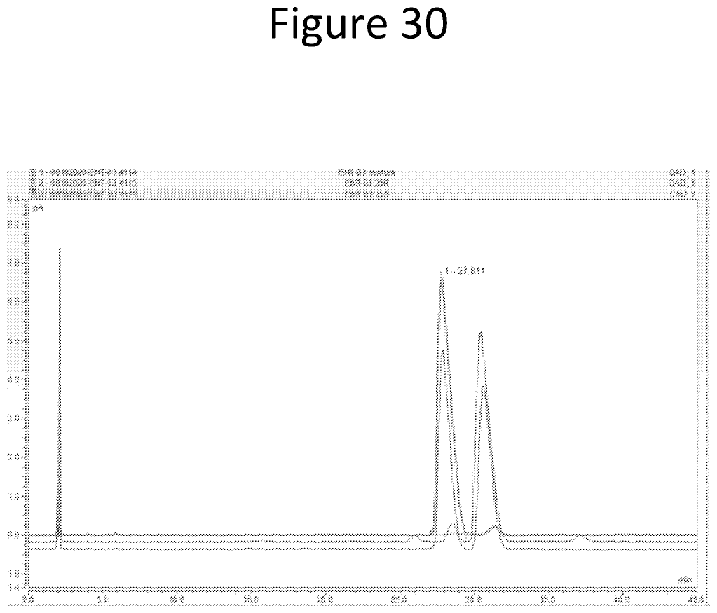

Compound (S)-23 (lot 830-051, de=96%) and (R)-23 (lot 830-050, de=90%) were converted to ENT-03S and ENT-03R, respectively, by the same means described above of the conversion of Compound 3 to ENT-03. ENT-03S and ENT-03R were analyzed by HPLC on a Kromasil, 100-5-C18, 5 μm, 250×4.6 mm, P #M05CLA25 column, using a Thermo Ultimate 3000 UPLC with CAD detector. The ¹H NMR and MS data were identical to those obtained for ENT-03. The samples were differentiated by HPLC as described in method below and depicted above in the image of (R)-24. The injection volumes of 1.0 to 2.0 μL appeared best without significantly sacrificing the separation. As can be seen in FIG. 30, baseline separation of the two isomers was achieved. The diastereomeric excess of these lots of ENT-03S and ENT-03R were determined to be 96% and 90%, respectively.

### Synthesis of Compound III-S and Compound III-R

Compound S-(23) and R-(23) was subjected to acetone (˜250 mL) was added to the flask and 3M HCl was added to lower the pH to the point where it registered in the 1-2 range by pH paper. The hydrolysis of the ketal was carried out overnight at RT, water was then added to the flask, and the majority of the acetone was removed in vacuo. The material was partitioned between ethyl acetate and water, and then the organic layer washed with brine. The organic layer was dried in vacuo to give the ketone which was subjected to the same conditions as described above in “Preparation of Compound 5” except spermine was used for preparation of C₂₅ stereodefined ENT-03 isomers or spermidine for C₂₅ stereodefined ENT-06 isomers, respectively.

### Preparation of ENT-03-d3

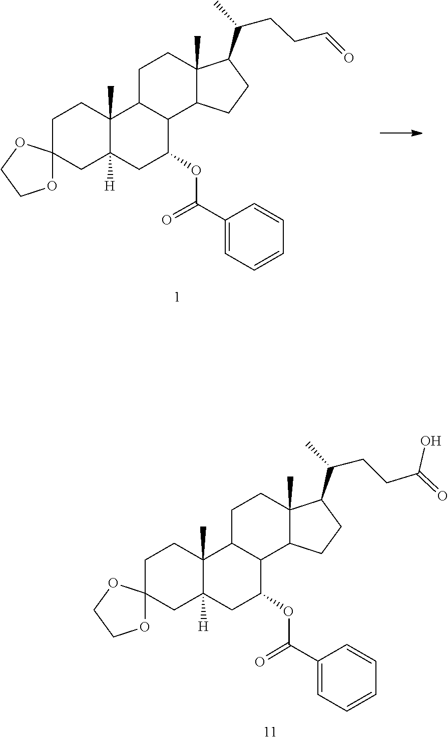 [SMILES: C[C@H](CCC=O)[C@H]1CCC2C3C(CC[C@@]21C)[C@@]1(C)CCC2(C[C@@H]1C[C@H]3OC(=O)c1ccccc1)OCCO2 ; C[C@H](CCC(=O)O)[C@H]1CCC2C3C(CC[C@@]21C)[C@@]1(C)CCC2(C[C@@H]1C[C@H]3OC(=O)c1ccccc1)OCCO2]

Compound 1 (2.6 g, 5 mmol) was dissolved in tetrahydrofuran (THF, 50 mL) and stirred over a sodium citrate/citric acid buffer ˜0.5 M (100 mL). Sodium chlorite (900 mg, 10 mmol) was dissolved in deionized (DI) water (20 mL) and added to the aldehyde solution with ice bath cooling. A slight yellow color was observed, and TLC (SiO₂, 15% EtOAc in hexane) shows a rapid conversion to the acid within 20 min. Ethyl acetate/hexane 50/50 (100 mL) was added, the aqueous removed, and the organic layer was washed with water, 10% thiosulfate solution, and brine to give the desired product 11 of satisfactory purity without further purification (2.7 g, 5 mmol, 100%).

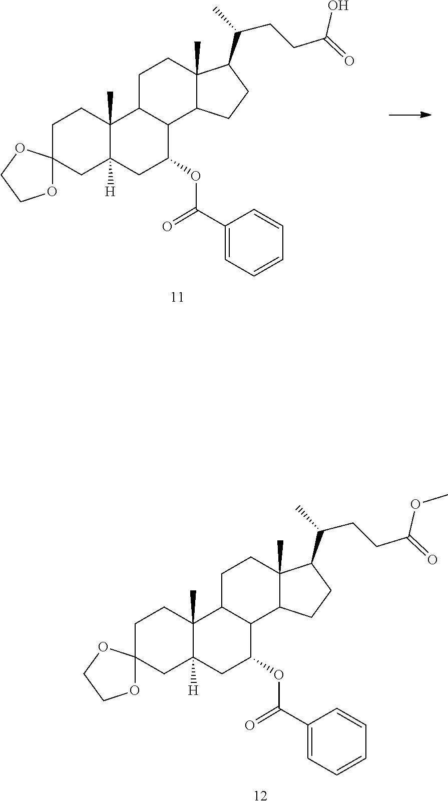 [SMILES: C[C@H](CCC(=O)O)[C@H]1CCC2C3C(CC[C@@]21C)[C@@]1(C)CCC2(C[C@@H]1C[C@H]3OC(=O)c1ccccc1)OCCO2 ; COC(=O)CC[C@@H](C)[C@H]1CCC2C3C(CC[C@@]21C)[C@@]1(C)CCC2(C[C@@H]1C[C@H]3OC(=O)c1ccccc1)OCCO2]

Compound 11 (2.6 g, 4.9 mmol) was dissolved in N-methylpyrrolidone (NMP, 25 mL) and anhydrous K₂CO₃ (˜2 g) was added and the reaction mixture was stirred under nitrogen for 20 min. Iodomethane (2.8 g, 20 mmol) was added in one portion and the reaction was stirred overnight under nitrogen. The reaction showed complete conversion to Compound 12 in nearly quantitative yield (2.6 g, 4.9 mmol).

Compound 12 (1.3 g, 2.3 mmol) was dissolved in anhydrous THE (30 mL) and anhydrous lithium chloride (˜1 g) was added. This solution was warmed to 40° C. and stirred under nitrogen. A solution of NaBD₄ in EtOD was prepared in 500 mg batches (2×3 mL) by dissolving the NaBD₄ in EtOD at room temperature and then chilling to prevent decomposition via the alkoxyborohydrides. The NaBD₄ solution was added in portions over time (4 h) and then warmed to 50° C. and stirred overnight under nitrogen. The reaction was diluted with water and extracted with 50/50 hexane/ethyl acetate. Organic layers were pooled dried over Na₂SO₄ and concentrated in vacuo. The residue was chromatographed on silica gel with 25% ethyl acetate in hexane to give the desired Compound 13 as a white solid (1.1 g, 2.0 mmol, 87%).

Compound 13 (1.0 g, 1.9 mmol) was dissolved in dichloromethane (50 mL), treated with activated 4 Å sieves (˜1 g) and 4-methylmorpholine N-oxide (500 mg, 4.3 mmol), and stirred 10 min at rt. Tetrapropylammonium perruthenate (˜40 mg) was added to the reaction and stirring was continued at rt overnight under nitrogen. This gave a single product matching the unlabeled Compound 1 by TLC. ¹H NMR did not show the aldehydic proton as expected.

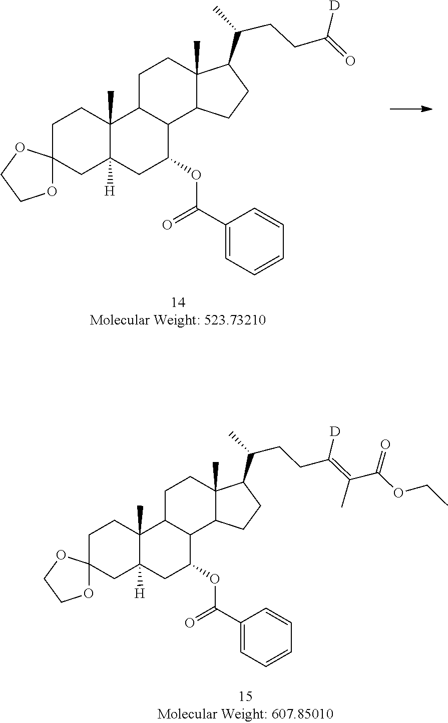 [SMILES: [2H]C(=O)CC[C@@H](C)[C@H]1CCC2C3C(CC[C@@]21C)[C@@]1(C)CCC2(C[C@@H]1C[C@H]3OC(=O)c1ccccc1)OCCO2 ; [2H]/C(CC[C@@H](C)[C@H]1CCC2C3C(CC[C@@]21C)[C@@]1(C)CCC2(C[C@@H]1C[C@H]3OC(=O)c1ccccc1)OCCO2)=C(/C)C(=O)OCC]

Triethyl phosphonopropionate (500 mg 2.0 mmol) was dissolved in EtOD and catalytic sodium ethoxide was added, stirred for 2 h, and the reaction mixture stripped to exchange the majority of the acidic methylene protons for deuterium. The phosphonate was added to anhydrous THE (30 mL) and treated with potassium t-butoxide (225 mg 2.0 mmol) at 0° C. for 30 min under nitrogen. The aldehyde 14 (1.0 g 1.9 mmol) was added in one portion at 0° C. in THE (˜5 mL) with rinses. The ice bath was removed, and the reaction mixture was allowed to run overnight at rt. The reaction mixture became slightly cloudy relatively quickly and is likely done in 1-2 h. The reaction mixture was partitioned between hexane/ethyl acetate 50/50 (˜100 mL) and water, and then washed with brine. The organic was dried over Na₂SO₄ and the solvent removed in vacuo. The relatively clean material was chromatographed on silica gel with a hexane ethyl acetate gradient to afford Compound 15 (860 mg, 1.4 mmol, 74%).

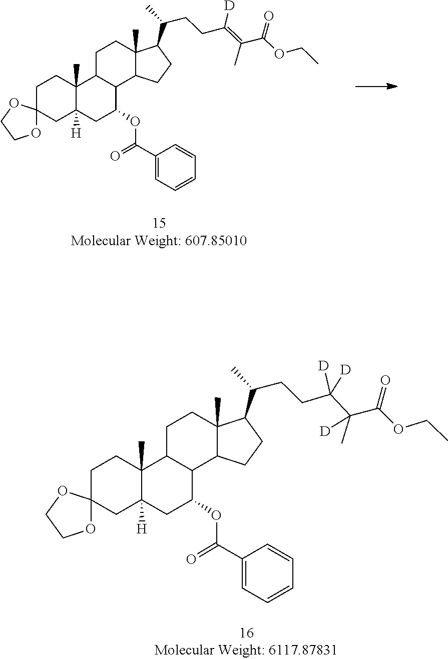 [SMILES: [2H]/C(CC[C@@H](C)[C@H]1CCC2C3C(CC[C@@]21C)[C@@]1(C)CCC2(C[C@@H]1C[C@H]3OC(=O)c1ccccc1)OCCO2)=C(/C)C(=O)OCC ; [2H]C([2H])(CC[C@@H](C)[C@H]1CCC2C3C(CC[C@@]21C)[C@@]1(C)CCC2(C[C@@H]1C[C@H]3OC(=O)c1ccccc1)OCCO2)C([2H])(C)C(=O)OCC]

The unsaturated ester 15 (860 mg, 1.4 mmol) was dissolved in ethyl acetate containing 20% EtOD and treated with the catalyst (10% Pd on Carbon, −100 mg) under nitrogen. The reaction was purged and backfilled with deuterium gas and then charged to 40 PSI in a 500 mL Parr shaker bottle. Agitation at room temperature overnight yielded a slight decrease in pressure. After an additional 8 h, no additional uptake was observed, so the reaction mixture was purged and backfilled with nitrogen three times and suction filtered through Celite®. The filtrate was evaporated in vacuo to give Compound 16 (850 mg, 1.4 mmol, 100%).

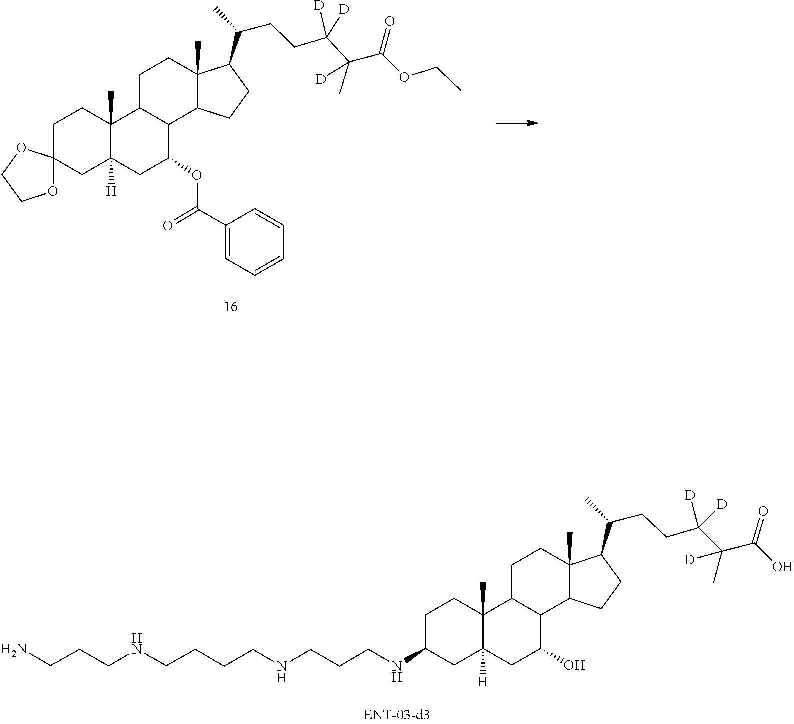 [SMILES: [2H]C([2H])(CC[C@@H](C)[C@H]1CCC2C3C(CC[C@@]21C)[C@@]1(C)CCC2(C[C@@H]1C[C@H]3OC(=O)c1ccccc1)OCCO2)C([2H])(C)C(=O)OCC ; [2H]C([2H])(CC[C@@H](C)[C@H]1CCC2C3C(CC[C@@]21C)[C@@]1(C)CC[C@H](NCCCNCCCCNCCCN)C[C@@H]1C[C@H]3O)C([2H])(C)C(=O)O]

Using the same procedures described for conversion of Compound 3 to ENT-03 (Compound III), Compound 16 was converted to ENT-03-d3. ¹H NMR (D₂O, 400 MHz) δ 3.90 (br s, 1H), 3.06-3.01 (m, 13H), 2.0-1.0 (m, 34H), 1.02 (s, 3H), 0.80 (br s, 3H), 0.73 (s, 3H), and 0.57 (s, 3H). ENT-03-d3 was analyzed for purity via HPLC (Agilent) under the following conditions: Mobile phase A: 0.1% formic acid in water; Mobile phase B: 0.1% formic acid in acetonitrile; Column: Kinetex XB-C18 (2.1×75 mm, 1.7 m); Gradient: 5-95%/8 min, hold 95% B; 0.6 ml/min flowrate; ELSD detector; Retention time: 4.93 and 95.5% peak area. ENT-03-d3 was analyzed by LC/MS (Waters Aquity HPLC-ZQ MS); Mobile phase A: 0.1% formic acid in water; Mobile phase B: acetonitrile; Column: Waters XBridge C18 (4.6×50 mm, 3.5 m). Flowrate: 1.1 mL/min. MS (ES+, M+H): Calc'd: 622.57; Found: 622.60.

### Preparation of ENT-03-d4

Compound 4 (2.14 g, 4.0 mmol) was dissolved in MeOD (50 mL) and 10% K₂CO₃ in D₂O was added (10 mL). The mixture was stirred under nitrogen at reflux for 16 h. The solvent was removed in vacuo and the residue was resuspended in MeOD (50 mL) and stirred at reflux for 8 h. The only change by TLC was the appearance of a small amount of unprotected C-7 hydroxyl, which was not a concern. The solvent was removed in vacuo and the Compound 21 was resuspended in MeOD (50 mL). Spermine (2.1 g, 10 mmol) was exchanged with MeOD (3×10 mL) by dissolving in the methanol stirring for 5 min. then removing the solvent in vacuo. A final portion of MeOD was added and the spermine solution was added to the exchanged sterol and the mixture was stirred overnight at room temperature. The solvent was removed in vacuo and the residue resuspended in MeOD (50 mL). This solution was stirred at rt for ˜9 h before chilling in a dry ice acetone bath for 40 min. Solid NaBH₄ (740 mg, 20 mmol) was added as a solid in two portions approximately 15 min apart. The bath was charged with dry ice and allowed to warm to rt overnight. Thin layer chromatograpy (TLC, SiO₂ 6/3/1 CHCl₃/MeOH/NH₄OH) of the reaction at ˜14 h reaction time showed no starting material and very little sign of 3-hydroxl of Compound 20. The reaction was diluted with deionized (DI) water and acidified slowly with trifluoro acetic acid (TFA) until strongly acidic by pH paper (pH<2). This gave a thick white precipitate, which was filtered, washed with 0.5% TFA in water, and then dissolved in 5% KOH in MeOH (100 mL) and refluxed overnight under nitrogen. After overnight reflux, the C7-benzoate hydrolysis was complete by TLC. The majority of the methanol was removed in vacuo and the material diluted with DI H₂O. The material was applied directly to an Amberchrome packed column (5×20 cm) and washed with water until the strongly basic portion had eluted. The column was then washed with 1% TFA and 5% acetonitrile in water until the eluant was acidic. A step gradient was run in 5% steps, and fractions containing the cleanest material by TLC pooled and the majority of the solvent removed in vacuo near the end of the solvent removal.

A solution (1 mL) of 6 N HCl was added and the solvent removed to exchange TFA for HCl. This was repeated three times and 2-propanol (100 mL) was added and quickly stripped off to give a free flowing white solid (700 mg, 0.96 mmol, 24% overall yield) in clean fractions of ENT-03-d4. ¹H NMR (D₂O, 400 MHz) δ 3.77 (br s, 1H), 3.06-2.99 (m, 13H), 2.36 (m, 1H), 2.0-1.0 (m, 33H), 1.05 (d, 3H), 0.84 (br s, 3H), 0.73 (s, 3H), and 0.57 (s, 3H). ENT-03-d4 was analyzed for purity via HPLC (Agilent) under the following conditions: Mobile phase A: 0.1% formic acid in water; Mobile phase B: 0.1% formic acid in acetonitrile; Column: Kinetex XB-C18 (2.1×75 mm, 1.7 m); Gradient: 5-95%/8 min, hold 95% B; 0.6 ml/min flowrate; ELSD detector; Retention time: 4.94 and 100% peak area. ENT-03-d4 was analyzed by LC/MS (Waters Aquity HPLC-ZQ MS); Mobile phase A: 0.1% formic acid in water; Mobile phase B: acetonitrile; Column: Waters XBridge C18 (4.6×50 mm, 3.5 m). Flowrate: 1.1 mL/min. MS (ES+, M+H): Calc'd: 623.58; Found: 623.31.

### Example 2: ENT-03 (Compound III) Activity as an Inhibitor of Protein Tyrosine Phosphatase 1B (PTP1B)

This example tested the PTP1B inhibitory activity of ENT-03 (Compound III) and ENT-02 (MSI-1436). Also included is comparative data of the inhibitory activity of D₇-1436 (D-1436), an aminosterol derivative having the structure:

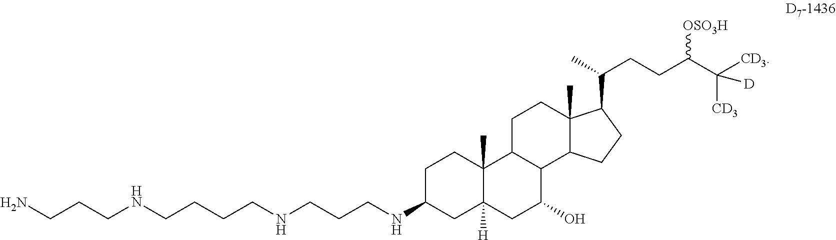

ENT-02 (MSI-1436), ENT-03 (Compound III), and D-1436 were dissolved in dimethyl sulfoxide (DMSO) to a stock concentration of 10 mM. A known PTP1B inhibitor, 3-(3,5-Dibromo-4-hydroxy-benzoyl)-2-ethyl-benzofuran-6-sulfonicacid-(4-(thiazol-2-ylsulfamyl)-phenyl)-amide (Wiesmann et al., 2004), served as a control. The compounds were tested in a 10-dose IC₅₀ mode with 3-fold serial dilution, in singlet, starting at 100 PM. The enzyme was the human truncated form (1-321), recombinantly produced in Escherichia coli. Fluorescence was measured to monitor enzyme activity. The phosphatase activities were monitored as a time-course measurement of the increase in fluorescence signal from fluorescent substrate, and initial linear portion of slope (signal/min) was analyzed. No compounds exhibited fluorescent background that could interfere with the assay.

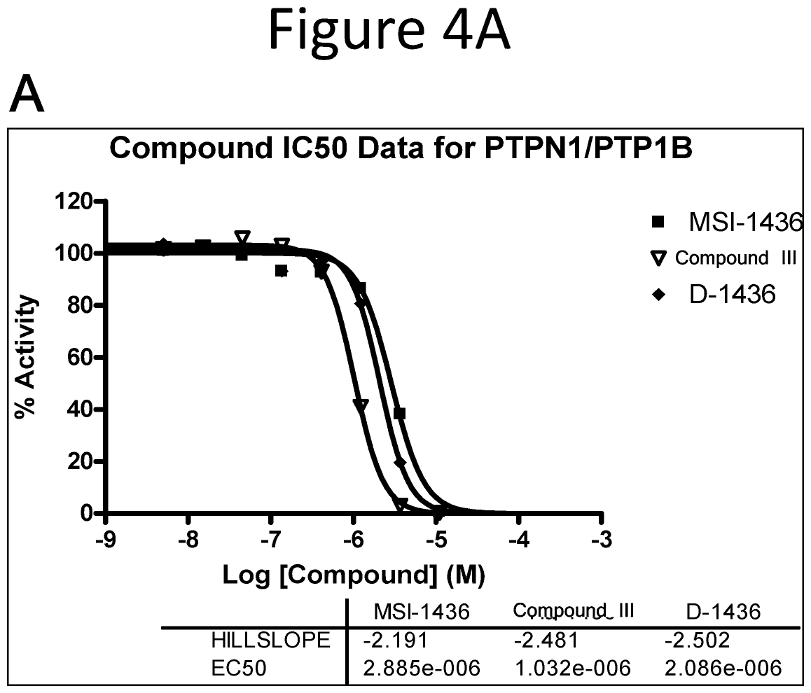

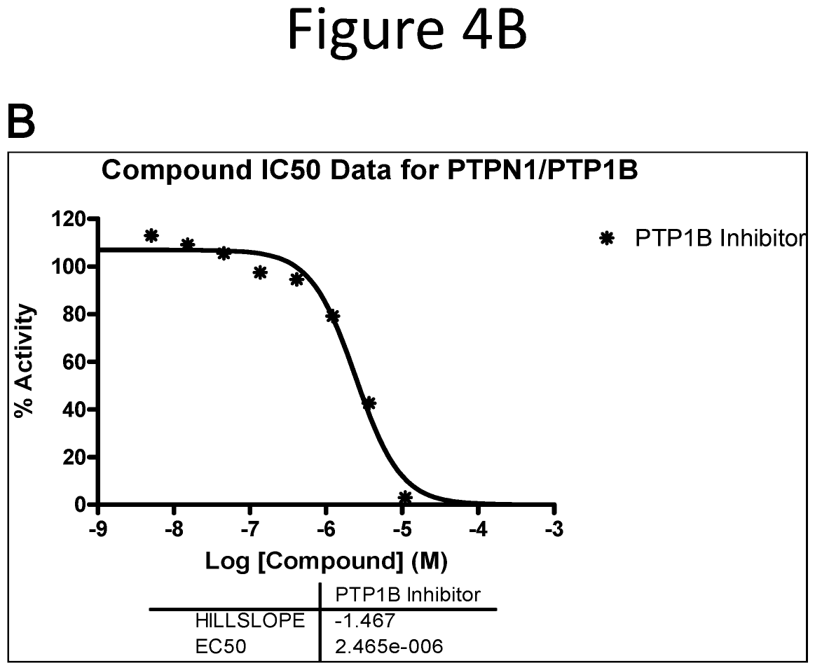

IC₅₀ curves were generated for the three aminosterols tested (FIG. 4A) and the control compound (FIG. 4B). Curve fits were performed when the activities at the highest concentration of compounds were less than 65%.

As seen in Table 5, ENT-02 (MSI-1436), already known to inhibit PTP1B, exhibited an IC₅₀ of 2.89 μM; ENT-03 (Compound III) exhibited an IC₅₀ of 1.03 μM; D-1436 exhibited an IC₅₀ of 2.09 μM; and the control PTP1B compound exhibited an IC₅₀ of 2.47 μM.

TABLE 5A
Phosphatase profiling report for: PTPN1/PTP1B

| | | | | |
|---|---|---|---|---|
| | ENT-02 | ENT-03 | PTP1B | |
| Conc (M) | (MSI-1436) | (Compound III) | Inhibitor | D₇-1436 |

Slope (signal/min)

| | | | | |
|---|---|---|---|---|
| 1.00E−04 | −68 | −334 | −810 | −161 |
| 3.33E−05 | −440 | −201 | −444 | −362 |
| 1.11E−05 | 292 | 109 | 1291 | −319 |
| 3.70E−06 | 16431 | 1266 | 18367 | 8429 |
| 1.23E−06 | 37260 | 17650 | 34136 | 34760 |
| 4.12E−07 | 39959 | 40062 | 40760 | 41755 |
| 1.37E−07 | 40137 | 44343 | 42024 | 40150 |
| 4.57E−08 | 42717 | 45535 | 45466 | 43697 |
| 1.52E−08 | 44021 | 44067 | 47019 | 43819 |
| 5.08E−09 | 43513 | 43877 | 48684 | 44655 |
| DMSO | 47697 | 44496 | 47769 | 47400 |

% Activity

| | | | | |
|---|---|---|---|---|
| 1.00E−04 | −0.16 | −0.77 | −1.88 | −0.37 |
| 3.33E−05 | −1.02 | −0.47 | −1.03 | −0.84 |
| 1.11E−05 | 0.68 | 0.25 | 2.99 | −0.74 |
| 3.70E−06 | 38.12 | 2.94 | 42.61 | 19.55 |
| 1.23E−06 | 86.43 | 40.94 | 79.19 | 80.63 |
| 4.12E−07 | 92.69 | 92.93 | 94.55 | 96.86 |
| 1.37E−07 | 93.11 | 102.86 | 97.48 | 93.14 |
| 4.57E−08 | 99.09 | 105.63 | 105.47 | 101.36 |
| 1.52E−08 | 102.11 | 102.22 | 109.07 | 101.65 |
| 5.08E−09 | 100.94 | 101.78 | 112.93 | 103.59 |
| DMSO | 110.64 | 103.22 | 110.81 | 109.95 |
| HILLSLOPE | −2.19 | −2.48 | −1.47 | −2.50 |
| IC₅₀ (M) | 2.89E−06 | 1.03E−06 | 2.47E−06 | 2.09E−06 |

The activity of ENT-03 and ENT-06 on a number of other phosphatases was also investigated. Both compounds were assayed in vitro against human phosphatases (Tables 2B and 2C). The two compounds exhibited a very similar phosphatase “fingerprint” with respect to their corresponding IC₅₀. These data strongly support the hypothesis that ENT-03 and trodusquemine are phylogenetic chemical orthologs. The compounds were found to be inactive at PP1A, PP1B, PP2 Å alpha, PTPN6, and PTPN2.

TABLE 5B
Activity of aminosterols on various phosphatases
Compound IC50 (M)

| | | | PP2A | | | |
|---|---|---|---|---|---|---|
| | | | Alpha/PP2R1A | PTPN6/ | PTPRC/ | DUSP22/ |
| Compound ID | PP1B | PP1A | Complex | SHP1 | CD45 | MKPX |
| ENT-02 (MSI- | >1.0E−04 | >1.0E−04 | 5.72E−05 | >1.0E−04 | 4.94E−07 | 6.39E−07 |
| 1436) | | | | | | |
| ENT-03 | >1.0E−04 | >1.0E−04 | >1.0E−04 | >1.0E−04 | 5.71E−07 | 1.38E−06 |
| (Compound III) | | | | | | |
| PTP1B Inhibitor | 3.46E−05 | 2.15E−05 | ND | 5.75E−06 | 5.70E−05 | 1.45E−05 |
| Canthardic Acid | ND | ND | 1.70E−08 | ND | ND | ND |
| SHP099 | ND | ND | ND | ND | ND | ND |

TABLE 5C
Activity of aminosterols on various phosphatases
Compound IC50 (M)

| | | | | PTPN1/ | | | PTPN11/ |
|---|---|---|---|---|---|---|---|
| | PTPN2/ | PTPN7/ | PTPN12/ | PTP1B- | PTPN11/ | PTPN11/ | SHP2- |
| Compound ID | TC-PTP | LC-PTP | PTP-PEST | CD | SHP2 | SHP2-FL | FL(E76K) |
| ENT-02 | >1.0E−04 | 1.65E−06 | 1.72E−06 | 9.44E−07 | 3.75E−07 | 7.52E−07 | 2.92E−07 |
| (MSI-1436) | | | | | | | |
| ENT-03 | | | | | | | |
| (Compound III) | >1.0E−04 | 6.34E−07 | 8.61E−07 | 1.17E−07 | 5.00E−06 | 1.42E−06 | 2.94E−07 |
| PTP1B | 1.34E−05 | 1.28E−05 | 2.99E−05 | 4.25E−06 | 1.82E−06 | ND | ND |
| Inhibitor | | | | | | | |
| Canthardic | ND | ND | ND | ND | ND | ND | ND |
| Acid | | | | | | | |
| SHP099 | ND | ND | ND | ND | ND | 7.11E−08 | 2.64E−06 |

These data demonstrate that ENT-03 (Compound III) is a potent inhibitor of PTP1B, and has potential therapeutic utility known to be associated with PTP1B inhibitors.

### Example 3: ENT-03 (Compound III) as a Weight Loss Agent in Mice

This example demonstrated the promotion of weight loss by ENT-03 (Compound III) in mice.

ENT-02 (MSI-1436) is known to induce weight loss through a mechanism that involves certain brain circuits that control appetite. Trodusquemine causes weight loss and a shift to lipid oxidation when administered systemically to mice. The pharmacological target appears to lie within the hypothalamus, including the arcuate nucleus, median eminence, and the paraventricular nucleus, based on localization of radioactive Trodusquemine, and cFos activation following intraventricular administration (Ahima et al., 2002).

Studies on the structure activity relationship of ENT-02, with respect to weight loss, have demonstrated the high degree of structural specificity required for this pharmacological effect (Zasloff et al., 2001). For example, altering the chirality of the spermine at C-3, the hydroxyl at C-7, or the methyl at C-21 eliminates weight loss. Activity is lost if the spermine is replaced by a spermidine, or if the terminal amino group of spermine is methylated. In contrast, both stereo-isomers of the sulfate at C-24 are equally active, demonstrating that spatial constraints are loosened around the anionic moiety, consistent with the activity observed for ENT-03.

To determine if ENT-03 (Compound III) exhibited a similar activity, male Swiss Webster mice, previously fed ad lib (about 50 grams, N=3 for each group), were treated i.p. with 5 doses of either ENT-02 (MSI-1436) or ENT-03 (Compound III) every other day at 10 mg/kg, e.g., on 20 days 0, 2, 4, 6, 8 and 10. Food was provided ad lib.

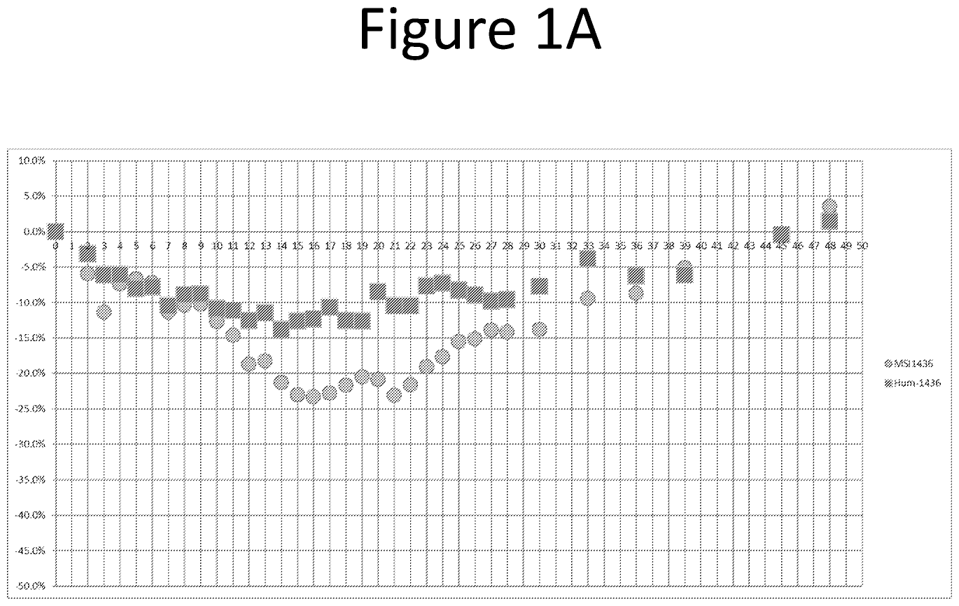

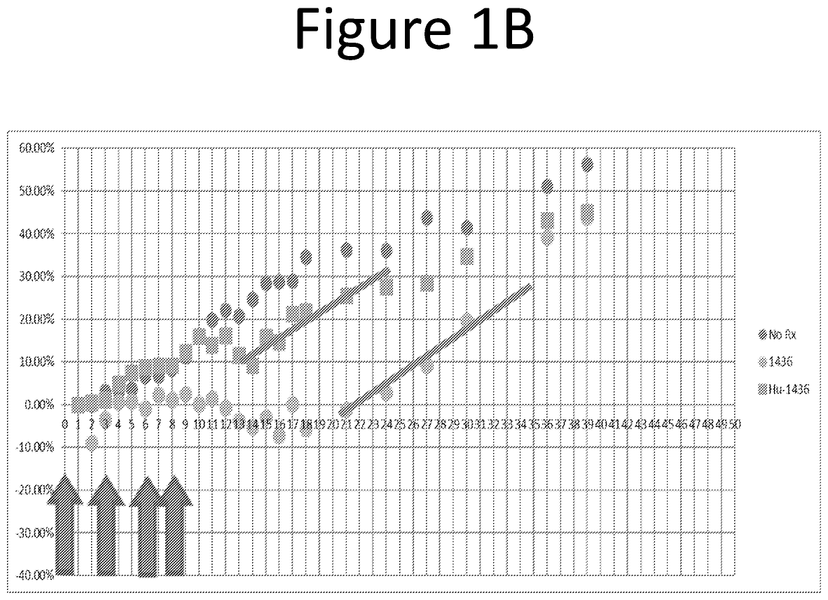

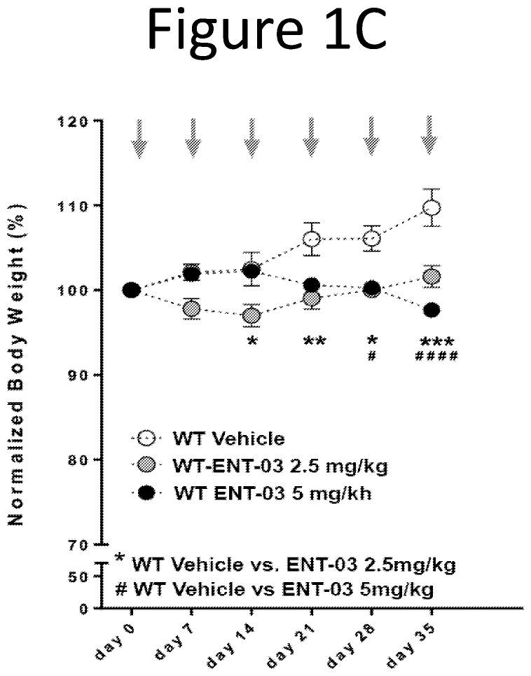

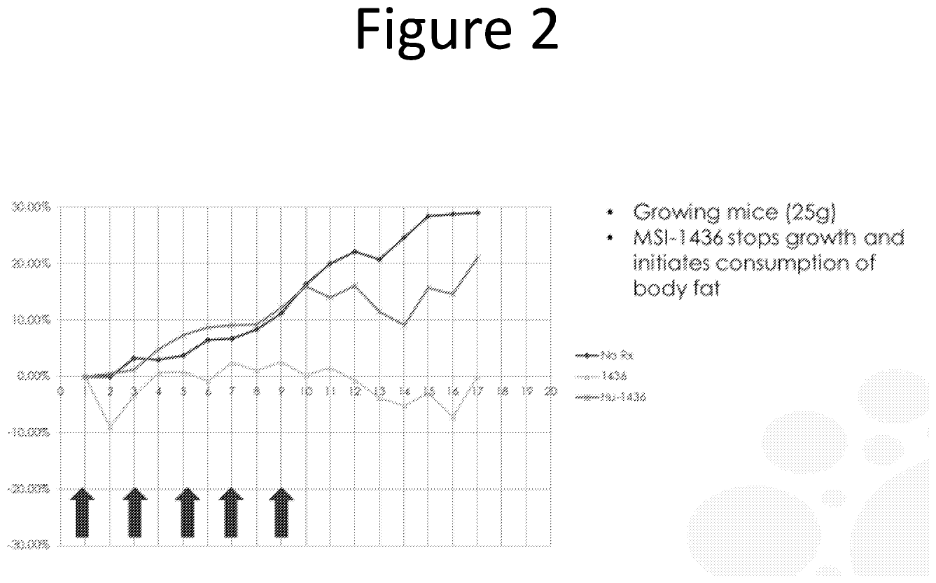

As can be seen in FIGS. 1A, 1B and 2, ENT-03 administration results in weight loss with kinetics similar to that seen following administration of ENT-02. Administration of both compounds resulted in a decrease in weight, with a nadir at about day 16, followed a gradual recovery to the starting weight by about day 45. While the initial decrease in weight was similar for both compounds, by about 10 days the effects diverged, with ENT-02 causing a more severe decline. By 27 days both sets of treated mice had lost about 10% of their starting body weight.

These data demonstrate that ENT-03 (Compound III) exhibits a pharmacological response with respect to weight similar to ENT-02 (MSI-1436) and suggests that ENT-03 (Compound III) can have utility in all therapeutic applications known to be associated with ENT-02 (MSI-1436) as well as other aminosterols.

In a second experiment, growing male mice, about 25 grams, were administered either compound following a similar dosing regimen. As in the previous experiment, both compounds affected weight gain. See FIG. 2. However, in the case of growing mice, ENT-02 had a more profound effect, having suppressed growth as well as having induced consumption of body fat. While the animals treated with ENT-03 continued normal growth, they “slimmed down”, suggesting that ENT-03 re-established a new optimal body weight “set point.”

In a third experiment, as seen in FIG. 1C intraperitoneal administration of ENT-03 to C57bl/6 male mice once weekly over 6 weeks caused a dose dependent weight loss. Other than weight loss the treated animals appeared clinically normal.

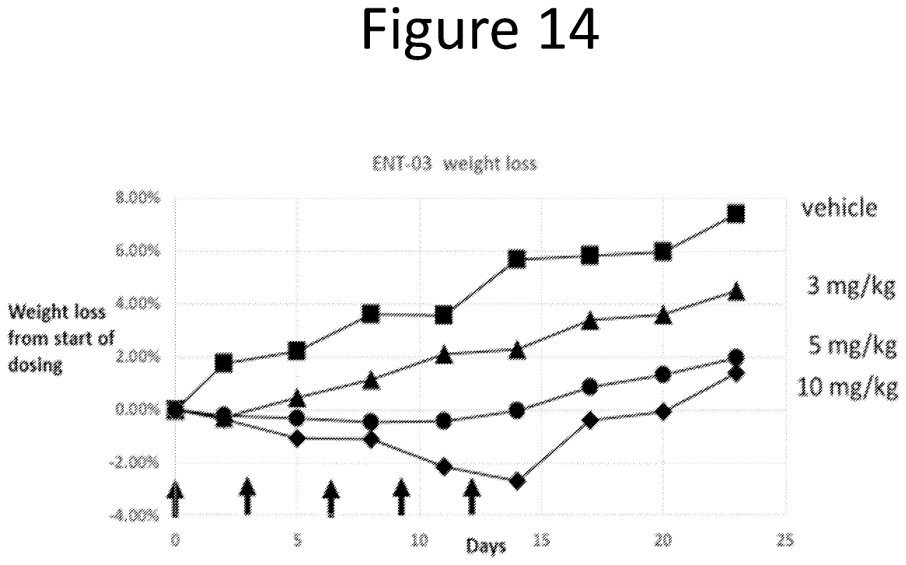

Finally, in a fourth experiment, as seen in FIG. 14, intraperitoneal administration of ENT-03 (3 mg/kg, 5 mg/kg, 10 mg/kg, or vehicle) to C57bl/6 male mice once weekly over 2 weeks caused a dose dependent weight loss. Other than weight loss the treated animals appeared clinically normal.

These studies demonstrate that ENT-03 (Compound III) exhibits potent pharmacological activity in mice, similar to ENT-02.

### Example 4: Rejuvenation of RNA Transcriptome in the Gut

The purpose of this example was to evaluate the impact of oral administration of ENT-02 and ENT-03 (Compound III) on young vs aged gut tissue.

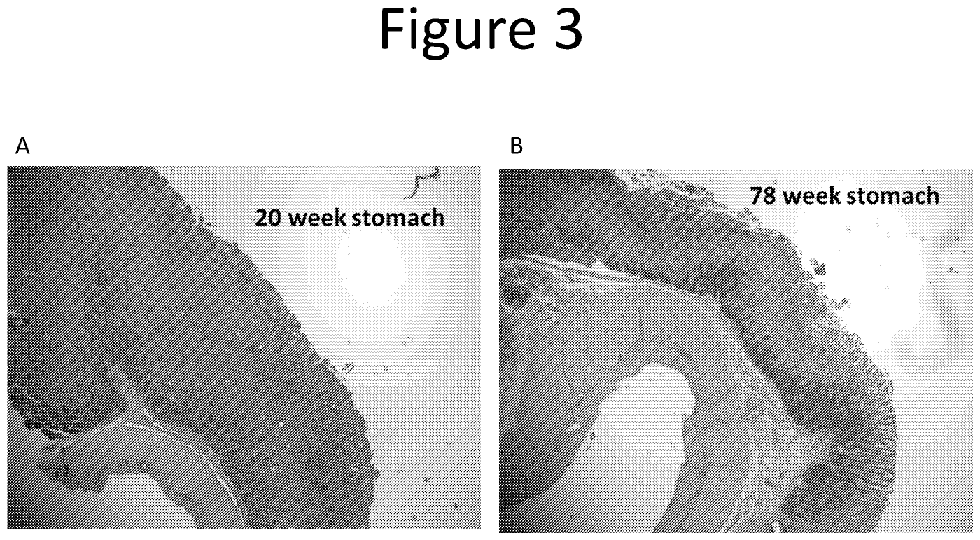

Aging involves a depletion of gene expression in the gut. Comparison of the images showing mucosal tissue in the stomach of a young mouse (20 week, FIG. 3A) versus an old mouse (78 week, FIG. 3B) shows a reduced thickness of the mucosal layer in the older specimen. This reduction in mucosa is associated with a reduced RNA transcriptome in the stomach in aged mice (78 weeks) vs. young mice (20 weeks), see Table 6 below. The present Example evaluated the impact of oral dosing of ENT-01 and ENT-02 and on old mice.

The dosing schedule used to determine the effect of orally administered squalamine and ENT-02 on the GI tracts of young and old mice was as follows. Male C57Bl/6 mice, aged 20 and 78 weeks, were obtained from Jackson labs. Animals were exposed to 12 hr light dark cycles and provided Teklad standard mouse diet and water ad lib. Animals were assigned to the treatment groups shown in Table 6.

TABLE 6

| | Dose | | | | Number |
|---|---|---|---|---|---|
| | Level* | | Dose | Age on | of |
| | (mg/kg/ | Concentration | Volume | Day 1 | Animals |
| Group | day) | (mg/ml) | (ml/kg) | (weeks) | Male |
| 1. Control | 0 | 0 | 10.0 | 20 | 5 |
| (vehicle) | | | | | |
| 2. ENT-01 | 40 | 4 | 10.0 | 20 | 5 |
| (Squalamine) | | | | | |
| 3. ENT-02 | 40 | 4 | 10.0 | 20 | 5 |
| (MSI-1436) | | | | | |
| 4. Control | 0 | 0 | 10.0 | 78 | 5 |
| (vehicle) | | | | | |
| 5. ENT-01 | 40 | 4 | 10.0 | 78 | 5 |
| (Squalamine) | | | | | |
| 6. ENT-02 | 40 | 4 | 10.0 | 78 | 5 |
| (MSI-1436) | | | | | |

Animals were dosed once daily by oral gavage in the morning for a total of 14 days. Animals were fasted 3-4 hours prior to dosing and at least 1 hour following dosing, with fasting not to exceed 6 hours. The test article was dissolved in 0.5% hydroxypropylcellulose in water. On day 15 the animals were euthanized by CO₂ asphyxiation, necropsied, and tissues prepared for histology and RNAseq analysis.

The GI tracts of the animals were sectioned into stomach, duodenum, jejunum, ileum, caecum, colon, and rectum. The tissues were then sent for histology, and the transcriptomes analyzed by RNAseq. Table 7 shows the respective mRNA amounts in young and old mouse stomach.

As shown in Table 8 Å below, mRNA levels for all of the genes in the table showed a significant increase after treatment with squalamine (ENT-01). Equally remarkable, while ENT-03 stimulates induction of the transcriptome of these segments of the GI tract of older mice, it has minimal effect in the younger animals, corresponding to a slight repression or induction of certain genes not associated with ageing. Perhaps ENT-03 is complementing a deficiency in the older mice that does not exist in the younger animals. This suggests that squalamine (ENT-01), and by extension structurally related aminosterols, such as ENT-03 (Compound III) and derivatives thereof, have a rejuvenating effect in the gut.

TABLE 7
Respective mRNA amounts in young and old mouse stomach

| | | | |
|---|---|---|---|
| Gene | Young | Old | P Value |

Epithelial Barrier Functions

| | | | |
|---|---|---|---|
| caspase_14 | 9.228 | 0.835 | 8.45E−10 |
| collagen_type_XVII_alpha_1 | 9.835 | 2.804 | 2.07E−04 |
| Corneodesmosin | 12.75 | 4.295 | 6.74E−04 |
| Cornifelin | 23.74 | 6.442 | 3.01E−05 |
| cystatin_E/M | 9.41 | 1.551 | 5.68E−07 |
| Dermokine | 117.5 | 23.86 | 1.34E−07 |
| desmocollin_1 | 16.09 | 4.175 | 2.6E−05 |
| desmoglein_1_beta | 9.592 | 2.625 | 1.41E−04 |
| Filaggrin | 21.67 | 2.207 | 3.81E−11 |
| gap_junction_protein_beta_4 | 1.882 | 0.239 | 2.52E−04 |
| gap_junction_protein_beta_6 | 2.125 | 0.298 | 2.11E−04 |
| H19_imprinted_maternally_expressed_transcript | 39.95 | 1.909 | 1.59E−17 |
| Hornerin | 13.6 | 3.519 | 3.23E−05 |
| kallikrein_related-peptidase_7_(chymotryptic_stratum_ | 10.14 | 0.656 | 1.33E−11 |
| keratin_1 | 332.6 | 69.79 | 1.72E−07 |
| keratin_10 | 337.7 | 45.93 | 8.03E−11 |
| keratinocyte_differentiation_associated_protein | 184.6 | 37.58 | 1.21E−07 |
| keratinocyte_expressed_proline-rich | 12.93 | 2.267 | 3.87E−07 |
| late_cornified_envelope_1A1 | 17.85 | 2.625 | 1.42E−08 |
| late_cornified_envelope_1A2 | 23.43 | 4.056 | 6.01E−08 |
| late_cornified_envelope_1B | 9.774 | 1.491 | 2.05E−07 |
| late_cornified_envelope_1C | 10.93 | 1.73 | 1.90E−07 |
| late_cornified_envelope_1E | 5.767 | 1.074 | 1.23E−05 |
| late_cornified_envelope_1F | 8.742 | 1.551 | 1.67E−06 |
| late_cornified_envelope_1G | 8.378 | 1.611 | 4.59E−06 |
| late_cornified_envelope_1H | 5.282 | 1.312 | 2.14E−04 |
| late_cornified_envelope_1I | 8.682 | 1.074 | 3.37E−08 |
| late_cornified_envelope_1J | 5.16 | 0.895 | 1.01E−05 |
| late_cornified_envelope_1L | 4.553 | 0.596 | 1.82E−06 |
| late_cornified_envelope_1M | 16.76 | 1.133 | 5.22E−13 |
| late_cornified_envelope_3C | 5.525 | 1.491 | 3.92E−04 |
| late_cornified_envelope_3E | 5.889 | 1.491 | 3.92E−04 |
| late_cornified_envelope_3F | 10.81 | 3.281 | 2.66E−04 |
| lectin_galactose_binding_soluble_7 | 139.3 | 25.77 | 2.70E−08 |
| Loricrin | 275.3 | 45.75 | 3.37E−09 |
| Sciellin | 11.11 | 3.221 | 1.56E−04 |

Muscle Tissue

| | | | |
|---|---|---|---|
| Myoglobin | 47.9 | 2.923 | 4.10E−16 |
| myosin_binding_protein_C_slow-type | 15.06 | 1.133 | 4.01E−12 |
| myosin_heavy_polypeptide_1_skeletal_muscle | 73.46 | 2.982 | 3.47E−20 |
| myosin_heavy_polypeptide_8_skeletal_muscle | 90.46 | 3.34 | 2.67E−21 |
| myosin_light_chain_phosphorylatable_fast_ske | 30.23 | 4.235 | 1.42E−09 |
| myosin_light_polypeptide_3 | 26.77 | 1.312 | 2.72E−16 |
| myozenin_1 | 6.617 | 0.418 | 3.87E−10 |
| myozenin_2 | 4.31 | 0.239 | 7.95E−09 |
| titin-cap | 17.36 | 0.418 | 3.66E−18 |

TABLE 8A
respective mRNA amounts in old mice versus old mice treated with
squalamine (ENT-01)

| | | | |
|---|---|---|---|
| Gene | Old | Old + Ent-01 | P Value |

Epithelial Barrier Functions

| | | | |
|---|---|---|---|
| caspase_14 | 0.795 | 6.077 | 2.25E−07 |
| collagen_type_XVII_alpha_1 | 2.668 | 9.509 | 1.43E−04 |
| Corneodesmosin | 4.087 | 7.933 | 4.10E−02 |
| Cornifelin | 6.131 | 22.17 | 4.07E−05 |
| cystatin_E/M | 1.476 | 8.721 | 6.89E−07 |
| Dermokine | 22.71 | 88.45 | 5.65E−06 |
| desmocollin_1 | 3.974 | 8.102 | 2.83E−02 |
| desmoglein_1_beta | 2.498 | 7.089 | 2.14E−03 |
| Filaggrin | 2.1 | 16.32 | 2.05E−09 |
| gap_junction_protein_beta_4 | 0.227 | 1.294 | 3.15E−03 |
| gap_junction_protein_beta_6 | 0.284 | 1.575 | 1.59E−03 |
| H19_imprinted_maternally_ | 1.817 | 13.73 | 5.79E−09 |
| expressed_transcript | | | |
| Hornerin | 3.349 | 11.82 | 1.09E−04 |
| kallikrein_related-peptidase_7_ | 0.624 | 5.908 | 3.74E−08 |
| (chymotryptic_stratum | | | |
| keratin_1 | 66.42 | 348.9 | 3.28E−08 |
| keratin_10 | 43.71 | 310.3 | 1.56E−10 |
| keratinocyte_differentiation_ | 35.77 | 181.2 | 7.27E−08 |
| associated_protein | | | |
| keratinocyte_expressed_proline-rich | 2.157 | 7.99 | 1.36E−04 |
| late_cornified_envelope_1A1 | 2.498 | 10.97 | 1.03E−05 |
| late_cornified_envelope_1A2 | 3.86 | 17.78 | 2.08E−06 |
| late_cornified_envelope_1B | 1.419 | 8.102 | 1.33E−06 |
| late_cornified_envelope_1C | 1.646 | 7.202 | 3.26E−05 |
| late_cornified_envelope_1E | 1.022 | 4.501 | 1.17E−04 |
| late_cornified_envelope_1F | 1.476 | 6.47 | 3.92E−05 |
| late_cornified_envelope_1G | 1.533 | 4.557 | 2.78E−03 |
| late_cornified_envelope_1H | 1.249 | 4.164 | 1.38E−03 |
| late_cornified_envelope_1I | 1.022 | 7.708 | 8.29E−08 |
| late_cornified_envelope_1J | 0.852 | 3.376 | 6.62E−04 |
| late_cornified_envelope_1L | 0.568 | 2.644 | 5.13E−04 |
| late_cornified_envelope_1M | 1.079 | 8.158 | 6.04E−08 |
| late_cornified_envelope_3C | 1.419 | 3.263 | 2.79E−02 |
| late_cornified_envelope_3E | 1.419 | 4.332 | 2.50E−03 |
| late_cornified_envelope_3F | 3.122 | 9.115 | 1.15E−03 |
| lectin_galactose_binding_soluble_7 | 24.52 | 142.5 | 7.67E−09 |
| Loricrin | 43.54 | 209.5 | 1.63E−07 |
| Sciellin | 3.066 | 9.79 | 4.25E−04 |

Muscle Tissue

| | | | |
|---|---|---|---|
| Myoglobin | 2.782 | 17.72 | 2.49E−08 |
| myosin_binding_protein_C_slow-type | 1.079 | 3.173 | 1.42E−03 |
| myosin_heavy_polypeptide_1_skeletal_ | 2.838 | 11.03 | 4.18E−05 |
| muscle | | | |
| myosin_heavy_polypeptide_8_skeletal_ | 3.179 | 14.97 | 2.20E−06 |
| muscle | | | |
| myosin_light_chain_phosphorylatable_ | 4.031 | 15.92 | 1.79E−05 |
| fast_ske | | | |
| myosin_light_polypeptide_3 | 1.249 | 10.58 | 4.49E−09 |
| myozenin_1 | 0.397 | 2.476 | 1.19E−04 |
| myozenin_2 | 0.227 | 1.519 | 8.72E−04 |
| titin-cap | 0.397 | 3.376 | 3.15E−06 |

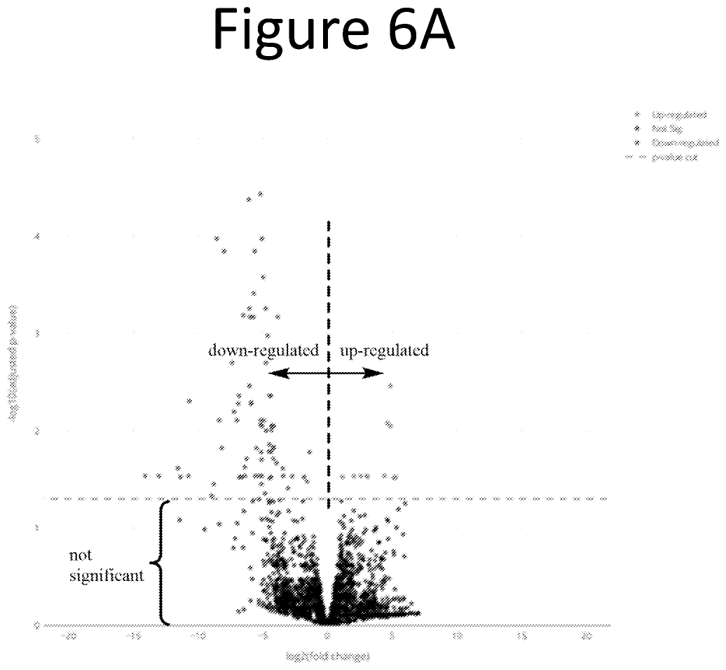

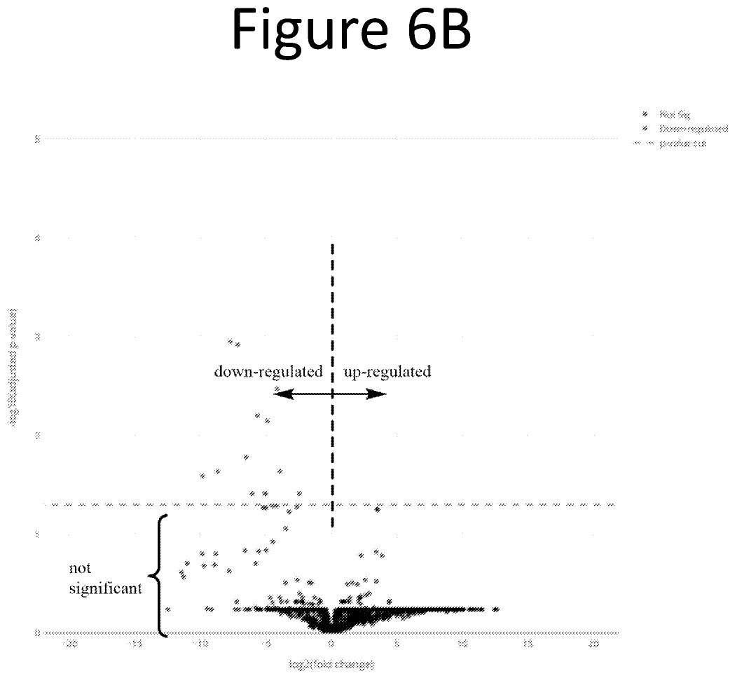

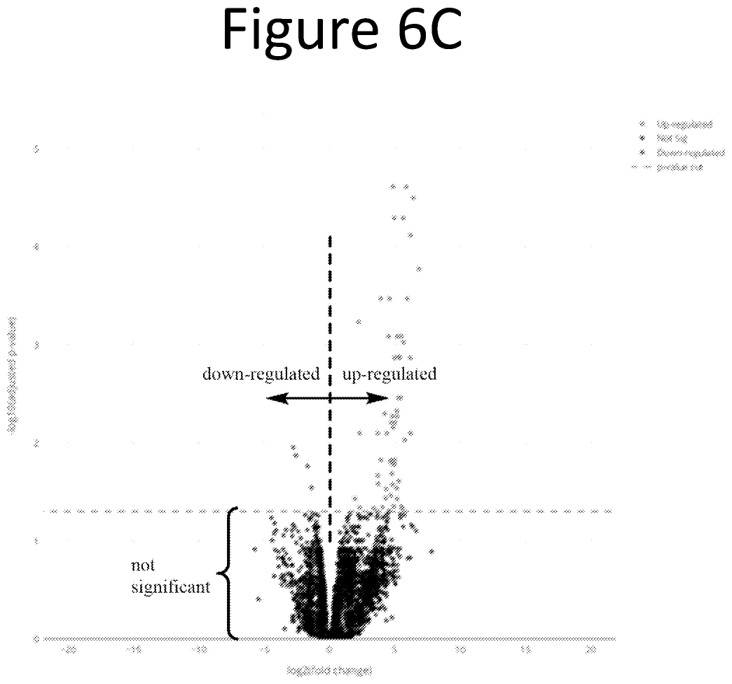

Applicant also investigated transcriptome changes within each segment of the GI tract. Upon oral administration to aged mice the abundance of gene transcripts that decrease with ageing in the stomach, jejunum, and ileum are increased, in some cases to levels observed in the younger individuals as shown in FIGS. 6A-6C.

Young (20 week) and aged (80 week) C57bl/6 male mice were administered ENT-03 (40 mg/kg) or vehicle (water) by oral gavage daily for 2 weeks. Over this period of time clinical observations were unremarkable as were the gross and microscopic examinations of the GI tract. The transcriptomes of the stomach, jejunum and ileum were analyzed by RNAseq technology. Comparative analyses of the transcripts from untreated animals identified numerous transcripts that differed in abundance between the corresponding tissues from old and young individuals. Differential expression was observed for 86 stomach transcripts (p(adj)<0.05): 70 decreased, and 16 increased with ageing; in the case of the jejunum, over 400 transcripts decreased, 200 increased with ageing; and for the ileum, 700 transcripts decreased, while 400 increased with ageing. Thus, within these three distinct regions of the mouse GI tract, ageing is associated with changes in the transcriptome.

When the effect of oral administration of ENT-03 on the transcriptome of the tissues was examined, in each case it was observed that the transcriptional response in the young was blunted in comparison to that occurring in the aged animals (FIGS. 6A-6C). For example, in the stomach of treated young animals, 13 genes were differentially expressed (p(adj)<0.05), while 63 were in the aged group. Similarly, for the jejunum 42 genes were differentially expressed in the young, and 382 genes on treatment in the aged group. In the ileum, 80 genes were differentially expressed in the young, and 1162 genes in the aged animals treated with ENT-03.

Next, the sets of genes that were differentially expressed (DEGs) with ageing were compared with those that were differentially expressed in the treated aged animals or in the treated young animals. Analysis of the overlapping DEGs between contrasts revealed, for example, in the stomach 37 genes down-regulated in ageing (old versus young) significantly overlapped (P<0.0001, hypergeometric test) with genes up-regulated by treatment with ENT-03 in old mice. In contrast the only significant overlap between the stomach ageing genes and those in the young ENT-03 treated stomach were 12 genes that were further down regulated in the ageing direction.

The “ageing” genes that ENT-03 appears to complement most significantly within the stomach are listed in Table 8B. Notably, in the stomach, the “restored” ageing genes include those involved in tissue renewal (fibroblast growth factor 2; zinc finger protein 383; forkhead box C2); neuronal differentiation (neural cell adhesion molecule 2); immunity (toll-like receptors 9 and 12; interleukin 2 receptor, beta chain), neurotransmitter synthesis and uptake (choline and serotonin transporters), and mitochondrial respiration (cytochrome c oxidase subunit 6B2).

TABLE_8B
Ageing genes complemented by ENT-03 (30 most significant) Stomach
zinc finger protein 382
nuclear receptor 2C2-associated protein
CD300 molecule like family member d
fibroblast growth factor 2
transmembrane protein 71
zinc finger protein 449
guanine nucleotide binding protein (G protein), gamma 8
synaptophysin-like 2
interleukin 2 receptor, beta chain
nuclear assembly factor 1 ribonucleoprotein
predicted gene 20604
toll-like receptor 12
predicted gene 14322
forkhead box C2
solute carrier family 5 (choline transporter), member 7
Ras association and DIL domains
cytochrome c oxidase subunit 6B2
neural cell adhesion molecule 2
solute carrier family 6 (neurotransmitter transporter, serotonin), member 4
ELAV like RNA binding protein 3
shadow of prion protein
sortilin-related VPS10 domain containing receptor 1
FXYD domain-containing ion transport regulator 2
toll-like receptor 9
claudin 34C1

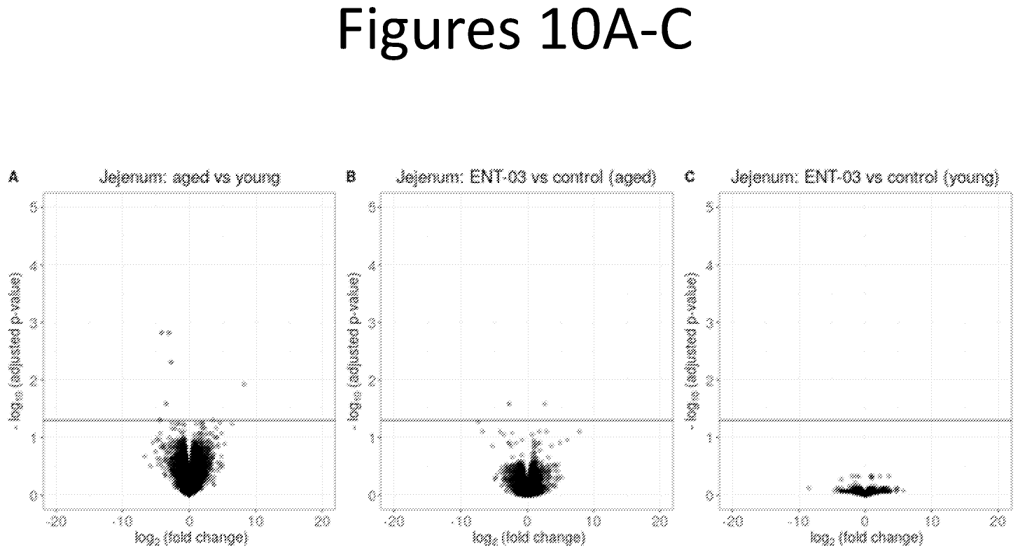

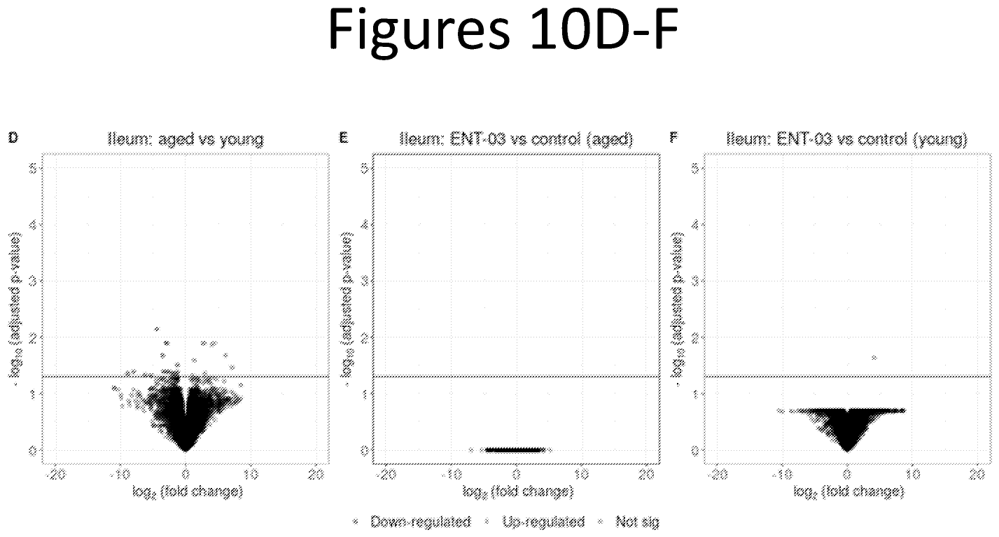

First, genes that significantly changed in expression between young and aged mice were identified (FDR-adjusted p-value <0.05) (FIG. 6A). With ageing, the expression of 75 genes in the stomach was significantly decreased, and the expression of 11 genes was significantly increased (FIG. 6A). Meanwhile, fewer differences were observed between the gene expression profiles of the jejunum (FIGS. 10A-10D) or ileum (FIGS. 10D-10F) of young and aged mice. In the jejunum, 5 genes decreased in expression and 2 genes increased in expression with ageing. Whereas in the ileum, 19 genes decreased and 9 genes increased in expression. These results are consistent with the recognized reduced regenerative capacity of the ageing rodent stomach (Fukunaga et al., 1998), and the resilience with ageing of the small intestine (Eswaran et al., 2006).

Following oral administration of ENT-03 to young mice (20 weeks), 13 stomach genes responded and all were transcriptionally repressed by ENT-03 treatment (FIG. 6B). The expression of no more than 2 genes changed significantly in response to ENT-03 exposure in the jejunum and ileum for both young and aged mice.

In contrast, a more robust effect on gene expression in the stomach was observed in the older animals (78 weeks) treated with ENT-03. 63 genes were transcriptionally induced (FDR-adjusted p<0.05) (FIG. 6C). Interestingly, 36 of the genes that increased in expression upon ENT-03 treatment of aged mice were the same that decreased in expression with ageing. These genes include those involved in tissue renewal (fibroblast growth factor 2 (Fgf2), zinc finger protein 382 (Zfp382) and forkhead box C2 (Foxc2)); in neuronal differentiation (neural cell adhesion molecule 2 (Ncam2)); in immunity (toll-like receptors 9 and 12 (Tlr9, Tlr12), interleukin 2 receptor, beta chain (Il2rb) and CD300 (Cd300ld)); in neurotransmitter synthesis and uptake (choline and serotonin transporters (Slc5a7, Slc6a4)); and in mitochondrial respiration (cytochrome c oxidase subunit 6B2 (Cox6b2)).

Statistical analysis of RNA-sequencing data was performed using R programming language. Transcripts with less than one read count per million reads in all samples of each tissue were removed. The raw count data for the samples were then normalised using trimmed mean of M-values normalisation and transformed with voom (Law et al., 2014), resulting in log 2-transformed counts per million with associated precision weights. Normalised data provide the input for statistical hypothesis testing, in which genes that are significantly different between sample groups are identified. Statistical comparisons were performed using linear modelling, as implemented in the Bioconductor package limma (Ritchie et al., 2015). Significance values (p-values) were adjusted for multiple testing using Benjamini-Hochberg procedure (Benjamini et al., 1995). For each comparison (e.g. group A versus group B), a positive log 2-transformed fold change indicates up-regulation in group A relative to group B.

To investigate the overlap between contrasts, the number of overlapping differentially expressed genes (defined using adjusted p-value <0.05) between all pairwise combinations of the comparisons performed were counted. For each comparison, the Jaccard index, defined as the intersection over the union, measures the similarity between the two contrasts under consideration. The p-value was calculated using a hypergeometric test taking into account the universe size under the assumption that the contrasts and genes are independent.

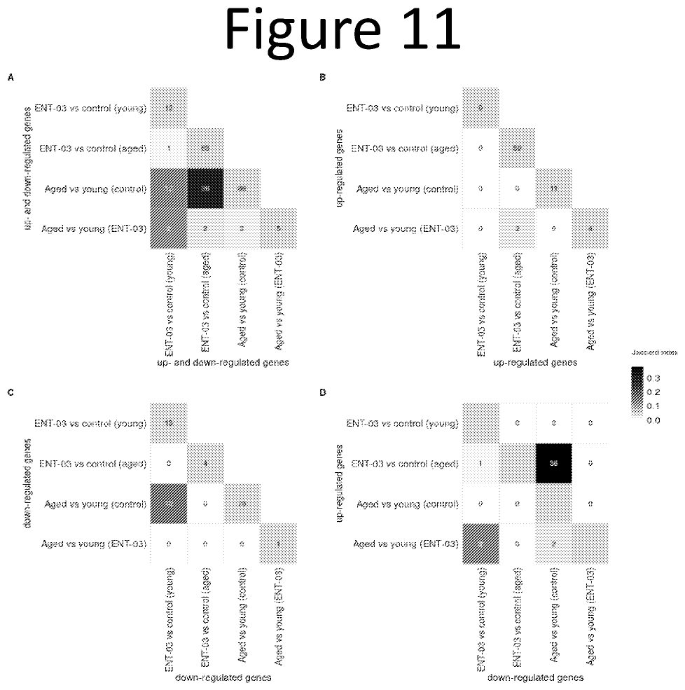

FIG. 11 shows a set of heatmaps investigating the overlap of differentially expressed genes between pairs of contrasts.

These data support the hypothesis that oral administration of ENT-03 to old mice can reverse some of the changes in gene expression associated with ageing within the GI tract.

### Example 5: Pharmacokinetic Study of ENT-03 (Compound III) Via Intravenous and Oral Administration

The objective of this example was to determine the pharmacokinetic profile of ENT-03 (Compound III) via intravenous and oral administration in male SD Rats.

The study groups are shown in Table 9 below.

TABLE 9
Study Groups

| | | Dose | Dose | | Admin- | |
|---|---|---|---|---|---|---|
| | | Level* | Volume | Conc. | istration | No. of |
| Group | Treatment | (mg/kg) | (mL/kg) | (mg/ml) | Route | Animals |
| 1 | ENT-03 | 5 | 5 | 1 | IV | 3 male |
| 2 | (Compound III) | 5 | 10 | 0.5 | PO | 3 male |

*The salt factor of 1.177 is used for the formulation preparation of Compound III.

Animal feeding control: Animals were food fasted overnight prior to dosing and fed 4 hours after administration having free access to water.

Dose formulation processing during dosing: The dose formulation was kept stirring at room temperature and was used within 2 hours.

TABLE 10
PK Time Points

| Group | PK time points |
|---|---|
| 1 | Plasma: 5 min, 15 min, 30 min, 1, 2, 4, 8, 24 hr post dose |
| 2 | Plasma: 15 min, 30 min, 1, 2, 4, 8, 24 hr post dose |

TABLE 11
Acceptable Time Ranges for Blood Collection

| Time Points | | Acceptable Time Ranges | |
|---|---|---|---|
| 5-15 | min | Within | ± 0.25 minute |
| 0.5-2 | hr | Within | ± 2 minutes |
| 4-24 | hrs | Within | ± 10 minutes |

TABLE 12
Blood Sample Collection and Processing

| Collection Site: | Jugular vein |
|---|---|
| Volume Collected: | ~0.2 mL per time point |
| Anticoagulant: | K2EDTA anti-coagulant tube |
| Plasma Samples | 1) Approximately 0.2 mL blood was collected |
| Processing and | at each time point. Blood of each sample |
| Storage: | was transferred into plastic micro centrifuge |
| | tubes containing anticoagulant of K2EDTA. |
| | 2) Blood samples were centrifuged at 4,000 g |
| | for 5 minutes at 4° C. to obtain plasma. |
| | 3) The samples were stored in a freezer at |
| | −75 ± 15° C. prior to analysis. |

TABLE 13
Evaluations during the In-life Phase

| Procedure | Frequency |
|---|---|
| Cageside Observations: | Twice daily |
| Detailed Clinical Observations: | Once prior to dosing on Day 1 |
| Body Weight: | Once prior to each dosing |

Note:
Unscheduled clinical observations (cageside or detailed) were also performed, as needed.

TABLE 14
Test System

| | |
|---|---|
| Species/Strain/Sex: | Rat/SD/Male |
| Source: | Si Bei Fu Laboratory Animal |
| | Technology Co. Ltd |
| Age at Day of Dosing: | Approximately 6-8 weeks |
| Weight at Day of Dosing: | Approximately 200-300 ga |

TABLE 15
Compound Identification

| Compound Identification | MW | FW | Salt Factor |
|---|---|---|---|
| ENT-03 (Compound III) | 618.98 | 728.36 | 1.177 |

TABLE 16
Dose Formulation

| | |
|---|---|
| Formulation | Freshly prepared on the day of dosing |
| Frequency: | |
| Vehicle Composition: | IV&PO: Sterile water |
| Storage Condition: | Dose formulation for dosing: Room temperature |
| Preparation | Weigh out test articles and dissolve it into |
| Procedures: | vehicle with sonication, vortexing to obtain |
| | the targeted concentration of test articles and |
| | vehicle component. |
| Dosing Site | For IV dosing, the animals were administered |
| | intravenously via tail vein. |
| | For PO dosing, the animals were administered |
| | via oral gavage. |

PK Sample Analyses: Concentrations of ENT-03 (Compound III) in the plasma and dose samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data.

IV Bolus administration: T_{1/2}, C₀, AUCₗₐₛₜ, AUC_{inf}, MRT_{inf}, Cl, Vss, Number of Points for Regression.

PO administration: F, T_{1/2}, Cₘₐₓ, Tₘₐₓ, AUC_{inf}, AUCₗₐₛₜ, MRT_{inf}, Number of Points for Regression.

The pharmacokinetic data was described using descriptive statistics such as mean and standard deviation. All the residual biological samples were retained in the freezer (˜75±15° C.) for a period of 6 months after testing. The results are tabulated below in Table 17.

TABLE 17
Summary of ENT-03 (Compound III) I.V. (group 1) plasma PK parameters

| PK parameters | Unit | Rat 1 | Rat 2 | Rat 3 | Mean | SD | CV(%) |
|---|---|---|---|---|---|---|---|
| Cl_obs | mL/min/kg | 0.0903 | 0.0885 | 0.0761 | 0.0850 | 0.0078 | 9.16 |
| T_{1/2} | h | 18.6 | 17.5 | 18.6 | 18.2 | 0.6 | 3.55 |
| C₀ | ng/ml | 80402 | 102409 | 76200 | 86337 | 14076 | 16.3 |
| AUCₗₐₛₜ | h * ng/ml | 557542 | 608321 | 661154 | 609005 | 51810 | 8.51 |
| AUC_{Inf} | h * ng/ml | 922477 | 941130 | 1095763 | 986457 | 95121 | 9.64 |
| AUC__{%Extrap}_obs | % | 39.6 | 35.4 | 39.7 | 38.2 | 2.5 | 6.42 |
| MRT_{Inf}_obs | h | 25.3 | 22.9 | 25.5 | 24.6 | 1.5 | 5.96 |
| AUCₗₐₛₜ/D | h * mg/mL | 111508 | 121664 | 132231 | 121801 | 10362 | 8.51 |
| Vₛₛ_obs | L/kg | 0.137 | 0.122 | 0.116 | 0.125 | 0.011 | 8.55 |

### Example 6: ENT-03 (Compound III) Reverses Alzheimer's in Mice

PTP1B dependent mechanisms have been utilized for reversal of memory impairment and normalization of behavior and reduction in neuronal loss in beta amyloid and tau mouse models of Alzheimer's disease (Ricke, Cruz et al. 2020). Other studies have shown reduction in the toxicity of beta amyloid aggregates by trodusquemine in vitro and in a C. elegans model of Alzheimer's disease (Limbocker, Chia et al. 2019).

ENT-02 (MSI-1436) reverses several conditions (in mice) that are associated with ageing, such as metabolic syndrome, Alzheimer's disease, atherosclerosis, cancer and a reduced capacity for regenerative repair. As we have shown in this report, ENT-03 can treat Alzheimer's disease in murine models.

The Morris water maze was used to test the effect of ENT-03 on spatial learning and memory deficits in 2 mouse models of familial Alzheimer's disease, hAPP-J20 mice that express a double mutant of the human amyloid precursor protein (Mucke et al., 2000), and PS19 mice that express the P301S mutant of the human microtubule associated protein tau (Yoshiyama et al., 2007).

hAPP-J20 mice expressing human APP bearing the Swedish and Indiana familial mutations (B6.Cg-Zbtb20Tg(PDGFB-APPSwInd)20Lms/2Mmjax, (Mucke et al., 2000)) and PS19 mice expressing the P301S mutation of human tau protein (B6;C3-Tg(Prnp-MAPT*P301S)PS19Vle/J (Yoshiyama et al., 2007)) were purchased from Jackson laboratories and maintained as colonies in the animal facility of the University of Ottawa. Heterozygous hAPP-J20 and PS19 mice were used. Genotypes were verified by PCR using genomic DNA isolated from tail or ear biopsies.

Clinical grade ENT-03 (provided by Enterin, Inc.) was administered intraperitoneally (i.p.) once per week for 6 weeks at a dose of 2.5 mg/kg bodyweight starting at 4.5 months of age. The behavioral experiments were conducted 10 days after the last injection. Vehicle treated controls received sterile saline (0.9% NaCl in water).

The Morris water maze test to analyze the ability of mice to learn and remember the location of a submerged platform in a pool containing opaque water was conducted in the Faculty of Medicine Behavior Core Laboratory at the University of Ottawa. The 80 cm² platform surface corresponds to 0.6% of the total pool area. Mice were habituated to the experimental room and consecutive experiments were performed between noon and 4 PM. Mice were trained for 5 days, (four trials per day with an inter-trial interval of 20 minutes and a random start location in one of four positions) to find the invisible, submerged platform at a fixed location. Cues around the pool were provided as spatial references. Trials lasted 1 minute or until the mouse found the platform. Mice were guided to the platform if they did not find the platform. Mice stayed on the platform for 15 seconds of each trial before being removed to their cages. After the training period, the platform was removed from the pool and the probe trial was executed within 1 minute on the following day. On the probe day, crossings of the platform area and target quadrant were counted and swimming speed was measured using Ethovision automated video tracking software (Noldus).

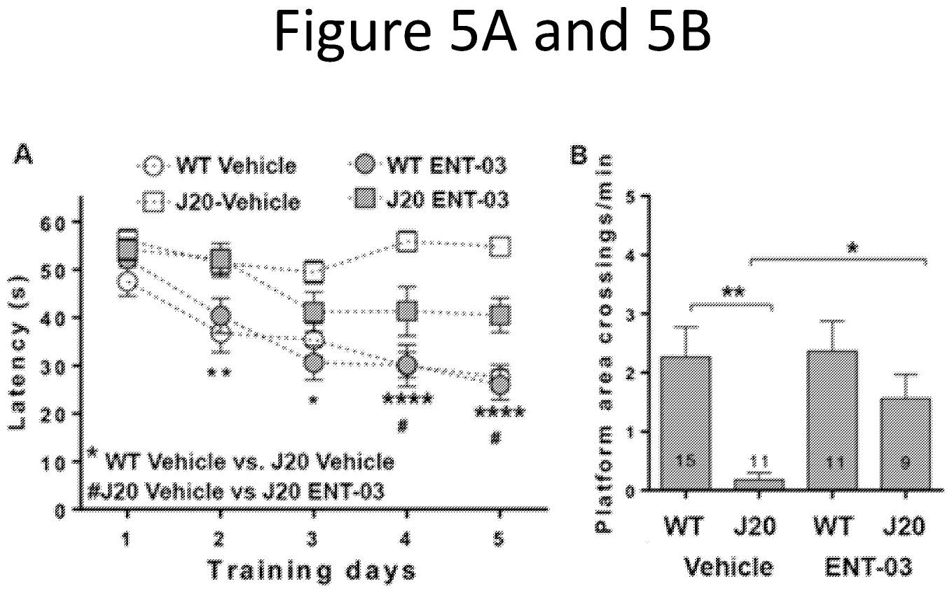

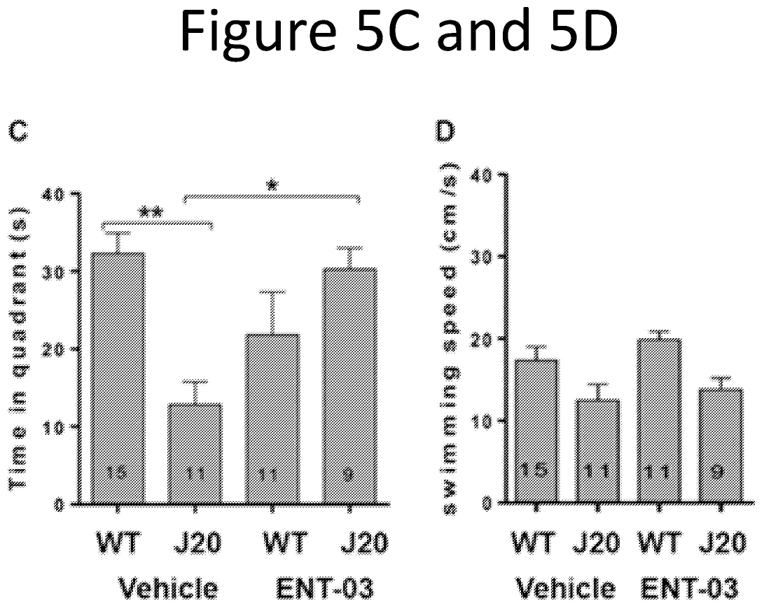

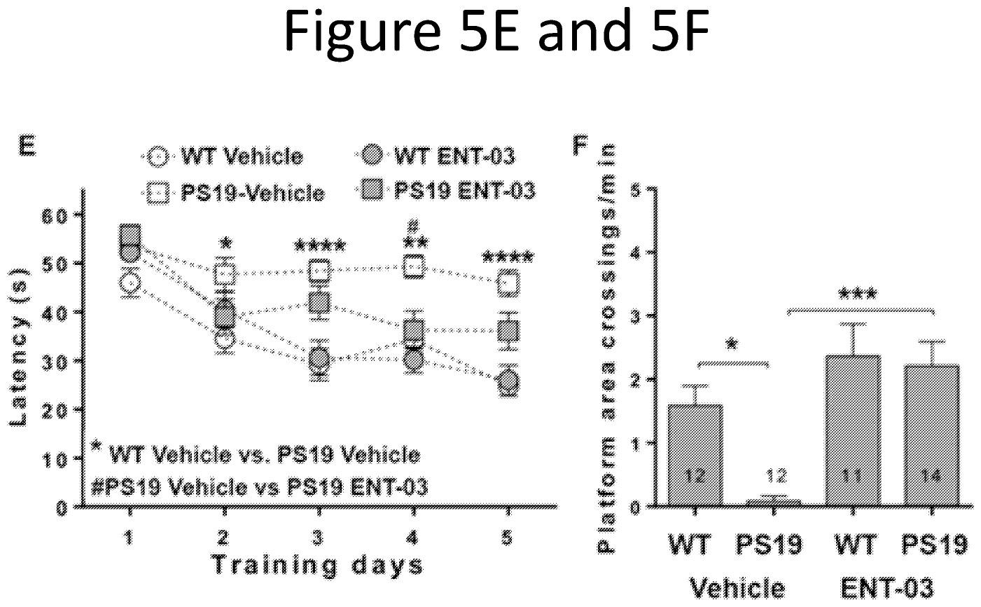

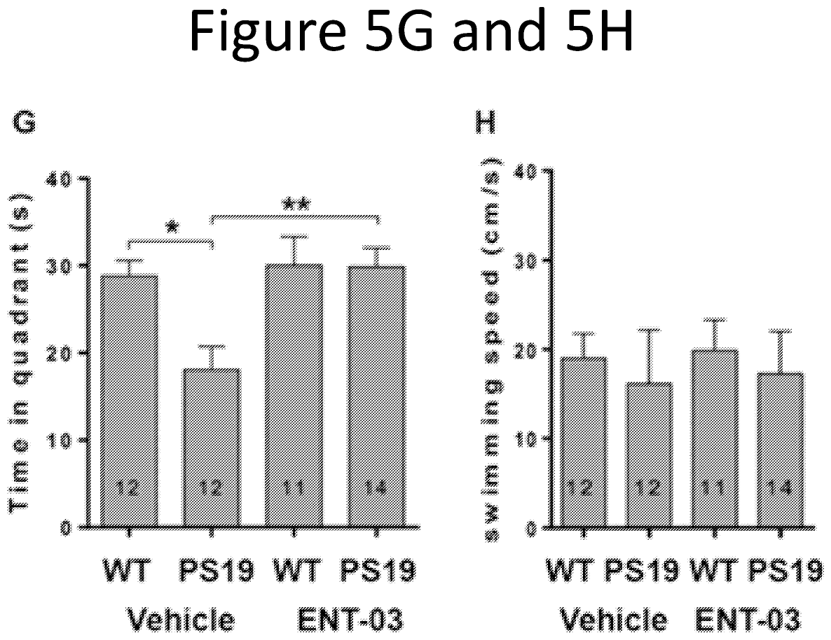

In both disease models, one with amyloidopathy and the other with a tauopathy, learning during the training phase (FIGS. 5A and 5E) and memory on the probe day (FIGS. 5B, 5C, 5F and 5G) were improved with ENT-03 treatment compared to vehicle treated hAPP-J20 or PS19 littermate control mice (FIG. 5). The effect on preventing cognitive decline is similar to what has been observed with the related compound trodusquemine hAPP-J20 and PS19 mice (Ricke et al., 2020).

In particular, Morris water maze test reveals ENT-03 treatment over 6 weeks (2.5 mg/kg/wk, i.p.) decreased the latency to find the hidden platform for hAPP-J20 mice during the training phase (FIGS. 5A and 5E) and improved memory (crossings at the area where the platform was located (FIGS. 5B and 5F) and time in the platform area (FIGS. 5C and 5G) on the probe day. (FIGS. 5D and 5H) Swimming speed. FIG. 5A, repeated measures two-way ANOVA: genotype/treatment, F(3,42)=13.47, p<0.0001; time, F(4,168)=23.45, p<0.0001; subjects (matching), F(42,168)=4.420, p<0.0001; interaction, F(12,168)=3.458, p=0.0001; *, post hoc pairwise comparisons between Veh WT and Veh hAPP-J20, day 2, 3, 4, 5: p=0.0073, 0.0101, <0.0001, <0.0001, respectively; #, post hoc pairwise comparisons between Veh hAPP-J20 and ENT-03 hAPP-J20, day 4, 5: p=0.0260, 0.0298, respectively. FIG. 5B, two-way ANOVA: genotype, F(1,42)=10.44, p=0.0024; treatment, F(1,42)=4.509, p=0.0396; interaction, F(1,42)=3.625, p=0.0638; *, post hoc comparisons between Veh WT and Veh hAPP-J20, p=0.0020 and between Veh and ENT-03 hAPP-J20, p=0.0487. FIG. 5C, two-way ANOVA: genotype, F(1,41)=9.977, p=0.0030; treatment, F(1,41)=4.149, p=0.0482; interaction, F(1,41)=1.570, p=0.2173). *, post hoc comparisons between Veh WT and Veh hAPP-J20, p=0.0080 and between Veh hAPP-J20 and ENT-03 hAPP-J20, p=0.0032. FIG. 5D, two-way ANOVA, genotype, F(1,42)=10.95, p=0.0019; treatment, F(1, 42)=1.352, p=0.2515; interaction, F(1,42)=0.1378, p=0.7123. FIG. 5E, repeated measures two-way ANOVA: genotype/treatment, F(3,45)=11.90, p<0.0001; time, F(4,180)=29.09, p<0.0001; subjects (matching), F(45,180)=2.780, p<0.0001; interaction, F(12,180)=2.325, p=0.0087; *, post hoc pairwise comparisons between Veh WT and Veh hAPP-J20, day 2, 3, 4, 5: p=0.0143, <0.0001, 0.0037, <0.0001, respectively; #, post hoc pairwise comparisons between Veh hAPP-J20 and ENT-03 hAPP-J20, day 4: p=0.0111. FIG. 5F, two-way ANOVA: genotype, F(1,45)=5.413, p=0.0245; treatment, F(1,45)=16.87, p=0.0002; interaction, F(1,45)=3.630, p=0.0631; *, post hoc comparisons between Veh WT and Veh hAPP-J20, p=0.0236 and between Veh hAPP-J20 and ENT-03 hAPP-J20, p=0.0004. FIG. 5G, two-way ANOVA: genotype, F(1,45)=4.829, p=0.0332; treatment, F(1,45)=6,861, p=0.0120; interaction, F(1,45)=4.500, p=0.0394). *, post hoc comparisons between Veh WT and Veh hAPP-J20, p=0.0201 and between Veh hAPP-J20 and ENT-03 hAPP-J20, p=0.0064. FIG. 5H, two-way ANOVA, genotype, F(1,45)=4.572, p=0380; treatment, F(1, 45)=0.6015, p=0.4421; interaction, F(1,45)=0.0090, p=0.9249).

### Example 7: Detection of ENT-03 in Humans and Mouse Pup Brain

In 1992 Nagata et al identified high concentrations of the bile acid, 7-alpha hydroxy-3-oxo-4-cholestenoic acid (7-HOCA), in human chronic subdural hematoma fluid, (FIG. 1C) (Nagata et al., 1992) and later in acute subarachnoid hemorrhage (Nagata et al., 1995). In 1997, Zhang et al reported that rat brain cells could metabolize 27-hydroxycholesterol to 7-HOCA (Zhang et al., 1997). Subsequently, Bjorkhem, Sjövall, Griffiths and their collaborators identified 7-HOCA as the most abundant bile acid in human cerebrospinal fluid (Ogundare et al., 2010; Meaney et al., 2007; Saeed et al., 2014; Saeed et al., 2014). 7-HOCA did not appear to a be a biologically active bile acid, in that it did not activate either the FXR, LXR, or RXR/NURR1 receptors, for which bile acids and oxysterols are known ligands (Ogundare et al., 2010). Björkhem has proposed that the brain metabolizes 27-hydroxycholesterol that enters from the periphery into 7-HOCA to facilitate the efflux of the oxysterol back into the circulation (Meaney et al., 2007).

Applicant suggests that the presence of ENT-03 in subdural hematoma fluid reflects its possible role in the development of this structure. Following head injury, generally in older people and rarely in infants, a highly vascularized sac-like “organ” develops, with one side deriving from the dura(“outer membrane”), the other from the subarachnoid (“inner membrane”) (Yamashima et al., 2000). High concentrations of numerous growth factors, including VEGF accumulate within the fluid (Edlmann et al., 2017). We suggest that ENT-03 appears within the subdural hematoma in an attempt to repair the intracranial injury. In this context, one might wish to speculate why healthy newborns who (rather commonly) experience intracranial trauma following passage through the birth canal, resulting in subdural and parenchymal hemorrhage, generally recover asymptomatically (Looney et al., 2007).

### Human Study

Because of the known high concentrations of Compound Ia (7-HOCA) in the chronic subdural hematoma fluid, Applicant analyzed the fluid evacuated from 3 elderly patients with chronic subdurals via LC/MS/MS for the presence of ENT-03.

Patient #1: 66 yo male with 3 weeks of gait instability, headache, difficulty with fine motor movements. CT with 2.2 cm right sided subacute subdural hematoma (SDH) with significant mass effect/midline shift. Indications for drainage were large, symptomatic SDH. Drainage procedure was right sided craniotomy for evacuation of SDH.

Patient #2: 75 yo male who sustained fall 3 weeks prior. Presented with 2-3 days of worsening headache, gait instability, and left lower extremity weakness. CT with bilateral subacute SDH (1.7 cm on right, 1.6 cm on left). Indications for drainage were large, symptomatic SDH. Drainage procedure was bedside twist drill hole craniostomy.

Patient #3: 94 yo male with progressive confusion/altered mental status/gait instability. Work-up demonstrated bilateral subacute on chronic subdural hematomas, both 2.5-3.0 cm in size and with mass effect/brain compression. Indications for drainage were large, symptomatic SDH. Drainage procedure was a bedside twist drill hole craniostomy.

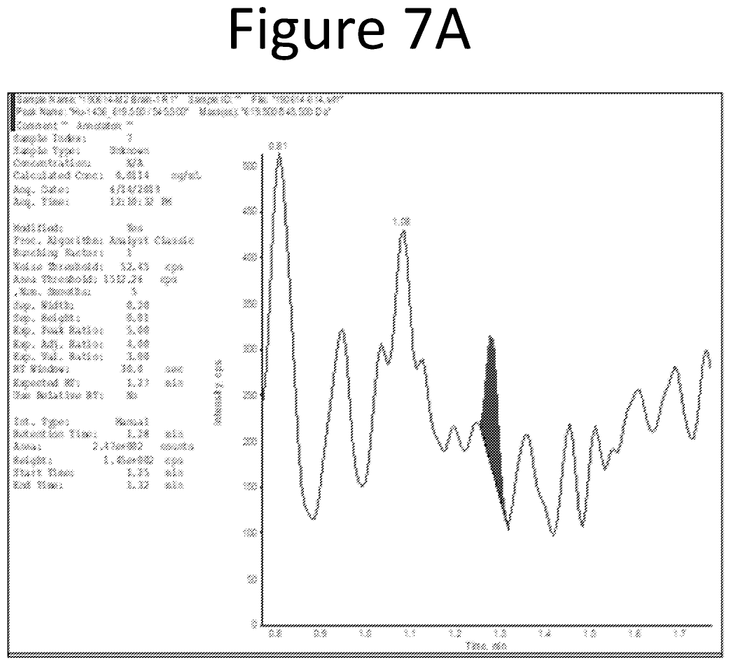

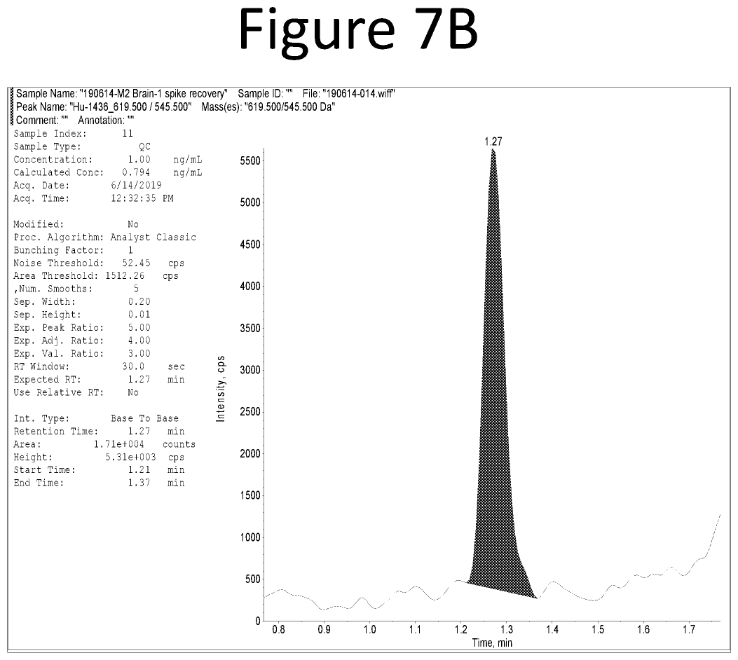

Brain extracts were analyzed via LC/MS/MS for ENT-03. FIG. 7A shows the presence of ENT-03 in brain extract. FIG. 7B shows a reference sample of synthetic ENT-03.

### Mouse Pup Study

Based on these reports Applicant analyzed extracts of brain, liver, and kidneys from newborn mice over the first two postnatal weeks for the presence of ENT-03. 3-oxo-bile acids comprise between 18-40% of the unconjugated bile acids within amniotic fluid during the last trimester of human fetal development (Nakagawa et al. 1990) and are present as a significant percentage of the bile acids in healthy newborn urine, gradually diminishing during the first postnatal month (Wahlen et al., 1989 and Kimura et al., 1999). Since 3-oxo-bile acids are abundant during the newborn period, Applicant focused the search for the putative polyamine-bile acid molecule in neonatal mice. Brain and liver extracts were prepared from mice between day 1 through day 24 of age using a protocol designed to capture ENT-03 based on its physical properties as follows.

Frozen neonatal mice were obtained from Layne Laboratories, and tissues dissected in the frozen state. Tissues were dissected in the frozen state: “pinky” (ld); “large pinky” (2-5d); “small fuzzy” (6-9d); “large fuzzy” (10-14d); “hopper” (15-18d); “small frozen” (18-24d). Tissues (0.5-7 grams) were placed into 4 volumes of methanol containing 0.12N HCl and heated at 80° C. for 5 hours. The tissues were macerated, followed by a brief centrifugation. The supernatant was collected, the volume reduced under a stream of air, and extracted with 1 volume of chloroform/i volume of methanol. The upper phase was reduced in volume and the samples further analyzed by LC/MS/MS.

Synthetic ENT-03 was used as internal standard in LC/MS/MS analysis to permit localization and identification of the corresponding molecule within the chromatographic analysis.

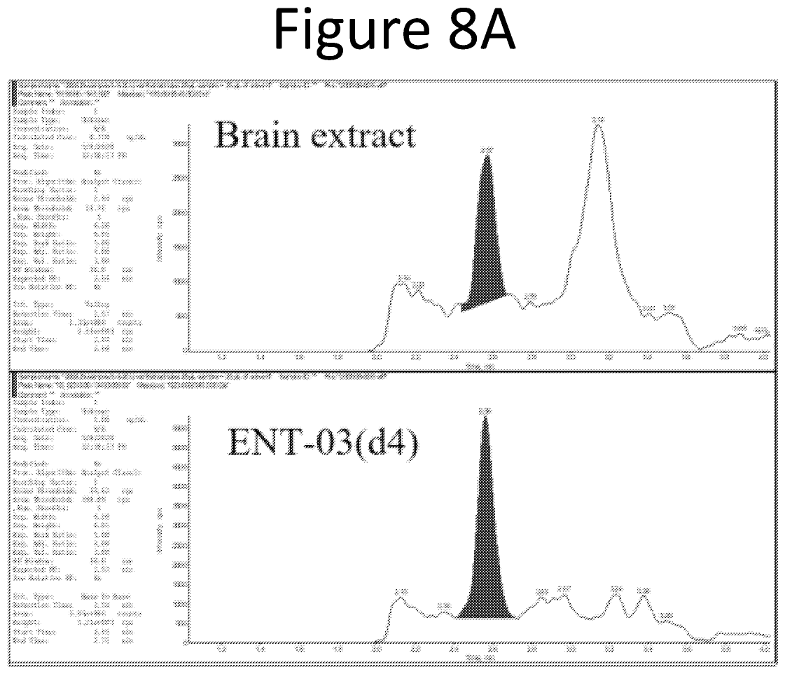

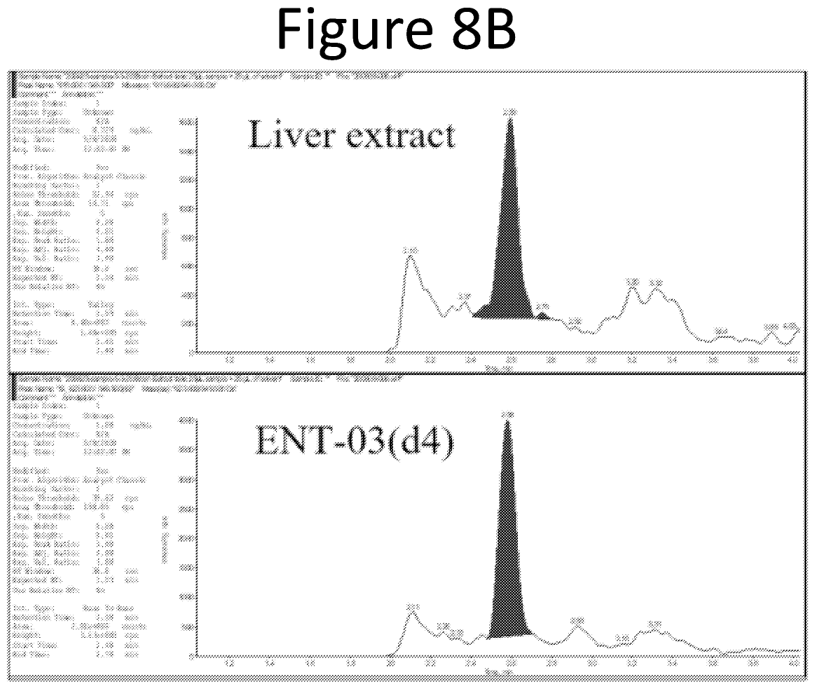

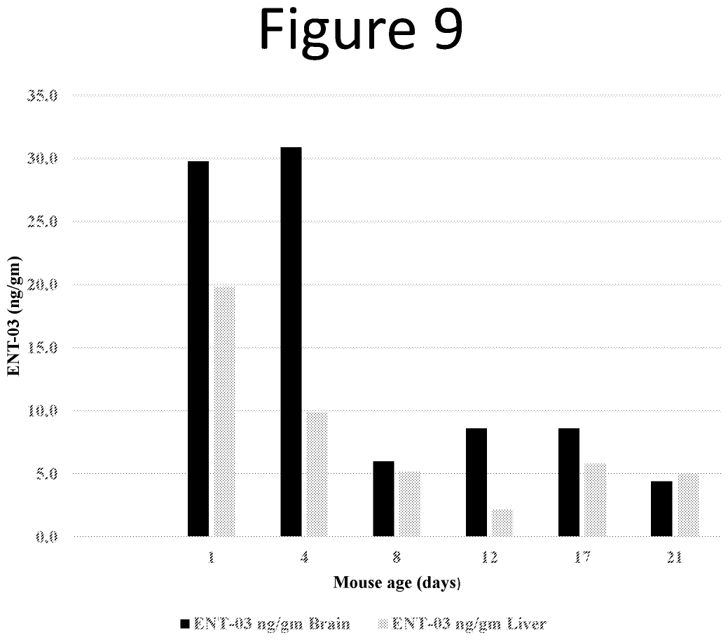

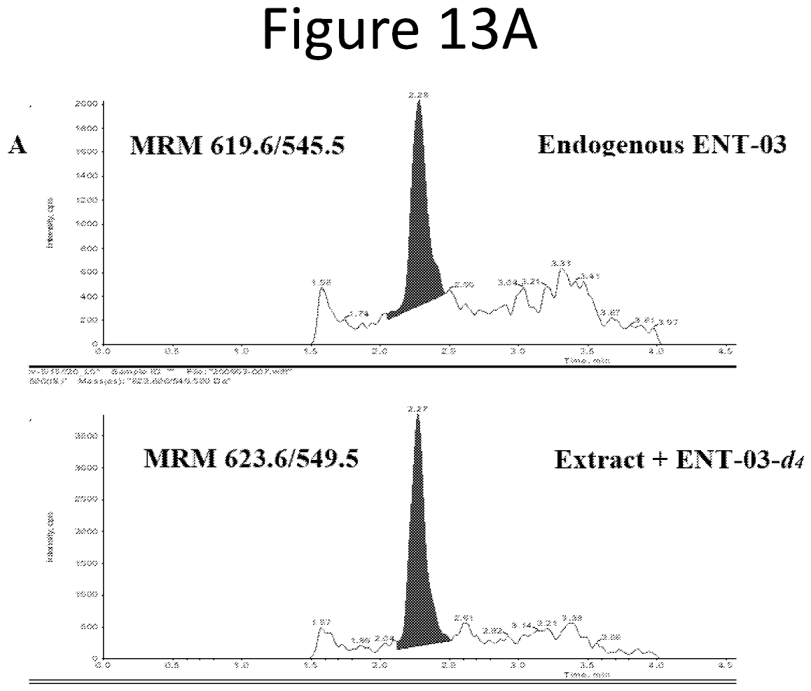

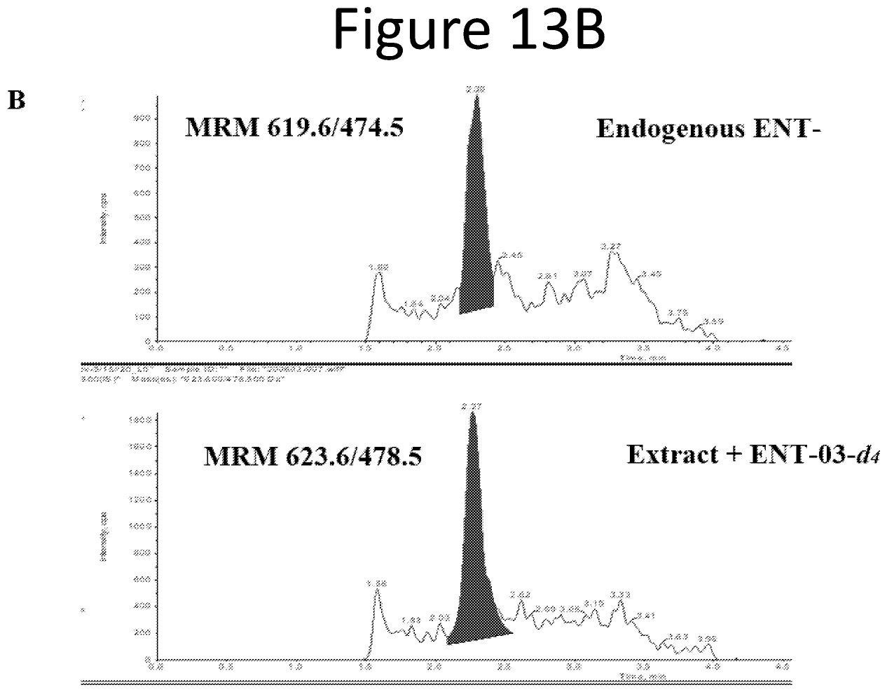

ENT-03 (Compound III) could be detected in brain and liver of neonatal mice (FIGS. 8A and 8B, respectively). The identity of the endogenous ENT-03 was established by its retention time, mass ([M+H⁺]m/z=619.6), and MS/MS MRM of the characteristic fragments of mass 545.57 (FIG. 13A) and 474.39 (FIG. 13B). Approximate concentrations of ENT-03 (Compound III) measured in the brain and liver of neonatal mice over the first 3 weeks of life are presented in FIG. 9. In both brain and liver the highest concentrations appear at birth with a gradual reduction over the following 3 weeks.

Analytical procedures: Tissue extracts were spiked with ENT-03-d₄ and analyzed on a Triple Quad 5500 MS/MS system equipped with a ExionLC™ LC (SCIEX, Redwood City, CA) using Analyst® 1.7.1 software for instrumental control.

Separation of ENT-03 (Compound III) from tissue matrix components was achieved using a Kinetex® 5 μm C18 100 Å 50×2.1 mm chromatographic column (Phenomenex, Torrance, CA, USA). The column was maintained at 25° C., the flow rate was 0.3 mL/min, and the injection volume was 10 μL. The mobile phase (MP) consisted of A: 0.1% formic acid in water and B: 0.1% formic acid in acetonitrile (ACN, v/v). The mobile phase gradient was as follows: after injection, initial conditions with MPA at 80% were held for 0.3 min, decreased to 70% in 1.7 min, to 50% in 0.5 min and held constant for 0.5 min, returning to initial conditions for another 3.5 min of reequilibration time.

Retention time of ENT-03 (Compound III) was approximately 2.3 min and total run time was 7 min. A turbo ion spray interface was used as the ion source, operating in positive ion mode. Acquisition was performed in multiple reaction monitoring (MRM) mode using m/z 619.6 ([M+H]⁺)→545.5 (loss of 74.1 (C₃H₁₀N₂) and m/z 619.6 ([M+H]⁺)→474.4 (loss of 145.2 (C₇H₁₉N₃) at unit resolution. The internal standard was ENT-03-d₄ using MRM of m/z 623.6 ([M+H]⁺)→549.5 or m/z 623.6 ([M+H]⁺)→478.5. Ion spray voltage was 4500 V and collision energy was 55 (619.6/474.5) or 43 (619.5/545.5) V. Declustering potential was 70V. The collision gas was nitrogen and the ion spray temperature was 650° C. The MRM transitions were confirmed with a mass error of 10 ppm or less using high-resolution MRM on a X500 QTOF System (SCIEX).

For ENT-03 (Compound III) tissue level estimation, calibration curves were prepared from ENT-03 chromatographic peak areas ratios to the internal standard and using linear regression with a (1/x²) weighting factor that was chosen based on goodness-of-fit criteria, including coefficient of determination (r²), the back-calculated concentration of individual calibrators, and minimization of the intercept value.

### Example 8: ENT-05 and Regulatory Phosphatase Inhibition

The purpose of this example was to evaluate the inhibition of regulatory phosphatases by ENT-05.

ENT-05 was synthesized and assayed against the bank of regulatory phosphatases.

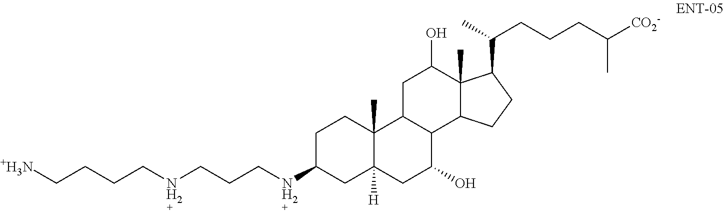

ENT-05, which differs from ENT-03 (Compound III) with respect to the polyamine, and the presence of a hydroxyl group on C12, exhibits inhibitory activity with great specificity against the proto-oncogene PTPN11 (E76K), as shown in Tables 18 Å and 18B below.

TABLE 18A

| | | | PP2A | | | | |
|---|---|---|---|---|---|---|---|
| | | | Alpha/ | | | | |
| | | | PP2R1A | PTPN6/ | PTPN11/ | PTPRC/ | DUSP22/ |
| Compound ID | PP1B | PP1A | Complex | SHP1 | SHP | CD45 | MKPX |
| ENT-05 | >1.00E−04 | >1.00E−04 | 3.80E−06 | >1.00E−04 | 1.84E−05 | >1.00E−04 | **>1.00E−04 |
| PTP1BInhibitor | 5.99E−06 | 6.91E−06 | ND | 3.47E−06 | 2.31E−06 | 2.80E−05 | 9.63E−06 |
| Canthardic Acid | ND | ND | 2.785E−08 | ND | ND | ND | ND |
| SHP099 | ND | ND | ND | ND | ND | ND | ND |

TABLE 18B

| | PTPN2/TC- | PTPN7/LC- | PTPN12/PTP- | PTPN1/PTP1B- | PN11/ | PTPN11/SHP2- |
|---|---|---|---|---|---|---|
| Compound ID | PTP | PTP | PEST | CD | SHP2 | FL(E76K) |
| ENT-05 | >1.00E−04 | 3.97E−06 | **>1.00E−04 | 2.58E−06 | 9.76E−07 | 4.30E−08 |
| PTP1BInhibitor | 7.53E−06 | 8.65E−06 | 2.16E−06 | 6.23E−06 | ND | ND |
| Canthardic Acid | ND | ND | ND | ND | ND | ND |
| SHP099 | ND | ND | ND | ND | 1.75E−08 | 1.66E−06 |

As another example, ENT-06 was synthesized.

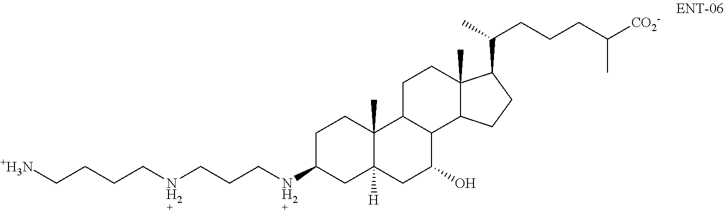

ENT-06 differs from ENT-03 in the substitution of a spermidine for a spermine. ENT-06 exhibits the following inhibitory activity against the human regulatory phosphatases (Tables 19A and 19B).

TABLE 19A

| | | | PP2A | | | | |
|---|---|---|---|---|---|---|---|
| | | | Alpha/ | | | | |
| | | | PP2R1A | PTPN6/ | PTPN11/ | PTPRC/ | DUSP22/ |
| Compound ID | PP1B | PP1A | Complex | SHP1 | SHP | CD45 | MKPXPT |
| ENT-03 | >1.00E−04 | >1.00E−04 | >1.00E−04 | >1.00E−04 | 5.00E−06 | 5.17E−07 | 1.38E−06 |
| ENT-06 | >1.00E−04 | >1.00E−04 | >1.00E−04 | >1.00E−04 | 3.45E−06 | 1.22E−06 | 2.09E−06 |
| PTP1BInhibitor | 4.97E−06 | 6.45E−06 | ND | 3.67E−06 | 2.12E−06 | 2.66E−05 | 7.23E−06 |
| Canthardic Acid | ND | ND | 3.01E−08 | ND | ND | ND | ND |
| SHP099 | ND | ND | ND | ND | ND | ND | ND |

TABLE 19B

| | PTPN2/TC- | PTPN7/LC- | PTPN12/PTP- | PTPN1/PTP1B- | PN11/ | PTPN11/SHP2- |
|---|---|---|---|---|---|---|
| Compound ID | PTP | PTP | PEST | CD | SHP2 | FL(E76K) |
| ENT-03 | >1.00E−04 | 6.34E−07 | 8.61E−07 | 1.17E−07 | 1.42E−06 | 2.94E−07 |
| ENT-06 | >1.00E−04 | 1.05E−06 | 8.62E−07 | 1.47E−07 | 3.17E−06 | 3.45E−07 |
| PTP1BInhibitor | 5.86E−06 | 6.89E−06 | 1.21E−06 | 5.10E−06 | ND | ND |
| Canthardic Acid | ND | ND | ND | ND | ND | ND |
| SHP099 | ND | ND | ND | ND | 2.53E−08 | 1.58E−06 |

As can be seen in this example, the substitution of a spermidine for a spermine does not change the inhibitory profile of ENT-03. This example also teaches that the C-12 moiety on ENT-05 (above) drastically alters the specificity of the molecule towards the human regulatory phosphatases.

Phosphatase assays used: The assays used were from Recation Biology Inc. PTPN11/SHP2-FL: Recombinant human PTPN11 full length (Genbank accession #NM_00133043.1; aa 2-597, isoform 1 (canonical)) was expressed in E. Coli with N-terminal StrepII-TEV, C-terminal His-tag. Mw=71.93 kDa.

PTPN11/SHP2 (E76K)-FL: Recombinant human PTPN11 full length (Genbank accession #NM_00133043.1; aa 2-597, isoform 1 (canonical)) with E76K mutation was expressed in E. Coli with N-terminal StrepII-TEV, C-terminal His-tag. Mw=71.93 kDa.

Activation peptide: H2N-LN(pY)IDLDLV(dPEG8)LST(pY)ASINFQK-amide (Fortanet et al., 2016) Substrate: DiFMUP [6,8-difluoro-7-hydroxy-4-methylcoumarin]Final concentration in the assay: For wild type: 0.35 M Activating peptide; 100 M DiFMUP; for mutant: No Activating peptide; 100 μM DiFMUP Assay buffer: 60 mM HEPES (pH 7.4), 1 mM EDTA, 75 mM KCl, 75 mM NaCl, 0.01% Brij-35, 5 mM DTT, and 10% DMSO (final).

Other phosphatases assayed: Assay buffer: 25 mM HEPES (pH 7.5), 5 mM MgCl₂, 0.01% Brij-35, 1 mM DTT, and 1% DMSO. For PP2A Alpha/PPP2R1A Complex, PP1A, and PP1B 1 mM MnCl₂ was added to the assay buffer. The concentration of DiFMUP varied with the phosphatase chosen: 2 M for PTPN1/PTP1B-CD; 30 M for PP1B; 10 μM for all other phosphatases. The phosphatase inhibitors were PTP1B CAS 765317-72-4 (Sigma Aldrich cat #539741); Cantharidic acid (Santa Cruz Biotech, cat #sc-201323); SHP009 (ChemieTek, cat #CT-SHP099).

General procedure: Enzyme and substrate are freshly prepared in assay buffer; the enzyme solution is introduced into the reaction well, followed by delivery of the inhibitors in 100% DMSO via acoustic technology (Echo550; nanoliter range); the reaction is incubated for 20 min at room temperature; substrate is then delivered into the reaction well to initiate the reaction; the enzyme activities are monitored (Ex/Em 355/460) by an increase in fluorescence for 120 min at room temperature. The slope (signal/min) of the linear portion of time course is determined and the rates calculated in the presence of inhibitor relative to the DMSO control.

### Example 9: ENT-03 Stimulates the Transcription of Genes Involved in Red Blood Cell Production and Immune Cell Function in the Spleen of Aged Mice

As in humans, the spleen of the mouse plays a primary immune function (Smith et al., 2019). In the adult mouse, however, the spleen can become a red blood cell forming organ in a variety of experimental settings (Morita et al., 2011), although it normally cedes this function to the bone marrow during the first few weeks of postnatal life (Wolber et al., 2002).

To explore the effects of ENT-03 (Compound III) on the hematopoietic and immune systems of the spleens of young and aged mice, the transcriptomes of the spleens of the animals described in the previous rejuvenation of gut transcriptome Example were studied.

The transcriptional response to ENT-03 (Compound III) in the aged spleen (Table 20, below) was more intense than in the spleen of the younger animals (Table 21, below). 109 genes in the aged spleen were significantly upregulated by >1.5 fold, compared with 4 genes in the young. The genes most robustly induced in the old spleen are responsible for erythropoiesis (italicized rows in Table 20) and immune functions (bold rows in Table 20). The genes involved in hemoglobin synthesis are at the top of the list (hemoglobin α and β proteins, aminolevulinic acid synthase) along with abundant proteins involved in the structure/function of the red blood cell (the chloride-bicarbonate exchanger, band 4.2 protein, and 2,3 biphosphoglycerate mutase). These data are compatible with the hypothesis that ENT-03 is stimulating a program in the aged mouse normally operational during the first few weeks of postnatal life.

Generally speaking, the immune genes induced by ENT-03 correspond to those of the innate arm. These include IL-21, secreted by T cells and NKT cells, which has broad stimulatory effects across the breadth of the innate immune system (Spolski et al., 2014); and the IL22 binding protein (IL22rα2), secreted by numerous innate immune cells, that helps curb the action of the proinflammatory cytokine IL22 following resolution of an infection (Huber et al., 2012) reducing the probability of post-inflammatory tumorigenesis (Huber et al., 2012). Other highly induced genes encode for receptors that are expressed by dendritic cells and macrophages, such as DC-SIGN (CD209) which plays a key role in the recognition by dendritic cells of viruses and other pathogens (Svajger et al., 2010); the macrophage chemokine receptor CCR2, which plays a critical role in the normal reparative response to tissue injury (Boniakowski et al., 2018); and the Fc alpha/mu receptor (Fcamr) that mediates endocytosis of IgM coated pathogens by the macrophage (Shibuya et al., 2000).

None of the splenic genes highly induced by ENT-03 in the aged spleen appear in the set of responsive genes in the spleen of young animals.

TABLE 20
Transcriptional response in ENT-03 treated vs. untreated aged spleen

| | | log2FoldΔ | | |
|---|---|---|---|---|
| | | (treated vs | | |
| Gene | Chrom | untreated) | padj | gene description |
| Hbb-bt | chr7 | 3.7347 | 1.0409E−18 | hemoglobin_beta_adult_t_chain |
| Hbb-bs | chr7 | 3.7096 | 3.6524E−17 | hemoglobin_beta_adult_s_chain |
| Hba-a1 | chr11 | 3.3063 | 2.5751E−12 | hemoglobin_alpha_adult_chain_1 |
| Alas2 | chrX | 3.1518 | 5.5197E−10 | aminolevulinic_acid_synthase_2_erythroid |
| Apol11b | chr15 | 2.866 | 1.2774E−06 | apolipoprotein_L_11b |
| Snca | chr6 | 2.7302 | 6.7702E−08 | synuclein_alpha |
| Il21 | chr3 | 2.6995 | 1.4958E−07 | interleukin_21 |
| Trim10 | chr17 | 2.6019 | 1.4584E−06 | tripartite_motif-containing_10 |
| Slc4a1 | chr11 | 2.4837 | 0.000018554 | solute_carrier_family_4_(anion_exchanger)_member_1 |
| Crisp3 | chr17 | 2.4801 | 0.00001087 | cysteine-rich_secretory_protein_3 |
| BC094916 | chr1 | 2.4206 | 1.6453E−12 | cDNA_sequence_BC094916 |
| Klra13-ps | chr6 | 2.2739 | 0.000055312 | killer_cell_lectin- |
| | | | | like_receptor_subfamily_A_member_13 |
| Ms4a4c | chr19 | 2.2629 | 1.4648E−06 | membrane-spanning_4- |
| | | | | domains_subfamily_A_member_4C |
| Gm15674 | chr1 | 2.2616 | 0.00025227 | predicted_gene_15674 |
| Fcamr | chr1 | 2.26 | 0.00023611 | Fc_receptor_IgA_IgM_high_affinity |
| Cd209a | chr8 | 2.2387 | 9.2759E−08 | CD209a_antigen |
| Il22ra2 | chr10 | 2.1757 | 0.000018994 | interleukin_22_receptor_alpha_2 |
| Klri1 | chr6 | 2.1282 | 0.00021207 | killer_cell_lectin-like_receptor_family_I_member_1 |
| Apo17c | chr15 | 2.1187 | 1.9578E−07 | apolipoprotein_L_7c |
| S100a4 | chr3 | 2.0775 | 1.1352E−06 | S100_calcium_binding_protein_A4 |
| Dscaml1 | chr9 | 2.0299 | 0.0013572 | Down_syndrome_cell_adhesion_molecule_like_1 |
| Angptl1 | chr1 | 2.0064 | 0.0017673 | angiopoietin-like_1 |
| Gm4841 | chr18 | 1.993 | 0.0016464 | predicted_gene_4841 |
| Bex6 | chr16 | 1.9805 | 0.001294 | brain_expressed_gene_6 |
| Epb4.2 | chr2 | 1.9538 | 0.0024314 | erythrocyte_protein_band_4.2 |
| Gm15056 | chr8 | 1.9402 | 0.00074293 | predicted_gene_15056 |
| Gm15848 | chr1 | 1.9395 | 0.0014089 | predicted_gene_15848 |
| RP23- | chr12 | 1.9042 | 0.0034892 | predicted_gene_29361 |
| 291K4.3 | | | | |
| Slfn4 | chr11 | 1.9041 | 0.0033094 | schlafen_4 |
| Erv3 | chr2 | 1.9021 | 0.00041989 | endogenous_retroviral_sequence_3 |
| Adipoq | chr16 | 1.8937 | 0.0037472 | adiponectin_C1Q_and_collagen_domain_containing |
| Ccr2 | chr9 | 1.8781 | 0.00066331 | chemokine_(C—C_motif)_receptor_2 |
| Gm16170 | chr11 | 1.8443 | 0.0018004 | predicted_gene_16170 |
| Hba-a2 | chr11 | 1.8432 | 0.0050296 | hemoglobin_alpha_adult_chain_2 |
| Chordc1 | chr9 | 1.8282 | 0.000022877 | cysteine_and_histidine-rich_domain_(CHORD)- |
| | | | | containing_zinc-binding_protein_1 |
| Ms4a4b | chr19 | 1.8267 | 0.00074293 | membrane-spanning_4− |
| | | | | domains_subfamily_A_member_4B |
| Ceacam2 | chr7 | 1.8202 | 0.0019663 | carcinoembryonic_antigen- |
| | | | | related_cell_adhesion_molecule_2 |
| Trim59 | chr3 | 1.8191 | 0.00051144 | tripartite_motif-containing_59 |
| A430104N | chr11 | 1.7777 | 0.0012186 | Mir142_host_gene_(non-protein_coding) |
| 18Rik | | | | |
| H3f3c | chr2 | 1.7761 | 0.006137 | H3_histone_family_3C |
| Gpr174 | chrX | 1.7489 | 0.0013772 | G_protein-coupled_receptor_174 |
| Ccl22 | chr8 | 1.7457 | 0.0017096 | chemokine_(C—C_motif)_ligand_22 |
| Bpgm | chr6 | 1.7452 | 0.0012861 | 23-bisphosphoglycerate_mutase |
| 9130020K | chr4 | 1.744 | 0.0086611 | RIKEN_cDNA_9130020K20_gene |
| 20Rik | | | | |
| 4632427E1 | chr7 | 1.736 | 0.0051337 | RIKEN_cDNA_4632427E13_gene |
| 3Rik | | | | |
| Mndal | chr1 | 1.7343 | 0.00029797 | myeloid_nuclear_differentiation_antigen_like |
| Klra10 | chr6 | 1.7308 | 0.0092544 | killer_cell_lectin- |
| | | | | like_receptor_subfamily_A_member_10 |
| Cr2 | chr1 | 1.7172 | 0.000057646 | complement_receptor_2 |
| Chil5 | chr3 | 1.715 | 0.00659 | chitinase-like_5 |
| Art2a-ps | chr7 | 1.714 | 0.000026233 | ADP-ribosyltransferase_2a_pseudogene |
| Gm18853 | chr7 | 1.7076 | 0.010528 | predicted_gene_18853 |
| Samd91 | chr6 | 1.6916 | 0.0025867 | sterile_alpha_motif_domain_containing_9-like |
| Kmo | chr1 | 1.6871 | 3.5618E−07 | kynurenine_3-monooxygenase_(kynurenine_3- |
| | | | | hydroxylase) |
| Nudt17 | chr3 | 1.6761 | 0.0062584 | nudix_(nucleoside_diphosphate_linked_moiety_X)- |
| | | | | type_motif_17 |
| Pyhin1 | chr1 | 1.6712 | 0.0016474 | pyrin_and_HIN_domain_family_member_1 |
| Gm14446 | chr19 | 1.6636 | 7.5769E−09 | predicted_gene_14446 |
| Gm16754 | chr9 | 1.663 | 0.013156 | predicted_gene_16754 |
| Skint3 | chr4 | 1.6602 | 0.0066421 | selection_and_upkeep_of_intraepithelial_T_cells_3 |
| Gm15382 | chr15 | 1.6598 | 0.013129 | predicted_gene_15382 |
| Gm14539 | chrX | 1.6584 | 0.011189 | predicted_gene_14539 |
| B3galt2 | chr1 | 1.6529 | 0.012914 | UDP-Gal:betaGlcNAc_beta_13- |
| | | | | galactosyltransferase_polypeptide_2 |
| Gm20506 | chr17 | 1.6511 | 0.0055872 | predicted_gene_20506 |
| Trbv3 | chr6 | 1.6449 | 0.00075871 | T_cell_receptor_beta_variable_3 |
| Gvin1 | chr7 | 1.6426 | 0.0030561 | GTPase_very_large_interferon_inducible_1 |
| Gapdhs | chr7 | 1.6401 | 0.012163 | glyceraldehyde-3-phosphate_dehydrogenase_spermatogenic |
| Igkv10-95 | chr6 | 1.6339 | 0.004547 | immunoglobulin_kappa_variable_10-95 |
| Tgtp1 | chr11 | 1.6241 | 8.2575E−07 | T_cell_specific_GTPase_1 |
| Tnfrsf4 | chr4 | 1.6208 | 6.0546E−08 | tumor_necrosis_factor_receptor_superfamily_member_4 |
| Gpr171 | chr3 | 1.6192 | 2.4193E−06 | G_protein-coupled_receptor_171 |
| Gm12359 | chr11 | 1.612 | 0.017289 | predicted_gene_12359 |
| Tespa1 | chr10 | 1.6091 | 2.5415E−07 | thymocyte_expressed_positive_selection_associated_1 |
| Cxcr6 | chr9 | 1.6084 | 0.00074332 | chemokine_(C—X—C_motif)_receptor_6 |
| Gm9733 | chr3 | 1.6052 | 0.0033127 | predicted_gene_9733 |
| Phf11a | chr14 | 1.5879 | 0.000072239 | PHD_finger_protein_11A |
| Bcl2l14 | chr6 | 1.5821 | 0.0054825 | BCL2-like_14_(apoptosis_facilitator) |
| Wfikkn1 | chr17 | 1.5812 | 0.019127 | WAP_FS_Ig_KU_and_NTR-containing_protein_1 |
| Gm12064 | chr11 | 1.5812 | 0.017839 | predicted_gene_12064 |
| Stat4 | chr1 | 1.5777 | 0.00028359 | signal_transducer_and_activator_of_transcription_4 |
| Gimaplos | chr6 | 1.5766 | 0.000022374 | GTPase_IMAP_family_member_1_opposite_strand |
| AI504432 | chr3 | 1.5764 | 0.00043427 | expressed_sequence_AI504432 |
| Ddx43 | chr9 | 1.5727 | 0.014119 | DEAD_(Asp-Glu-Ala-Asp)_box_polypeptide_43 |
| Cyp4b1- | chr4 | 1.5655 | 0.02129 | cytochrome_P450_family_4_subfamily_b_polypeptide_1_ |
| ps2 | | | | pseudogene_2 |
| Hsph1 | chr5 | 1.5639 | 5.0637E−09 | heat_shock 105 kDa/110 kDa_protein_1 |
| Gm26613 | chr11 | 1.5638 | 0.017312 | predicted_gene_26613 |
| Phf11c | chr14 | 1.562 | 0.00010438 | PHD_finger_protein_11C |
| Mterf1a | chr5 | 1.5619 | 0.0012861 | mitochondrial_transcription_termination_factor_1a |
| Igkv4-74 | chr6 | 1.5563 | 0.022022 | immunoglobulin_kappa_variable_4-74 |
| Ifi203 | chr1 | 1.5516 | 0.00025302 | interferon_activated_gene_203 |
| Hbq1b | chr11 | 1.5454 | 0.020239 | hemoglobin_theta_1B |
| Insm1 | chr2 | 1.5427 | 0.0010054 | insulinoma-associated_1 |
| Plac8 | chr5 | 1.5426 | 6.9197E−14 | placenta-specific_8 |
| Gad1-ps | chr10 | 1.5374 | 0.019744 | glutamate_decarboxylase_1_pseudogene |
| Cdcp1 | chr9 | 1.5358 | 0.00036257 | CUB_domain_containing_protein_1 |
| Ly6d | chr15 | 1.5352 | 8.0833E−10 | lymphocyte_antigen_6_complex_locus_D |
| Serpinale | chr12 | 1.5291 | 0.015891 | serine_(or_cysteine)_peptidase_inhibitor_clade_A_member_1E |
| Gpr82 | chrX | 1.5279 | 0.024478 | G_protein-coupled_receptor_82 |
| Gm10521 | chr1 | 1.5248 | 0.0029372 | predicted_gene_10521 |
| Stap1 | chr5 | 1.5241 | 0.00044727 | signal_transducing_adaptor_family_member_1 |
| Ms4a1 | chr19 | 1.5233 | 0.0019425 | membrane-spanning_4-domains_subfamily_A_member_1 |
| Bzrap1 | chr11 | 1.5216 | 0.020864 | benzodiazepine_receptor_associated_protein_1 |
| Il4i1 | chr7 | 1.521 | 0.000013199 | interleukin_4_induced_1 |
| Tgtp2 | chr11 | 1.5203 | 2.163E−08 | T_cell_specific_GTPase_2 |
| Capsl | chr15 | 1.5194 | 0.02621 | calcyphosine-like |
| 4631405J1 | chr2 | 1.5184 | 0.02629 | RIKEN_cDNA_4631405J19_gene |
| 9Rik | | | | |
| A430110L | chr1 | 1.5155 | 0.025229 | RIKEN_cDNA_A430110L20_gene |
| 20Rik | | | | |
| Malat1 | chr19 | 1.51 | 0.0013738 | metastasis_associated_lung_adenocarcinoma_transcript_1_ |
| | | | | (non-coding_RNA) |
| Asprv1 | chr6 | 1.508 | 0.0062434 | aspartic_peptidase_retroviral-like_1 |
| Ighj4 | chr12 | 1.507 | 0.000033775 | immunoglobulin_heavy_joining_4 |
| Spo11 | chr2 | 1.5059 | 0.016958 | SPO11_meiotic_protein_covalently_bound_to_DSB homolog_ |
| | | | | (S._cerevisiae) |

TABLE 21
Transcriptional response in ENT-03 treated vs. untreated young spleen

| GeneName | Chrom | log2FoldA | padj | Gene description |
|---|---|---|---|---|
| BC147527 | chr13 | 1.5492 | 5.13E−09 | cDNA_sequence_BC147527 |
| Actr6 | chr10 | 1.5441 | 5.13E−09 | ARP6_actin-related_protein_6 |
| Gt(ROSA)26S | chr6 | 1.5188 | 0.0043575 | gene_trap_ROSA_26_Philippe_Soriano |
| or | | | | |
| Lsm7 | chr10 | 1.5096 | 1.164E−06 | LSM7_homolog_U6_small_nuclear_RNA_ |
| | | | | associated_(S._cerevisiae) |

### Example 10: Treatment Induced Gene Expression in Mouse Stomach Tissues, Comparison of ENT-03 and ENT-02 (MSI-1436)

The purpose of this example is to identify transcriptional changes between young and old mice and compare the effects of ENT-02 (MSI-1436) treatments on gene expression with those of ENT-03. Mice were treated with ENT-03, ENT-02 (MSI-1436) or a vehicle control. Samples of stomach tissues from the mice were analysed by RNA-sequencing on an Illumina platform.

The aims of this analysis were to: identify transcriptional changes between young and old mice; determine if ENT-01 or ENT-02 (MSI-1436) treatments could reverse those age-related changes; and compare the effects of ENT-02 (MSI-1436) treatments on gene expression with those of ENT-03.

To achieve these aims, data were subject to a quality control evaluation, differential expression and functional enrichment analyses, as well as a congruence analysis. Data for ENT-ENT-03 treatments were compared to the results generated for ENT-02 (MSI-1436).

To identify those genes that were significantly differentially expressed between groups, an arbitrary threshold was applied based on fold changes in expression. A four-fold change in expression between groups was used as a measure of pseudo-significance in the absence of replicate samples. At this threshold, differentially expressed genes were identified in all contrasts. For gene expression changes associated with ageing, or ENT-02 (MSI-1436) treatments in young mice, the proportions of down-regulated genes (72-80%) were higher than the proportions of upregulated genes (20-28%). The trend was opposite for both treatments in old mice, where 82-89% of gene expression changes were up-regulation and 12-18% were down-regulation.

When comparing differentially expressed genes across contrasts, it was apparent that genes downregulated in aged mice overlapped significantly with genes up-regulated in response to ENT-02 treatment in old mice (hypergeometric P<0.0001). To a lesser extent, there was also significant overlap in genes down-regulated in aged mice and up-regulated in response to treatment in old mice. In young mice, genes that were down-regulated upon ENT-02 treatment significantly overlapped with the down-regulated genes associated with ageing. This result was also true for genes up-regulated in both contrasts.

For each comparison, the number of sample genes significant at various statistical thresholds and fold change 4 were tallied. As mentioned previously, for statistical robustness, only those with an adjusted p-value <0.05 should be considered.

The following statistical significance threshold was chosen to define differentially expressed genes: • fold change≥4

TABLE 22
Summary of effect size evaluations: The number of significant features in each
comparison at a range of effect size thresholds. Each row relates to a single comparison,
while each column relates to a fold change threshold

| Contrast | Direction | Any | 1.3x | 2x | 4x | 8x | 16x |
|---|---|---|---|---|---|---|---|
| ENT-01 vs control (young) | Up-regulated | 7460 | 1430 | 232 | 58 | 38 | 28 |
| ENT-01 vs control (young) | Down-regulated | 7553 | 2367 | 642 | 144 | 47 | 12 |
| ENT-02 (MSI-1436) vs control | Up-regulated | 7346 | 1860 | 232 | 30 | 7 | 1 |
| (young) | | | | | | | |
| ENT-02 (MSI-1436) vs control | Down-regulated | 7667 | 2429 | 531 | 120 | 43 | 16 |
| (young) | | | | | | | |
| ENT-01 vs control (old) | Up-regulated | 7553 | 2430 | 766 | 317 | 168 | 116 |
| ENT-01 vs control (old) | Down-regulated | 7460 | 2235 | 431 | 66 | 25 | 11 |
| ENT-02 (MSI-1436) vs control | Up-regulated | 7416 | 2109 | 657 | 298 | 139 | 42 |
| (old) | | | | | | | |
| ENT-02 (MSI-1436) vs control | Down-regulated | 7597 | 1395 | 203 | 38 | 7 | 1 |
| (old) | | | | | | | |
| Old vs young (control) | Up-regulated | 7386 | 1715 | 312 | 76 | 28 | 11 |
| Old vs young (control) | Down-regulated | 7627 | 2486 | 734 | 218 | 85 | 39 |
| Old vs young (ENT-01) | Up-regulated | 7479 | 1647 | 559 | 263 | 178 | 135 |
| Old vs young (ENT-01) | Down-regulated | 7534 | 1606 | 267 | 69 | 36 | 27 |
| Old vs young (ENT-02 | Up-regulated | 7669 | 2644 | 676 | 190 | 78 | 39 |
| (MSI-1436)) | | | | | | | |
| Old vs young (ENT-02 | Down-regulated | 7344 | 1986 | 314 | 63 | 8 | 4 |
| (MSI-1436)) | | | | | | | |

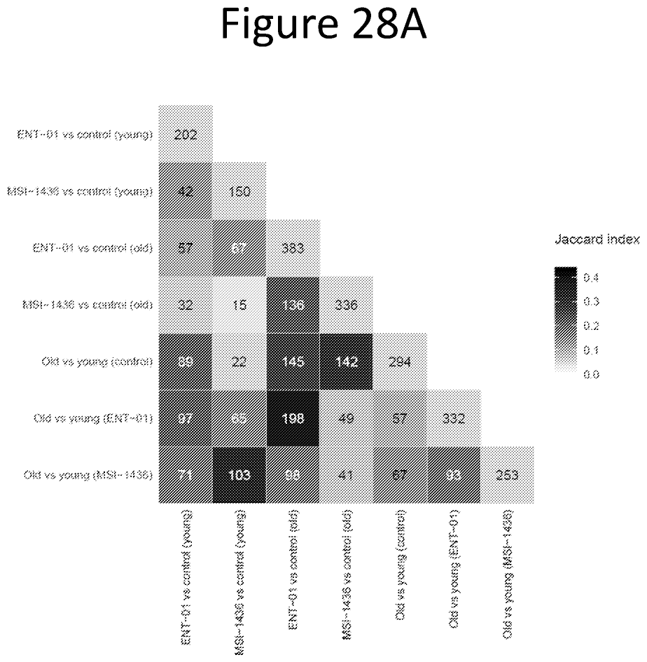

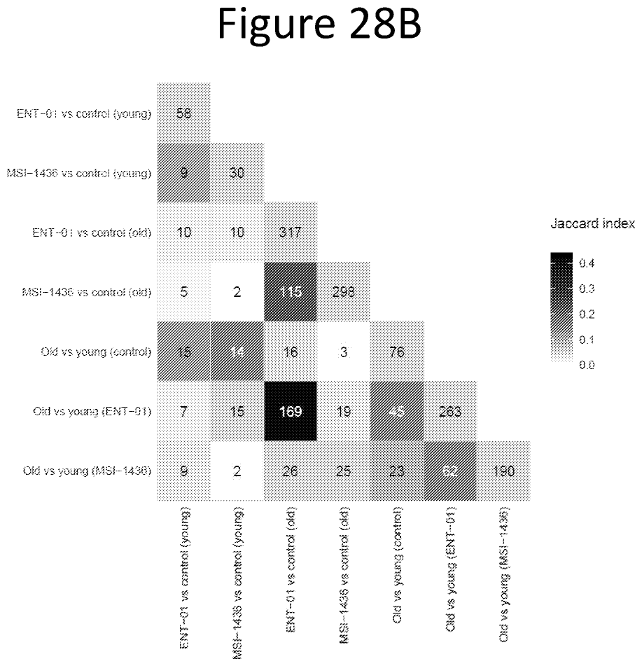

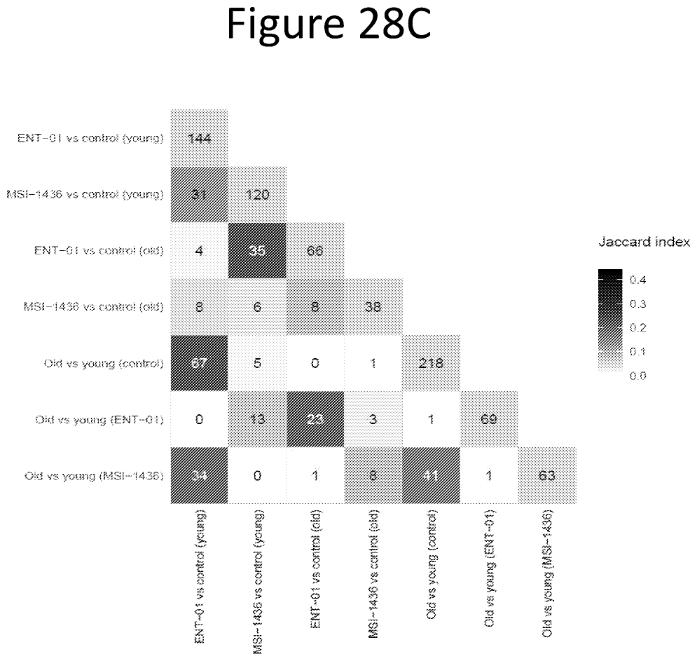

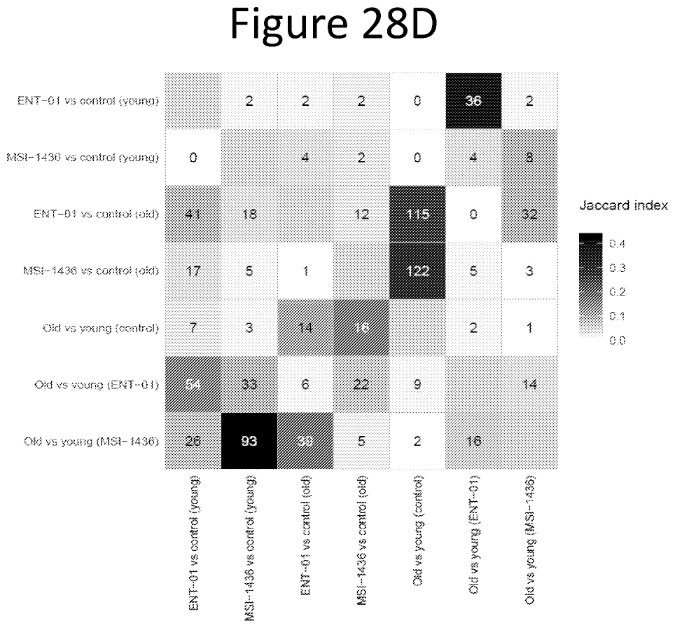

To investigate the overlap between selected genes from the multiple contrasts performed, the number of overlapping differentially expressed genes were counted (defined using fold change 4) between all pairwise combinations of the comparisons performed. The amount of overlap is represented in FIGS. 28A-28D. For each comparison, the value in the plot represents the number of intersecting selected genes and the color represents the Jaccard index (the intersection over the union) for the two contrasts under consideration.

To investigate the overlap between significant genes from the contrasts performed, a set of scatter plots was generated comparing the fold change between pairs of contrasts. Functional enrichment analysis was performed upon those genes where expression differences were greater than four-fold in individual contrasts. Reactome and GO term databases were interrogated to identify relevant terms that were significantly enriched in differentially expressed genes (enrichment P<0.05). Pathways related to the immune system such as platelet degranulation, antimicrobial peptides and complement cascade were generally amongst the most enriched pathways across the contrasts. Pathways such as keratinisation and keratinocyte differentiation were enriched in genes changed upon ageing or ENT-02 (MSI-1436) treatments in old mice. Pathways such as muscle contractions and sarcomere organisation were enriched in genes changed upon ageing, but were also enriched in ENT-02 treatment-affected genes in young mice.

Significant genes (at fold change 4) from each contrast were analysed for enrichment of Reactome pathway membership using a hypergeometric test by mapping genes to genes (if appropriate). Enrichment (p-value <0.05) was assessed for the union of selected genes.

Genes that were differentially expressed in response to ENT-02 treatment, as identified in this study, were compared to those that were associated with ENT-03 treatments, respectively. ENT-03 treatment-associated gene expression changes were identified previously.

Across all contrasts at the relevant statistical thresholds used to identify significant features, there were ten or less genes that changed in expression upon both treatments (ENT-02 and ENT-03). Note that this included the gene expression changes associated with ageing. Specifically, the expression of only one gene, Sypl2, was changed significantly between the old and young mice of both studies.

Comparisons of the genes which were down-regulated in ageing and up-regulated in treatment revealed that genes affected by ENT-02-treatment did not overlap with those affected by ENT-03.

Congruence analysis was performed. Specifically, the analysis compared the effects of human- or shark-origin compound analogues on murine stomach tissue gene expression profile as follows: ENT-02 (MSI-1436) was compared to ENT-03.

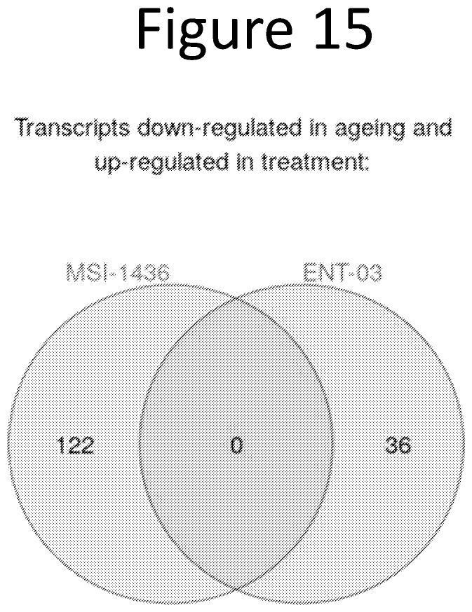

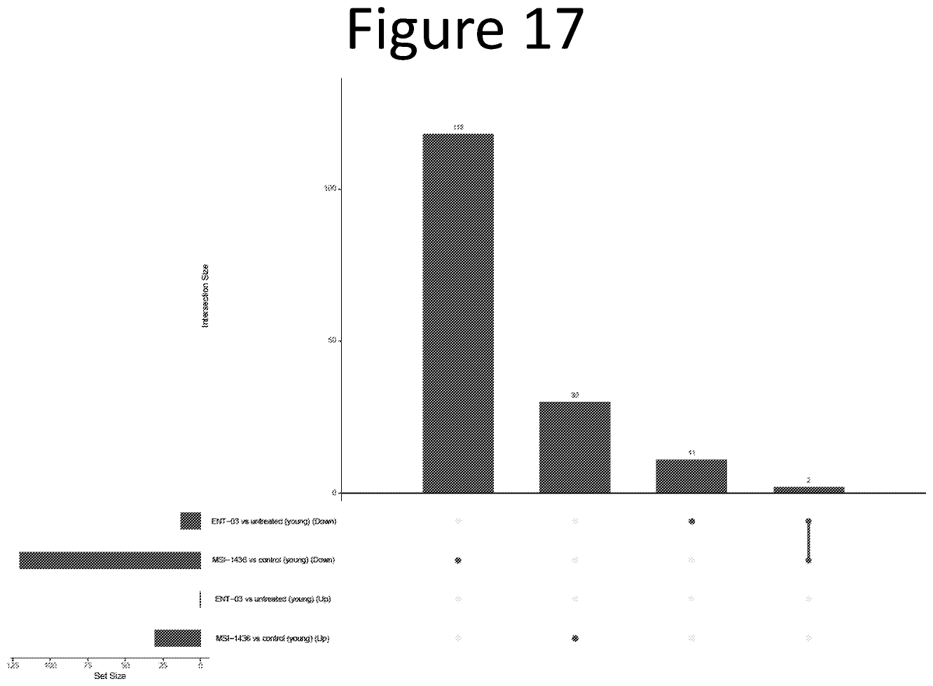

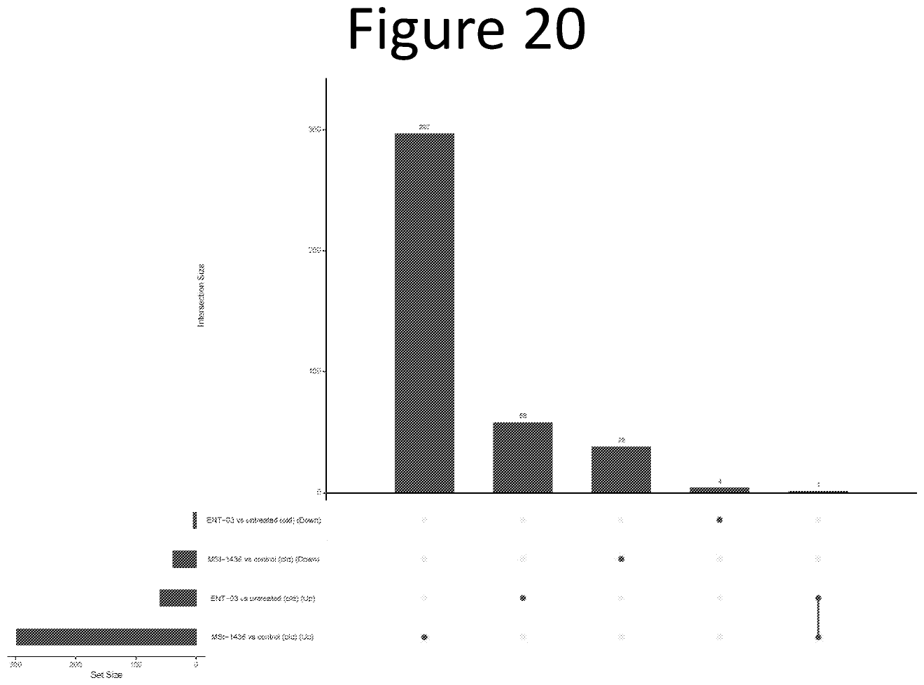

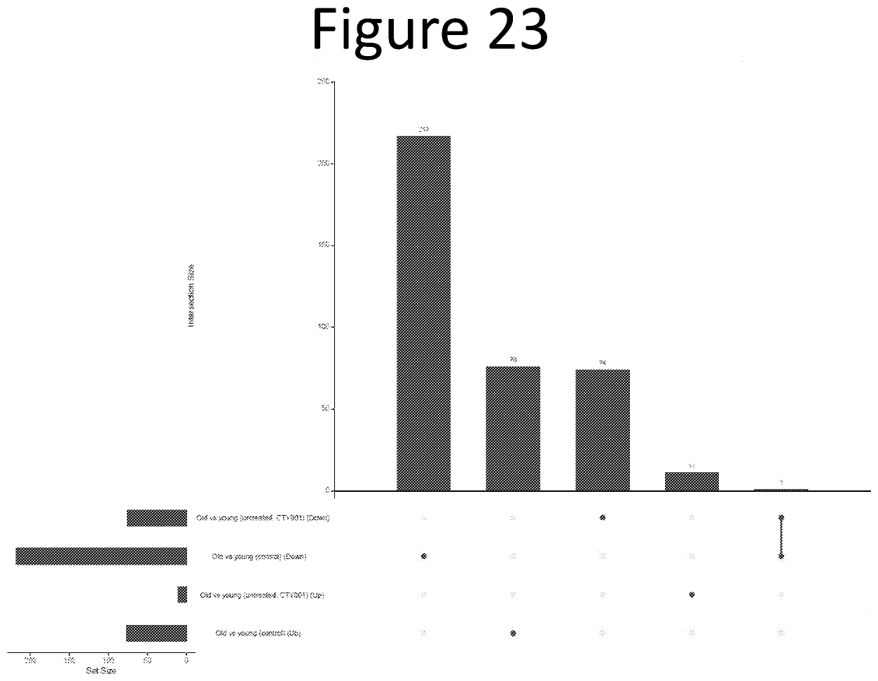

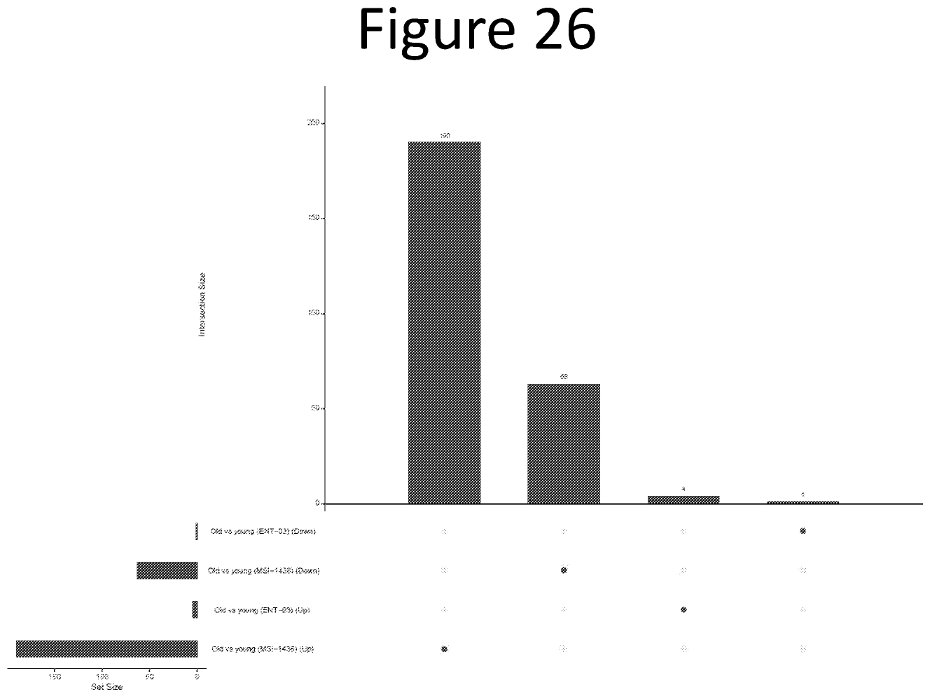

Scatter plots, upset plots, venn diagrams, hypergeometric tests and Spearman rank correlation tests were employed to assess the level of overlap as shown in FIGS. 15-27. Note that significantly differentially expressed genes in ENT-03-specific contrasts were determined using a statistical threshold of FDR-adjusted P<0.05. Whereas the ENT-02 (MSI-1436)-specific contrasts in this study defined significant genes using a cutoff of greater than four-fold change in expression.

### Genes Down-Regulated in Ageing and Up-Regulated Upon Treatment

Transcripts that were significantly down-regulated in ageing and up-regulated by treatment of old mice were identified for each compound. Congruence between these sets of genes was then assessed by comparing ENT-02 (MSI-1436)-affected genes to those of ENT-03, these results are illustrated in the Venn diagram in FIG. 15.

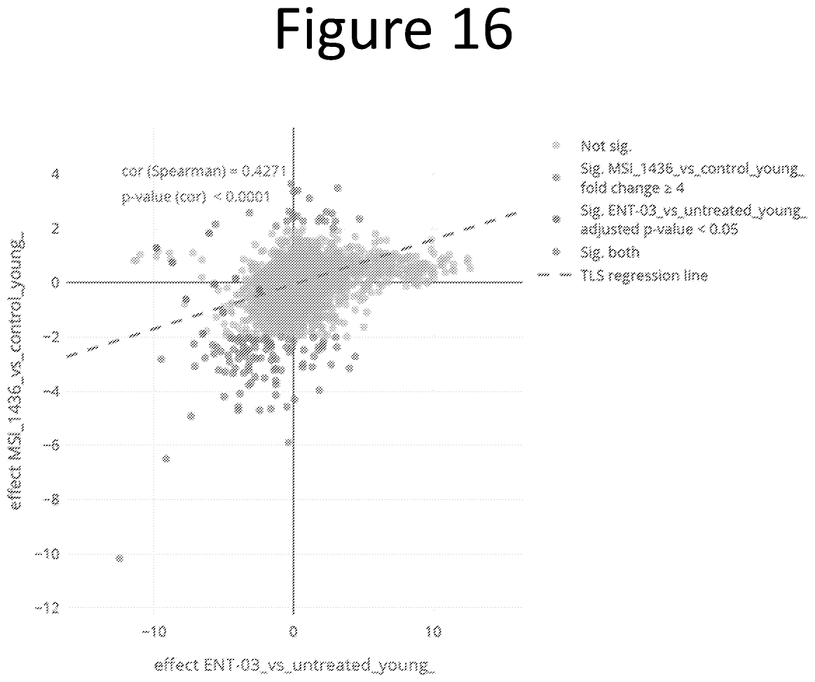

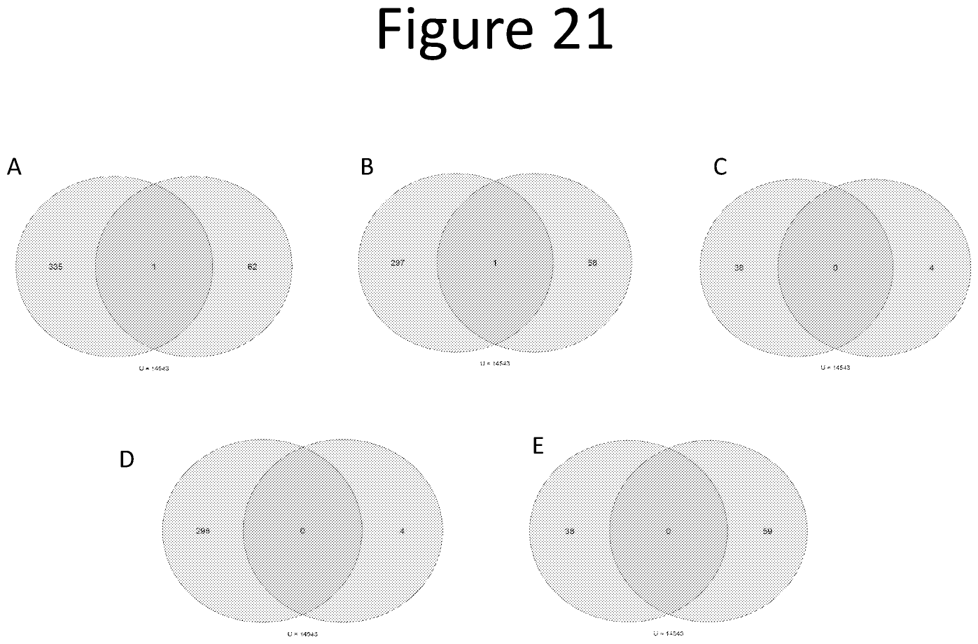

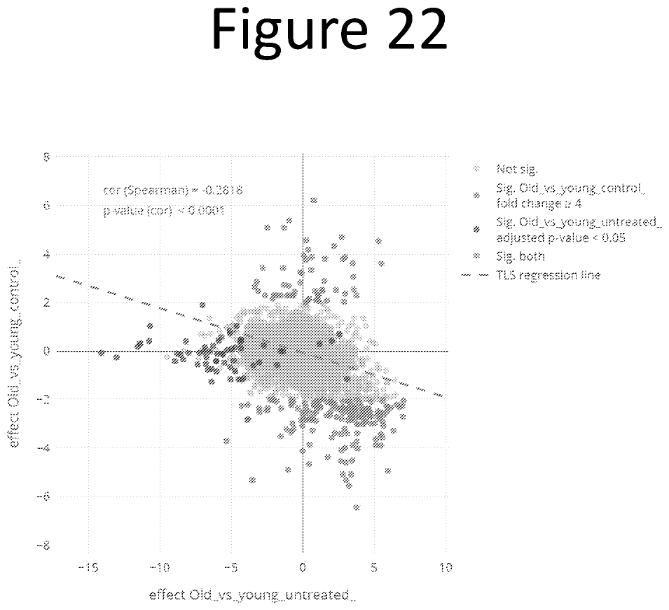

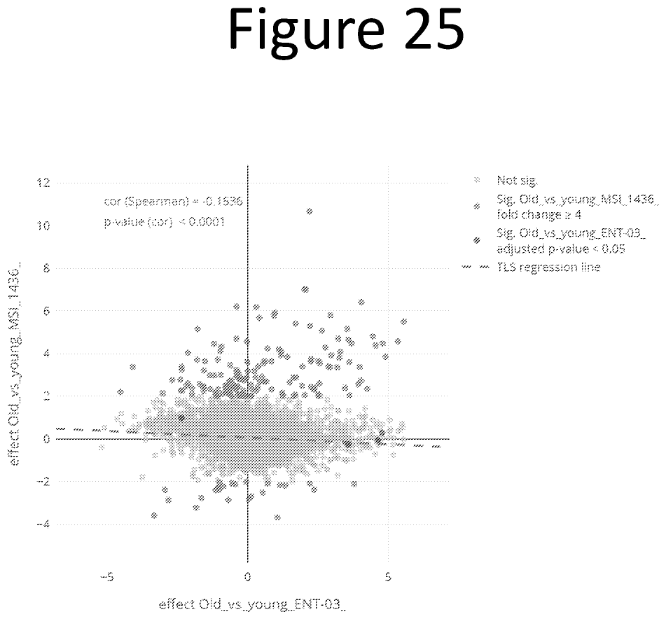

Congruence analysis results are shown in FIG. 16 which shows a scatter polot comparing significant genes in ENT-02 (MSI-1436) vs. control (young) against ENT-03 vs untreated (young). FIG. 22 shows a scatter plot comparing significant genes in Old vs young (control) against Old vs young (untreated). FIG. 25 shows a scatter plot comparing significant genes in Old vs young (ENT-02 (MSI-1436)) against Old vs young (ENT-03).

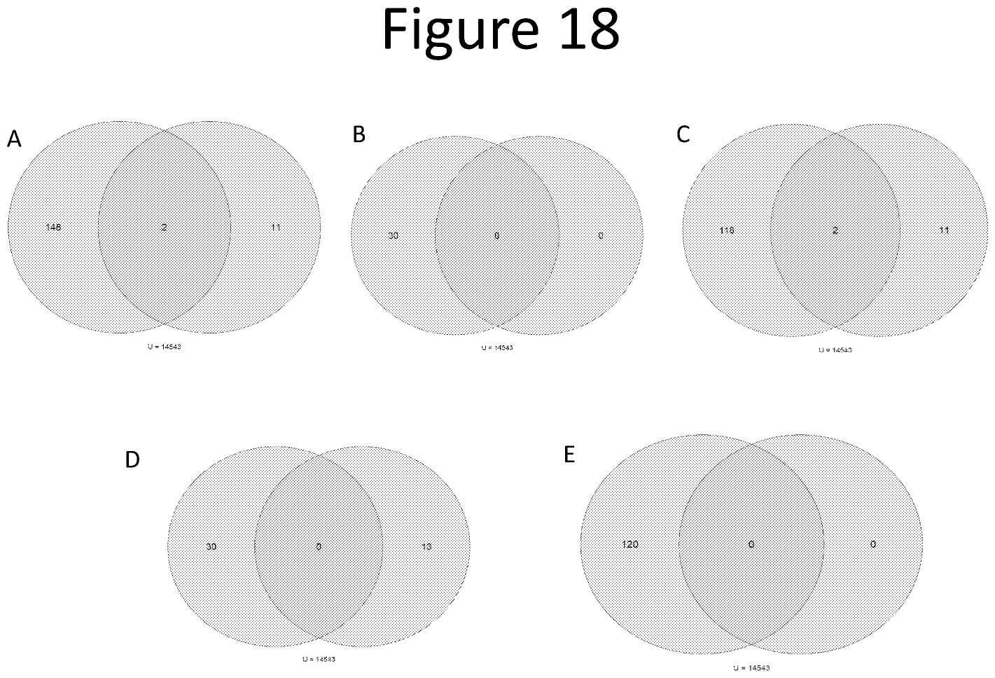

FIG. 18 shows venn diagrams of significant genes in ENT-02 (MSI-1436) vs control (young) against ENT-03 vs untreated (young). Each plot considers a different interaction of sets; either ignoring direction of perturbation, considering only up-regulated genes, considering only down-regulated genes, or examining the over-lap between those genes up-regulated in one contrast and those genes down-regulated in another. The symbol U denotes the universe.

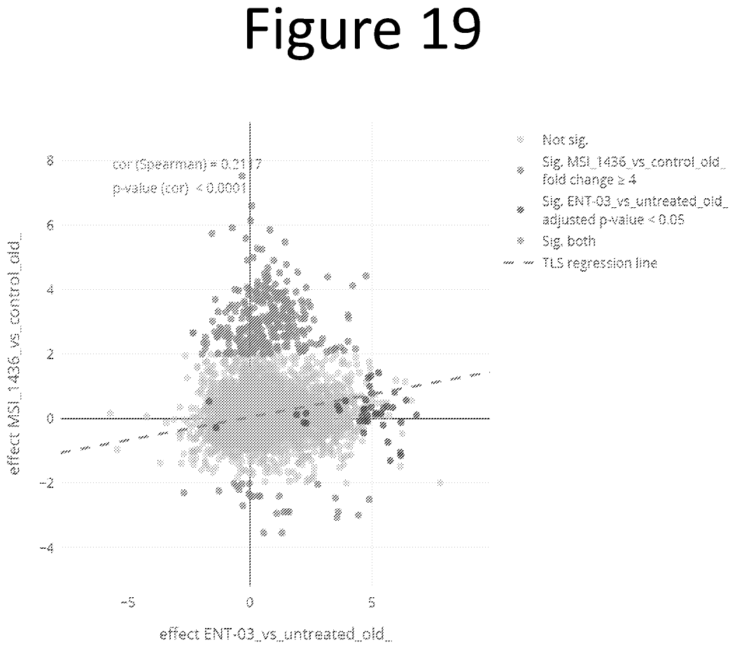

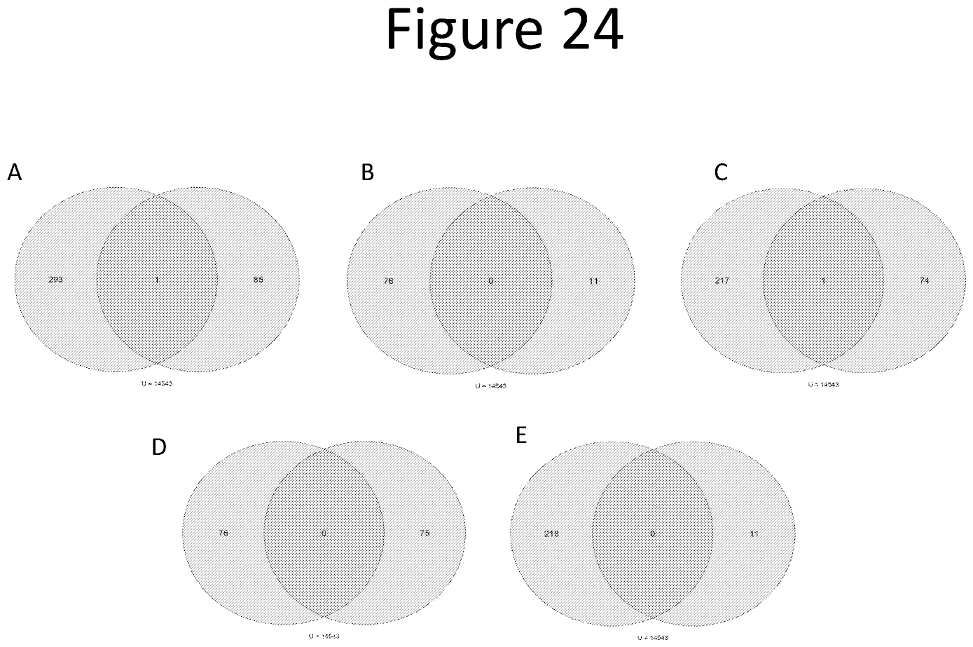

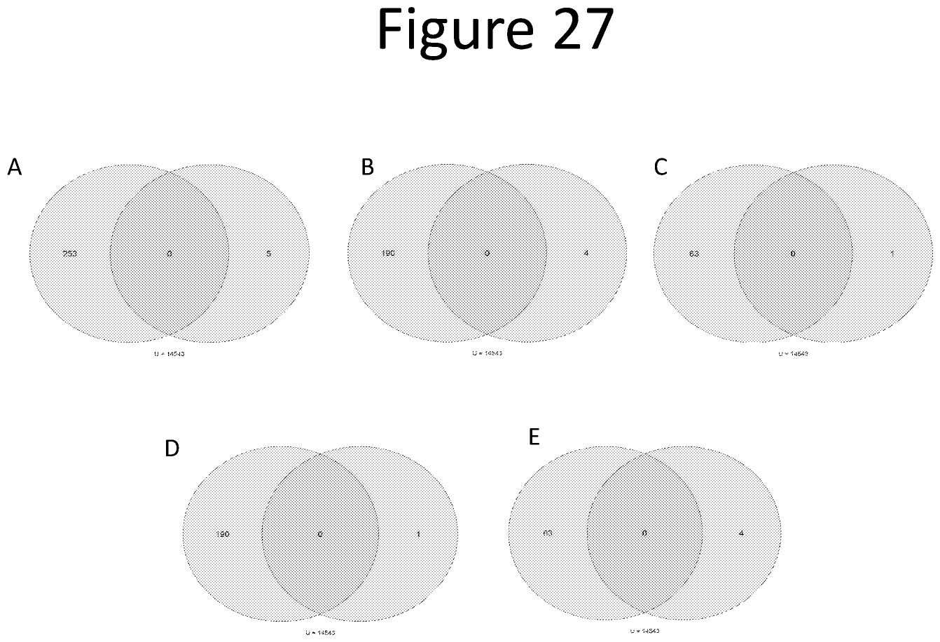

Congruence analysis results are shown in FIG. 19 which shows a scatter polot comparing significant genes in ENT-02 (MSI-1436) vs. control (old) against ENT-03 vs untreated (old). FIG. 21 shows Venn diagrams of significant genes in ENT-02 (MSI-1436) vs control (old) against ENT-03 vs untreated (old). FIG. 24 shows Venn diagrams of significant genes in Old vs young (control) against Old vs young (untreated). FIG. 27 shows Venn diagrams of significant genes in Old vs young (ENT-02 (MSI-1436)) against Old vs young (ENT-03). Venn diagrams of up- and down-regulated genes. Each plot considers a different interaction of sets; either ignoring direction of perturbation, considering only up-regulated genes, considering only down-regulated genes, or examining the overlap between those genes up-regulated in one contrast and those genes down-regulated in another. The symbol U denotes the universe.

### Example 11: Evaluation of a Deuterated ENT-03 Compound

ENT-03, a polyamine-bile acid conjugate, is expected to be metabolized through the chain of reactions utilized in the synthesis of bile acids. The following molecule was synthesized ENT-03D₃ (C24D₂, C25D₁)

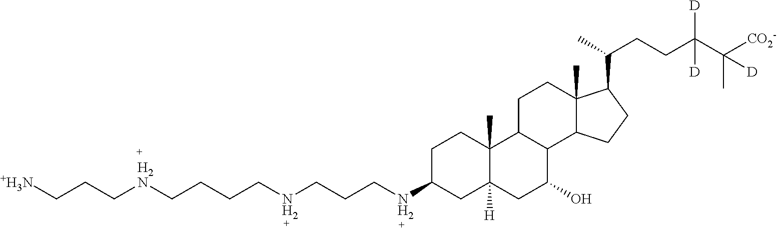

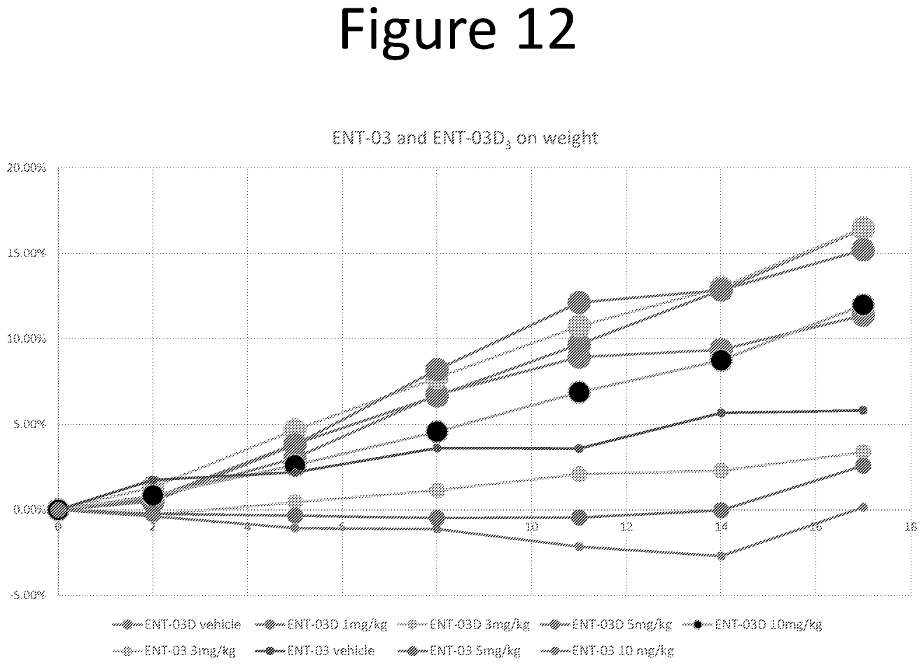

When administered i.p. to mice the animals gained weight, in contrast to the loss of weight observed when ENT-03 was administered. See FIG. 12. The pharmacological effect is best explained by the presence of the deuterium isotope on the kinetics of the enzymes that successively metabolize the cholesterol side chain, since the deuterated form of ENT-03 should exhibit the same pharmacology as ENT-03. The presence of the deuterium on the side chain likely slows the action of the 2-methylacyl-coenzyme A racemase on C-25, the subsequent action of the dehydrogenase that produces the 24,25-trans-unsaturated derivative by removing the deuterium atoms at C₂₄ and C₂₅, and the subsequent hydration and oxidation at the C24 bond catalyzed by the D-bifunctional protein. Instead, the metabolism of ENT-03D₃ will begin with the oxidation at C24 by CYP46A1, yielding the following compound:

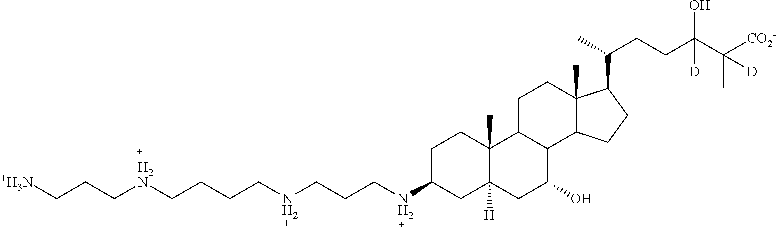

Since the pharmacological effect is weight gain, one can readily predict that the compound inhibits the PTPN11/SHP2 with highest specificity, since SHP2 is known to enhance the central leptin pathway. Inhibition of SHP2 would increase leptin resistance, which would result in an increase in appetite and accumulation of adipose tissue.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety. Further embodiments are set forth in the following claims.

### REFERENCES

Aarsland et al., “Neuropsychiatric symptoms in patients with Parkinson's disease and dementia: frequency, profile and associated care giver stress,” J. Neurol. Neurosurg. Psychiatry, 78:36-42 (2007).
Acsadi et al., “Alpha-synuclein loss in spinal muscular atrophy,” J. Mol. Neurosci., 43(3):275-83 (2011).
Ahima R S, Flier J S. “Leptin,” Annu Rev Physiol 62, 413-437 (2000).
Ahima R S, Patel H R, Takahashi N, Qi Y, Hileman S M, Zasloff M A. “Appetite suppression and weight reduction by a centrally active aminosterol,” Diabetes, 51(7):2099-104 (2002).
Akhter, S., S. K. Nath, et al. (1999). “Squalamine, a novel cationic steroid, specifically inhibits the brush-border Na+/H+ exchanger isoform NHE3.” Am J Physiol 276(1 Pt 1): C136-44.
Andresen, et al., “Effect of 5 Days Linaclotide on Transit and Bowel Function in Females With Constipation-Predominant Irritable Bowel Syndrome,” Gastroenterology, Volume 133, Issue 3, September 2007, Pages 761-768.
Antonelou et al., “Decreased levels of alpha-synuclein in cerebrospinal fluid of patients with clinically isolated syndrome and multiple sclerosis,” J. Neurochem., 134(4):748-55 (2015).
Antonio-Rubio, et al., “Abnormal thermography in Parkinson's disease,” Parkinsonism Relat Disord., 2015 August;21(8):852-7.
Auyeung et al., “Ten year survival and outcomes in a prospective cohort of new onset Chinese Parkinson's disease patients,” J. Neurol. Neurosurg. Psychiatry, 83:607-11 (2012).
Berg et al., “MDS Research Criteria for Prodromal Parkinson's Disease,” Mov. Disord., 30:1600-1611 (2015).
Beach et al. Neurol Ther. 2017 July; 6(Suppl 1): 5-13
Bhargava et al., “A phase I and pharmacokinetic study of squalamine, a novel antiangiogenic agent, in patients with advanced cancers,” Clin. Cancer Res., 7:3912-9 (2001).
Bjorkhem, “Five decades with oxysterols,” Biochimie 95, 448-454 (2013).
Boeve et al., “Synucleinopathy pathology and REM sleep behavior disorder plus dementia or parkinsonism,” Neurology 61(1):40-5 (July 2003).
A. E. Boniakowski et al., Murine macrophage chemokine receptor CCR2 plays a crucial role in macrophage recruitment and regulated inflammation in wound healing. Eur J Immunol 48, 1445-1455 (2018).
(a) Braak et al., “Idiopathic Parkinson's disease: possible routes by which vulnerable neuronal types may be subject to neuroinvasion by an unknown pathogen,” J. Neural. Transm. (Vienna), 110:517-36 (2003).
(b) Braak et al., “Staging of brain pathology related to sporadic Parkinson's disease,” Neurobiol. Aging, 24:197-211 (2003).
Breen, et al., “Sleep and circadian rhythm regulation in early Parkinson disease,” JAMA Neurol., 2014 May; 71(5):589-595.
Brundin, et al., “Prying into the Prion Hypothesis for Parkinson's Disease,” J Neurosci., 2017 Oct. 11; 37(41):9808-9818.
Brunel et al., “Squalamine: A Polyvalent Drug of the Future?” Current Cancer Drug Targets, 5(4):267-272(6) (2005)
Celotti, et al., “The 5 alpha-reductase in the brain: molecular aspects and relation to brain function,” Front Neuroendocrinol 13, 163-215 (1992).
Chen, et al., “Allosteric inhibition of SHP2 phosphatase inhibits cancers driven by receptor tyrosine kinases,” Nature 535, 148-152 (2016).
Claassen et al., “REM sleep behavior disorder preceding other aspects of synucleinopathies by up to half a century,” Neurology, 75(6):494-499 (August 2010).
Combs A P, “Recent advances in the discovery of competitive protein tyrosine phosphatase 1B inhibitors for the treatment of diabetes, obesity, and cancer” J. Med. Chem., 53(6):2333-44 (2010)
Connolly, et al., “Squalamine Lactate for Exudative Age-Related Macular Degeneration,” Ophthalmology Clinics of North America, 19, 381-391 (2006).
Corrochano et al., “α-synuclein levels modulate Huntington's disease in mice,” Hum. Mol. Genet., 21(3):485-94 (February 2012).
Courtney, et al., “CD45 functions as a signaling gatekeeper in T cells,” Sci Signal 12, (2019).
Crick et al., “The oxysterol and cholestenoic acid profile of mouse cerebrospinal fluid,” Steroids, 99, 172-177 (2015).
Demirel et al., “Decreased Expression of α-Synuclein, Nogo-A and UCH-L1 in Patients with Schizophrenia: A Preliminary Serum Study,” Psychiatry Invest., 14(3) (2017).
Diederich et al., “Hallucinations in Parkinson disease,” Nat. Rev. Neurol., 5:331-42 (2006).
Edlmann, et al., “Pathophysiology of chronic subdural haematoma: inflammation, angiogenesis and implications for pharmacotherapy,” J Neuroinflammation 14, 108 (2017).
Elchebly et al., “Increased insulin sensitivity and obesity resistance in mice lacking the protein tyrosine phosphatase-1B gene” Science. 1999 Mar. 5; 283(5407):1544-8.
Eswaran et al., “Crystal structures and inhibitor identification for PTPN5, PTPRR and PTPN7: a family of human MAPK-specific protein tyrosine phosphatases,” Biochem J, 395, 483-491 (2006).
Fahn S E R, Members of the UPDRS Development Committee. UNIFIED PARKINSON'S DISEASE RATING SCALE. Florham Park, N J: Macmillan Health Care Information (1987).
J. Garcia Fortanet et al., Allosteric Inhibition of SHP2: Identification of a Potent, Selective, and Orally Efficacious Phosphatase Inhibitor. J Med Chem 59, 7773-7782 (2016)
Frank, et al., “Psychometric validation of a constipation symptom assessment questionnaire,” Scand J Gastroenterol., 1999 September;34(9):870-7.
Frisinia, et al., “The neuropathological basis for depression in Parkinson's disease,” Parkinsonism Relat Disord., 2009 February; 15(2): 144-148.
Fukunaga, et al., “Role of MAP kinase in neurons,” Mol Neurobiol 16, 79-95 (1998).
Genaidy, et al., “Effect of Squalamine on Iris Neovascularization in Monkeys,” Retina, December 2002—Volume 22—Issue 6-p 772-778.
Gjerstad et al., “Excessive daytime sleepiness in Parkinson disease: is it the drugs or the disease?” Neurology, 67:853-8 (2006).
Goetz, et al., “Risk factors for nursing home placement in advanced Parkinson's disease,” Neurology, 43:2227-9 (1993).
Griffiths, et al., “Oxysterol research: a brief review,” Biochem Soc Trans 47, 517-526 (2019).
Hao et al., “A Phase I and pharmacokinetic study of squalamine, an aminosterol angiogenesis inhibitor,” Clin. Cancer Res., 9:2465-71 (2003).
Heaton, et al., “Defecation frequency and timing, and stool form in the general population: a prospective study,” Gut., 1992 June; 33(6): 818-824.
Herbst, Roy S. et al., “A Phase I/IIA Trial of Continuous Five-Day Infusion of Squalamine Lactate (MSI-1256F) Plus Carboplatin and Paclitaxel in Patients with Advanced Non-Small Cell Lung Cancer,” Clin. Cancer Res. 9(11) (2003).
Higgins, et al., “Regression of Retinopathy by Squalamine in a Mouse Model,” Pediatric Research, volume 56, pages 144-149 (2004).
Higgins, et al., “Squalamine Improves Retinal Neovascularization,” Investigative Ophthalmology & Visual Science, May 2000, Vol. 41, 1507-1512.
Hofmann, et al., “Bile salts of vertebrates: structural variation and possible evolutionary significance,” J Lipid Res 51, 226-246 (2010).
Holmqvist, et al., “Direct evidence of Parkinson pathology spread from the gastrointestinal tract to the brain in rats,” Acta neuropathological, 128. 10.1007/s00401-014-1343-6 (2014).
Hosokawa et al., “Accumulation of multiple neurodegenerative disease-related proteins in familial frontotemporal lobar degeneration associated with granulin mutation,” Scientific Reports, 7:1513 (2017).
S. Huber et al., IL-22BP is regulated by the inflammasome and modulates tumorigenesis in the intestine. Nature 491, 259-263 (2012).
Hughes, et al., “What features improve the accuracy of clinical diagnosis in Parkinson's disease,” Neurology, Jun. 1, 1992; 42 (6).
Javitt, “25R,26-Hydroxycholesterol revisited: synthesis, metabolism, and biologic roles,” J Lipid Res 43, 665-670 (2002).
Kim et al., “Poststroke Induction of α-Synuclein Mediates Ischemic Brain Damage,” J. Neurosci., 36(26):7055-65 (2016).
Kimura, et al., “Profile of urinary bile acids in infants and children: developmental pattern of excretion of unsaturated ketonic bile acids and 7beta-hydroxylated bile acids,” Pediatr Res 45, 603-609 (1999).
Krishnan, et al., “Targeting the disordered C terminus of PTP1B with an allosteric inhibitor,” Nat Chem Biol 10, 558-566 (2014).
Krishnan et al., “A potent, selective, and orally bioavailable inhibitor of the protein-tyrosine phosphatase PTP1B improves insulin and leptin signaling in animal models,” J. Biol. Chem., 293(5):1517-1525 (Feb. 2, 2018).
Kuo, et al., “Tyrosine phosphatases Shp1 and Shp2 have unique and opposing roles in oligodendrocyte development,” J Neurochem 113, 200-212 (2010).
Lantz, et al., “Inhibition of PTP1B by trodusquemine (MSI-1436) causes fat-specific weight loss in diet-induced obese mice,” Obesity (Silver Spring) 18, 1516-1523 (2010).
Lee, et al., “Protein tyrosine phosphatase-PEST and beta8 integrin regulate spatiotemporal patterns of RhoGDI1 activation in migrating cells,” Mol Cell Biol 35, 1401-1413 (2015).
Lewis, et al., “Stool form scale as a useful guide to intestinal transit time,”. Scand J Gastroenterol., 1997; 32:920-924.
S. M. Lewis, A. Williams, S. C. Eisenbarth, Structure and function of the immune system in the spleen. Sci Immunol 4, (2019).
Limbocker, et al., “Trodusquemine enhances Abeta42 aggregation but suppresses its toxicity by displacing oligomers from cell membranes,” Nat Commun 10, 225 (2019).
Lin, et al., “Genetics and genomics of Parkinson's disease,” Genome Medicine, 2014 6:48.
Liu, et al., “Nuclear hormone receptor regulation of microRNAs controls innate immune responses in C. elegans,” PLoS Pathog 9, e1003545 (2013).
Looney, et al., “Intracranial hemorrhage in asymptomatic neonates: prevalence on M R images and relationship to obstetric and neonatal risk factors,” Radiology 242, 535-541 (2007).
MacDonald, D. (1995). “Squalamine for STDs.” Abstract no F7 35th ICAAC conference.
Madrid-Navarro, et al., “Multidimensional Circadian Monitoring by Wearable Biosensors in Parkinson's Disease,” Front Neurol., 2018; 9: 157.
Magen et al., “Mouse Models of Cognitive Deficits Due to Alpha-Synuclein Pathology,” J. of Parkinson's Dis., 1:217-227 (2011).
Marquis, et al., “Development and validation of the Patient Assessment of Constipation Quality of Life questionnaire,” Scand J Gastroenterol., 2005 May; 40(5):540-51.
Mahanti, et al., “Comparative metabolomics reveals endogenous ligands of DAF-12, a nuclear hormone receptor, regulating C. elegans development and lifespan,” Cell Metab 19, 73-83 (2014).
McDowell et al., “Sleep dysfunction and EEG alterations in mice overexpressing alpha-synuclein,” J. Parkinsons Dis., 4(3):531-539 (2014).
McKhann, et al., “The diagnosis of dementia due to Alzheimer's disease: recommendations from the National Institute on Aging-Alzheimer's Association workgroups on diagnostic guidelines for Alzheimer's disease,” Alzheimers Dement., 2011 May; 7(3):263-9.
Meaney, et al., “Novel route for elimination of brain oxysterols across the blood-brain barrier: conversion into 7alpha-hydroxy-3-oxo-4-cholestenoic acid,” J Lipid Res 48, 944-951 (2007).
Mearin et al., “Bowel Disorders,” Gastroenterology, 150(6):1393-1407 (2016).
Moore, et al., “Squalamine: an aminosterol antibiotic from the shark,” Proceedings of the National Academy of Sciences of the United States of America 90, 1354-1358 (1993).
Morairty, “Detecting Neurodegenerative Diseases Before Damage Is Done,” SRI International (Jul. 26, 2013).
Y. Morita et al., Functional characterization of hematopoietic stem cells in the spleen. Exp Hematol 39, 351-359 e353 (2011).
Motola, et al., “Identification of ligands for DAF-12 that govern dauer formation and reproduction in C. elegans,” Cell 124, 1209-1223 (2006).
Moussaud et al., “Alpha-synuclein and tau: teammates in neurodegeneration?” Mol. Neurodeg., 9:43 (2014).
Mucke, et al., “High-level neuronal expression of abeta 1-42 in wild-type human amyloid protein precursor transgenic mice: synaptotoxicity without plaque formation,” J Neurosci 20, 4050-4058 (2000).
Mucke, et al., “High-Level Neuronal Expression of A?1-42 in Wild-Type Human Amyloid Protein Precursor Transgenic Mice: Synaptotoxicity without Plaque Formation,” The Journal of Neuroscience 20, 4050-4058 (2000).
Nagata, et al., “Changes in the level of 7 alpha-hydroxy-3-oxo-4-cholestenoic acid in cerebrospinal fluid after subarachnoid hemorrhage,” Neurol Med Chir (Tokyo) 35, 294-297 (1995).
Nagata, et al., “Identification of 7 alpha-hydroxy-3-oxo-4-cholestenoic acid in chronic subdural hematoma,” Biochim Biophys Acta 1126, 229-236 (1992).
Nakagawa, et al., “Bile acid metabolism in early life: studies of amniotic fluid,” J Lipid Res 31, 1089-1098 (1990).
Ogundare, et al., “Cerebrospinal fluid steroidomics: are bioactive bile acids present in brain?” J Biol Chem 285, 4666-4679 (2010).
Ondo et al., “Daytime sleepiness and other sleep disorders in Parkinson's disease,” Neurology, 57:1392-6 (2001).
Ondo, et al., “Placebo-controlled trial of lubiprostone for constipation associated with Parkinson disease,” Neurology, 2012 May 22; 78(21):1650-4.
Ortiz-Tudela et al., “Ambulatory circadian monitoring (ACM) based on thermometry, motor activity and body position (TAP): a comparison with polysomnography,” Physiol. Behav., 126:30-8 (2014).
Palma et al., “Treatment of autonomic dysfunction in Parkinson disease and other synucleinopathies”. Mov. Disord. (Review), 33(3):372-90 (March 2018).
Palsetia et al., “The Clock Drawing Test versus Mini-mental Status Examination as a Screening Tool for Dementia: A Clinical Comparison,” Indian J. Psychol. Med., 40:1-10 (2018).
Pandey, et al., “LMO4 is required to maintain hypothalamic insulin signaling,” Biochem Biophys Res Commun 450, 666-672 (2014).
Pandey, et al., “The LIM domain only 4 protein is a metabolic responsive inhibitor of protein tyrosine phosphatase 1B that controls hypothalamic leptin signaling,” J Neurosci 33, 12647-12655 (2013).
Papapetropoulos et al., “A questionnaire-based (UM-PDHQ) study of hallucinations in Parkinson's disease,” BMC Neurol., 8:21 (2008).
Pelkmans, L. and A. Helenius (2003). “Insider information: what viruses tell us about endocytosis.” Curr Opin Cell Biol 15(4): 414-22.
Perni, et al., “Multistep Inhibition of alpha-Synuclein Aggregation and Toxicity in Vitro and in Vivo by Trodusquemine,” ACS Chem Biol 13, 2308-2319 (2018).
Phillips, et al., “Alpha-synuclein-immunopositive myenteric neurons and vagal preganglionic terminals: autonomic pathway implicated in Parkinson's disease?,” Neuroscience. 2008 May 15; 153(3):733-50.
Pulido, et al., “PTPs emerge as PIPs: protein tyrosine phosphatases with lipid-phosphatase activities in human disease,” Hum Mol Genet 22, R66-76 (2013).
Qin, et al., “Chronic stress induces anxiety via an amygdalar intracellular cascade that impairs endocannabinoid signaling,” Neuron 85, 1319-1331 (2015).
Rao, et al., “Aminosterols from the dogfish shark Squalus acanthias,” J Nat Prod 63, 631-635 (2000).
Ricke, et al., “Neuronal Protein Tyrosine Phosphatase 1B Hastens Amyloid beta-Associated Alzheimer's Disease in Mice,” J Neurosci 40, 1581-1593 (2020).
Rocca et al., “The Role of T1-Weighted Derived Measures of Neurodegeneration for Assessing Disability Progression in Multiple Sclerosis,” Front Neurol., 8:433 (Sep. 4, 2017).
Saeed, et al., “7alpha-hydroxy-3-oxo-4-cholestenoic acid in cerebrospinal fluid reflects the integrity of the blood-brain barrier,” J Lipid Res 55, 313-318 (2014).
Saeed, et al., “Effects of a disrupted blood-brain barrier on cholesterol homeostasis in the brain,” J Biol Chem 289, 23712-23722 (2014).
Salmi et al., “Squalamine: An Appropriate Strategy against the Emergence of Multidrug Resistant Gram-Negative Bacteria?” PLoS One. 3(7):e2765 (2008).
Sarabia et al., “Circadian rhythm of wrist temperature in normal-living subjects A candidate of new index of the circadian system,” Physiol. Behav., 95:570-80 (2008).
Schiller, J H and G. Bittner, “Potentiation of platinum antitumor effects in human lung tumor xenografts by the angiogenesis inhibitor squalamine: effects on tumor neovascularization,” Clin Cancer Res. 5(12):4287-94 (1999).
Shehata et al., “Neuronal stimulation induces autophagy in hippocampal neurons that is involved in AMPA receptor degradation after chemical long-term depression,” J. Neurosci., 32:10413-22 (2012).
A. Shibuya et al., Fc alpha/mu receptor mediates endocytosis of IgM-coated microbes. Nat Immunol 1, 441-446 (2000).
Sills Jr., Allen K., et al., “Squalamine Inhibits Angiogenesis and Solid Tumor Growth in Vivo and Perturbs Embryonic Vasculature,” Cancer Research 58:2784-2792 (1998).
Smith, et al., “The protein tyrosine phosphatase 1B inhibitor MSI-1436 stimulates regeneration of heart and multiple other tissues,” NPJ Regen Med 2, 4 (2017).
Sokoloff, et al., “Adjunctive therapy for men with high risk localized and locally advanced prostate cancer: targeting disseminated tumor cells,” J Urol., 2004 December;172(6 Pt 2):2539-44.
R. Spolski, W. J. Leonard, Interleukin-21: a double-edged sword with therapeutic potential. Nat Rev Drug Discov 13, 379-395 (2014).
Sriwimol and Limprasert, “Significant Changes in Plasma Alpha-Synuclein and Beta-Synuclein Levels in Male Children with Autism Spectrum Disorder,” BioMed Research International, 2018, 7 pages (2018).
Steer et al., “Use of the Beck Depression Inventory-II with depressed geriatric inpatients,” Behav. Res. Ther., 38:311-8 (2000).
Stiasny-Kolster et al., “The REM sleep behavior disorder screening questionnaire—a new diagnostic instrument,” Movement disorders: Official J. of the Movement Dis. Soc., 22:2386-93 (2007).
Stoessl, “Neuroimaging in the early diagnosis of neurodegenerative disease,” Transl. Neurodegener., 1: 5 (2012).
U. Svajger, M. Anderluh, M. Jeras, N. Obermajer, C-type lectin DC-SIGN: an adhesion, signalling and antigen-uptake molecule that guides dendritic cells in immunity. Cell Signal 22, 1397-1405 (2010).
Svensson, et al., “Vagotomy and subsequent risk of Parkinson's disease,” Ann. Neurol., 2015; 78:522-529.
Takahashi, et al., “A novel aminosterol reverses diabetes and fatty liver disease in obese mice,” J Hepatol 41, 391-398 (2004).
The US Burden of Disease Collaborators, “The State of US Health, 1990-2016: Burden of Diseases, Injuries, and Risk Factors Among US States,” JAMA, 319(14):1444-1472 (2018)
Thompson, et al., “Pharmacological inhibition of protein tyrosine phosphatase 1B protects against atherosclerotic plaque formation in the LDLR(−/−) mouse model of atherosclerosis,” Clin Sci (Lond) 131, 2489-2501 (2017).
Videnovic, et al., “Circadian Dysregulation in Parkinson's Disease,” Neurobiol Sleep Circadian Rhythms, 2017 January;2:53-58.
Wahlen, et al., “Ketonic bile acids in urine of infants during the neonatal period,” J Lipid Res 30, 1847-1857 (1989).
Williams, J I et al., “Squalamine treatment of human tumors in nu/nu mice enhances platinum-based chemotherapies,” Clin Cancer Res. (2001).
Wiesmann, C et al., 2004. Nat. Struct. Mol. Biol. 11, 730.
Wimo, et al., “The worldwide economic impact of dementia 2010,” Alzheimer's Dement., 9: 1-11 (2013).
F. M. Wolber et al., Roles of spleen and liver in development of the murine hematopoietic system. Exp Hematol 30, 1010-1019 (2002).
Yamashima, “The inner membrane of chronic subdural hematomas: pathology and pathophysiology,” Neurosurg Clin N Am 11, 413-424 (2000).
Yancopoulou et al., “Tau and alpha-synuclein inclusions in a case of familial frontotemporal dementia and progressive aphasia,” J. Neuropathol. Exp. Neurol., 64(3):245-53 (2005).
Yin, M., C. Gentili, et al. (2002). “Antiangiogenic treatment delays chondrocyte maturation and bone formation during limb skeletogenesis,” J. Bone Miner Res 17(1):56-65.
Yoshiyama, et al., “Synapse loss and microglial activation precede tangles in a P301S tauopathy mouse model,” Neuron 53, 337-351 (2007).
Zabolotny, et al., “PTP1B regulates leptin signal transduction in vivo,” Dev Cell 2, 489-495 (2002).
Zahodne, et al., “Components of Depression in Parkinson Disease,” J Geriatr Psychiatry Neurol., 2012 September; 25(3): 131-137.
Zasloff, et al., “A spermine-coupled cholesterol metabolite from the shark with potent appetite suppressant and antidiabetic properties,” Int J Obes Relat Metab Disord., 2001 May; 25(5):689-97.
Zasloff, et al., “A spermine-coupled cholesterol metabolite from the shark with potent appetite suppressant and antidiabetic properties,” Int J Obes Relat Metab Disord 25, 689-697 (2001).
Zhang, et al., “Metabolism of 27-, 25- and 24-hydroxycholesterol in rat glial cells and neurons,” Biochem J 322 (Pt 1), 175-184 (1997).
Zhang, et al., “Neuronal Shp2 tyrosine phosphatase controls energy balance and metabolism,” Proceedings of the National Academy of Sciences of the United States of America 101, 16064-16069 (2004).
Zheng, et al., “Gain-of-function mutations in the gene encoding the tyrosine phosphatase SHP2 induce hydrocephalus in a catalytically dependent manner,” Sci Signal 11, (2018).
Zhao et al., “A comparative study of the amount of α-synuclein in ischemic stroke and Parkinson's disease,” Neurol. Sci., 37(5):749-54 (2016).
Zinsmeister et al., “Pharmacodynamic and clinical endpoints for functional colonic disorders: statistical considerations,” Dig. Dis. Sci., 58:509-18 (2013).

1. An aminosterol compound selected from the group consisting of;
(a) a compound having Formula I-R: 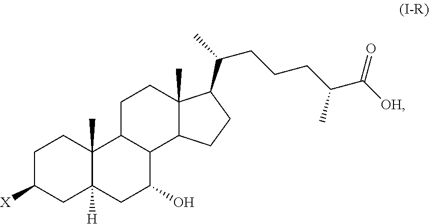
or a pharmaceutically acceptable salt, solvate, prodrug, or derivative thereof, wherein X is: 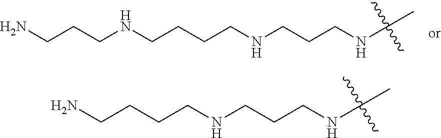 [SMILES: *NCCCNCCCCNCCCN ; *NCCCNCCCCN]
(b) a compound having Formula I-S: 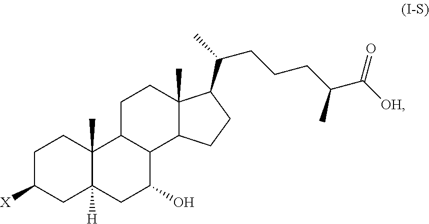
or a pharmaceutically acceptable salt. solvate, prodrug, or derivative thereof, wherein X is: 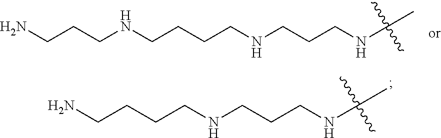 [SMILES: *NCCCNCCCCNCCCN ; *NCCCNCCCCN]
(c) a compound having the formula of Compound I-R: 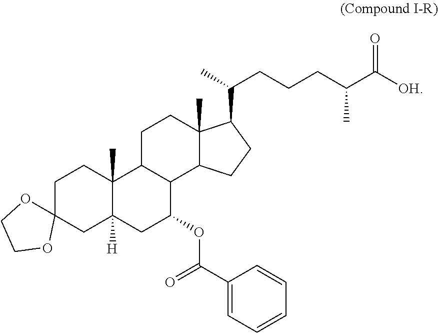 [SMILES: C[C@H](CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]
or a pharmaceutically acceptable salt, solvate, or derivative thereof; and
(d) a compound having the formula of Compound I-S: 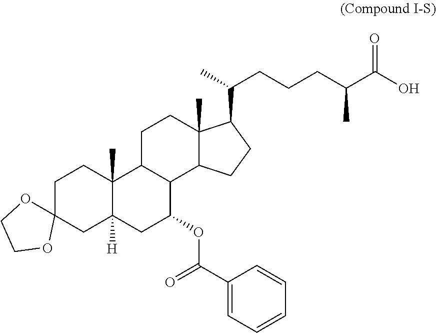 [SMILES: C[C@H](CCC[C@H](C)C(=O)O)[C@H]1CCC2C3C(CC[C@@]21C)[C@@]1(C)CCC2(C[C@@H]1C[C@H]3OC(=O)c1ccccc1)OCCO2]
or a pharmaceutically acceptable salt, solvate, or derivative thereof.

2. (canceled)

3. The aminosterol compound:
(a) of claim 1(a):
(i) having the formula of Compound III-R: 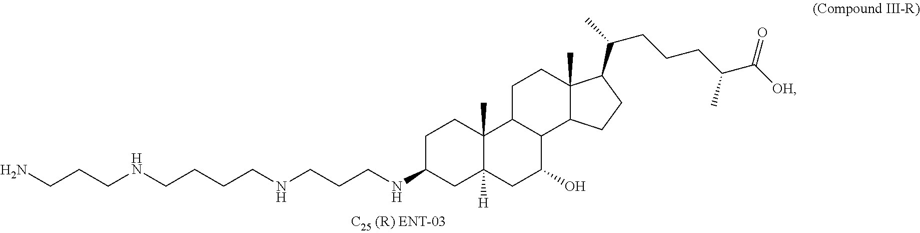 [SMILES: C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)O]
or a pharmaceutically acceptable salt, solvate, prodrug, or derivative thereof; or
(ii) having the formula of Compound VI-R: 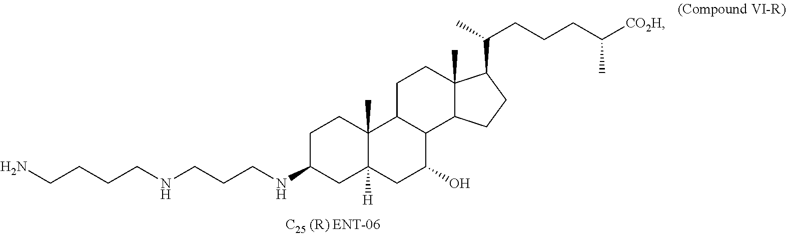 [SMILES: C[C@H](CCC[C@@H](C)[C@H]1CCC2C3C(CC[C@@]21C)[C@@]1(C)CC[C@H](NCCCNCCCCN)C[C@@H]1C[C@H]3O)C(=O)O]
or a pharmaceutically acceptable salt, solvate, prodrug, or derivative thereof; or
(b) of claim 1(b):
(i) having the formula of Compound III-S: 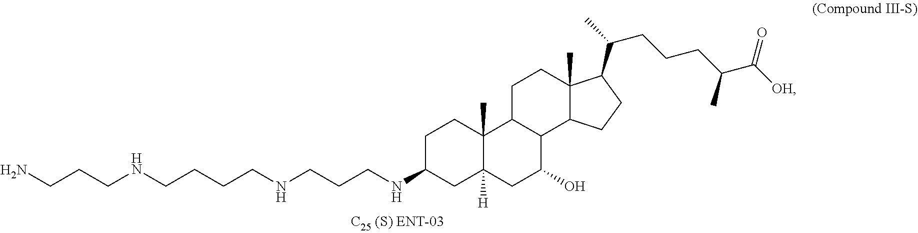 [SMILES: C[C@H](CCC[C@H](C)C(=O)O)[C@H]1CCC2C3C(CC[C@@]21C)[C@@]1(C)CC[C@H](NCCCNCCCCNCCCN)C[C@@H]1C[C@H]3O]
or a pharmaceutically acceptable salt, solvate, prodrug, or derivative thereof or
(ii) having the formula of Compound VI-S: 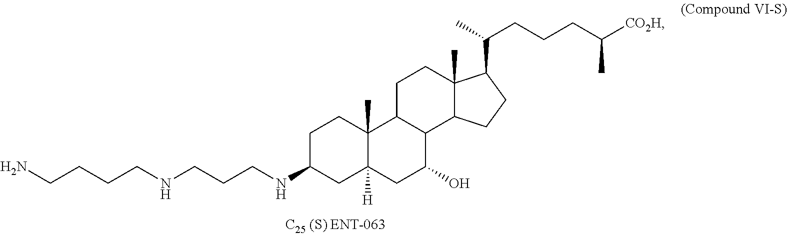 [SMILES: C[C@H](CCC[C@H](C)C(=O)O)[C@H]1CCC2C3C(CC[C@@]21C)[C@@]1(C)CC[C@H](NCCCNCCCCN)C[C@@H]1C[C@H]3O]
or a pharmaceutically acceptable salt, solvate, prodrug, or derivative thereof.

4.-8. (canceled)

9. The aminosterol compound of claim 1:
(a) which is the hydrochloride salt; or
(b) which is a sodium salt; and/or
(c) wherein the diastereomeric excess (de), with respect to the C₂₅ carbon is about 80% to about 90%, about 90% to about 95%, about 95% to about 99%, about 99% to about 99.9%, or about 100%.

10. (canceled)

11. A composition comprising an aminosterol compound according to claim 1, and at least one pharmaceutically acceptable carrier or excipient, and optionally wherein:
(a) the composition comprises one or more of the following: an aqueous carrier, a buffer, a sugar; and/or a polyol compound; and/or
(b) the composition further comprises at least one additional active agent.

12. (canceled)

13. The composition of claim 11, wherein the composition is formulated:
(a) for administration selected from the group consisting of oral, pulmonary, rectal, colonic, parenteral, intracisternal, intravaginal, intraperitoneal, intravenous, subcutaneous, intramuscular, nebulization, inhalation, ocular, otic, local, buccal, nasal, and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, and mixed immediate release and controlled release formulations;
(d) for oral administration;
(e) as an oral tablet or capsule;
(f) for intranasal administration; or
(g) any combination of (a), (b), (c), (d), (e), and (f).

14. A method of treating a subject in need having a condition susceptible to treatment with an aminosterol, comprising administering to the subject a therapeutically effective amount of an aminosterol compound according to claim 1; or
a method of treating, preventing, and/or slowing the onset or progression of a condition or disorder, or a related symptom, correlated with abnormal alpha-synuclein pathology and/or dopaminergic dysfunction, in a subject in need, comprising administering a therapeutically effective amount of an aminosterol compound according to claim 1.

15. The method of claim 14, wherein:
(a) the symptom is selected from the group consisting of constipation, hallucinations, cognitive impairment, and inflammation;
(b) the symptom is correlated with a synucleopathy, a neurodegenerative disease, a neurological disease or disorder, a psychological and/or behavior disorder, or a cerebral or general ischemic disorder or condition; or
(c) the condition or disorder is a synucleopathy, neurodegenerative disease, or neurological disease or disorder;
(d) the condition or disorder is a psychological and/or behavior disorder; or
(e) the condition or disorder is a cerebral or general ischemic disorder or condition.

16. The method of claim 15, wherein:
(a) the synucleopathy, neurodegenerative disease, or neurological disease or disorder is selected from the group consisting of Parkinson's disease, Alzheimer's disease, schizophrenia, multiple system atrophy, Lewy body dementia, dementia with Lewy bodies, Huntington's Disease, Multiple Sclerosis, Amyotrophic Lateral Sclerosis, Friedreich's ataxia, vascular dementia, spinal muscular atrophy, supranuclear palsy, progressive nuclear palsy, frontotemporal dementia, progressive nuclear palsy, Guadeloupian Parkinsonism, spinocerebellar ataxia, parkinsonism, traumatic brain injury, degenerative processes associated with aging, and dementia of aging;
(b) the psychological or behavior disorder is selected from the group consisting of depression, autism, autism spectrum disorder, down syndrome, Gaucher's disease, Krabbe's disease, lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, major depressive disorder, and sleep disorders such as REM sleep behavior disorder (RBD), sleep fragmentation, REM behavior disorder, circadian rhythm dysfunction, sleep apnea, and cognitive impairment; or
(c) the cerebral or general ischemic disorder or condition is selected from the group consisting of microangiopathy, intrapartum, cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects, high blood pressure, low blood pressure, and pulmonary edema.

17. A method of treating, preventing, and/or slowing the onset or progression a cerebral or general ischemic disorder and/or a related symptom, correlated with abnormal alpha-synuclein pathology and/or dopaminergic dysfunction, in a subject in need, comprising administering a therapeutically effective amount of an aminosterol compound according to claim 1, and
optionally wherein the cerebral or general ischemic disorder and/or a related symptom is selected from the group consisting of microangiopathy, intrapartum cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, low blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects (CCDs) and/or a related symptom, and pulmonary edema.

18. (canceled)

19. A method of suppressing, preventing and/or slowing the onset or progression of appetite or weight gain, and/or one or more related symptoms, in a subject in need thereof, comprising administering to the subject a therapeutically effective amount of an aminosterol compound according to claim 1.

20. The method of claim 14, wherein:
(a) the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof, and/or
(b) the method of administration is nasal administration, oral administration, or a combination thereof; and/or
(c) the therapeutically effective amount of the aminosterol compound or a pharmaceutically acceptable salt, solvate, prodrug, or derivative thereof comprises:
(i) about 0.1 to about 20 mg/kg body weight of the subject:
(ii) about 0.1 to about 15 mg/kg body weight of the subject;
(iii) about 0.1 to about 10 mg/kg body weight of the subject;
(iv) about 0.1 to about 5 mg/kg body weight of the subject;
(v) about 0.1 to about 2.5 mg/kg body weight of the subject;
(vi) about 0.001 to about 500 mg/day:
(vii) about 0.001 to about 250 mg/day:
(viii) about 0.001 to about 125 mg/day;
(ix) about 0.001 to about 50 mg/day:
(x) about 0.001 to about 25 mg/day:
(xi) about 0.001 to about 10 mg/day;
(xii) about 0.001 to about 6 mg/day;
(xiii) about 0.001 to about 4 mg/day; or
(xiv) about 0.001 to about 2 mg/day.

21. (canceled)

22. The method of claim 14, wherein:
(a) the method of administration comprises oral administration and wherein the therapeutically effective amount of the aminosterol compound or a pharmaceutically acceptable salt, solvate, prodrug, or derivative thereof comprises:
(i) about 1 to about 300 mg/day; or
(ii) about 25 to about 500 mg/day; and/or
(b) the aminosterol compound or a pharmaceutically acceptable salt, solvate, prodrug, or derivative thereof is administered in combination with at least one additional active agent to achieve either an additive or synergistic effect; and/or
(c) administration of the composition comprises administration on an empty stomach, optionally within two hours of the subject waking; and/or
(d) no food is consumed by the subject after about 60 to about 90 minutes from administration of the composition; and/or
(e) the aminosterol, or a pharmaceutically acceptable salt, solvate, prodrug, or derivative thereof, is of pharmaceutically acceptable grade; and/or
(f) a hydrochloride salt of the aminosterol is administered; and/or
(g) the subject is a human.

23.-25. (canceled)

26. The method of claim 14, further comprising:
(a) determining a dosage of the aminosterol or a pharmaceutically acceptable salt, solvate, prodrug, or derivative for the subject, wherein the aminosterol dosage is determined based on the effectiveness of the aminosterol dosage in improving or resolving a symptom being evaluated,
(b) followed by administering a composition comprising the dosage of the aminosterol to the subject for a period of time, wherein the method comprises:
(i) identifying a symptom to be evaluated, wherein the symptom is susceptible to treatment with an aminosterol;
(ii) identifying a starting dosage of an aminosterol thereof for the subject;
(iii) administering an escalating dosage of the aminosterol to the subject over a period of time until an effective dosage for the symptom being evaluated is identified, wherein the effective dosage is the aminosterol dosage where improvement or resolution of the symptom is observed, and fixing the aminosterol dosage at that level for that particular symptom in that particular subject.

27. The method of claim 26, wherein:
(a) the composition is administered orally and:
(i) the starting aminosterol dosage ranges from about 10 mg up to about 150 mg/day;
(ii) the dosage of the aminosterol for the subject following escalation is fixed at a range of from about 25 mg up to about 500 mg/day; and/or
(iii) the dosage of the aminosterol or a salt or derivative thereof is escalated in about 25 mg increments; or
(b) the composition is administered intranasally and:
(i) the starting aminosterol dosage ranges from about 0.001 mg to about 3 mg/day;
(ii) the dosage of the aminosterol for the subject following escalation is fixed at a range of from about 0.001 mg up to about 6 mg/day;
(iii) the dosage of the aminosterol for the subject following escalation is a dosage which is subtherapeutic when given orally or by injection; and/or
(iv) the dosage of the aminosterol is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg; and:
(c) optionally wherein
(i) the dosage of the aminosterol is escalated every about 3 to about 5 days; and/or
(ii) the starting aminosterol dosage is higher if the symptom being evaluated is severe; and/or
(iii) the symptom is correlated with abnormal alpha-synuclein pathology and/or dopaminergic dysfunction.

28. (canceled)

29. A method of producing an aminosterol compound:
(a) of Formula I-R, or a pharmaceutically acceptable salt, solvate, prodrug, or derivative thereof: 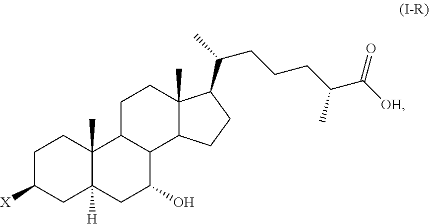
wherein X is 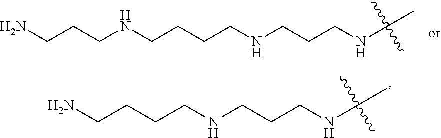 [SMILES: *NCCCNCCCCNCCCN ; *NCCCNCCCCN]
comprising asymmetric hydrogenation of Compound II: 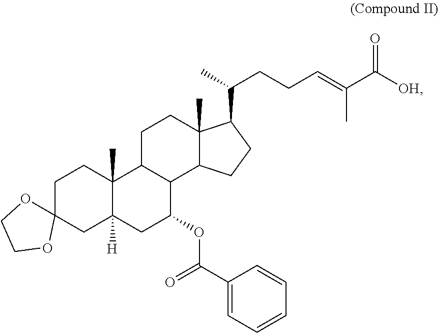 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O]
to produce Compound I-R: 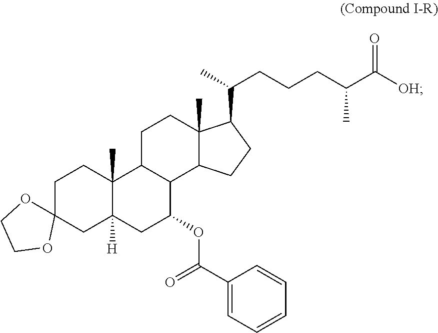 [SMILES: C[C@H](CCC[C@@H](C)[C@H]1CCC2C3C(CC[C@@]21C)[C@@]1(C)CCC2(C[C@@H]1C[C@H]3OC(=O)c1ccccc1)OCCO2)C(=O)O]
or
(b) of Formula I-S, or a pharmaceutically acceptable salt, solvate, prodrug, or derivative thereof: 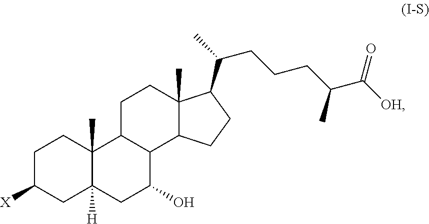
wherein X is 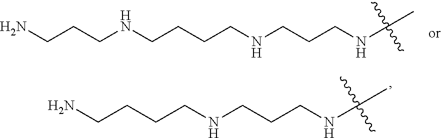 [SMILES: *NCCCNCCCCNCCCN ; *NCCCNCCCCN]
comprising asymmetric hydrogenation of Compound II: 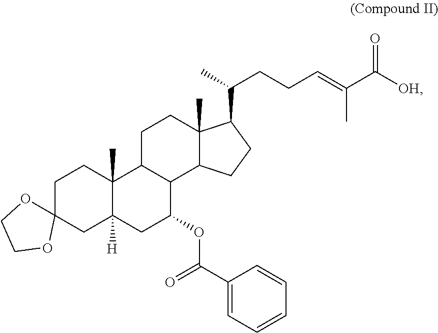 [SMILES: C/C(=C\CC[C@@H](C)[C@H]1CCC2C3C(CC[C@@]21C)[C@@]1(C)CCC2(C[C@@H]1C[C@H]3OC(=O)c1ccccc1)OCCO2)C(=O)O]
to produce Compound I-S:

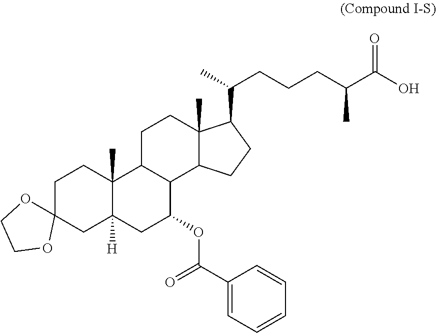 [SMILES: C[C@H](CCC[C@H](C)C(=O)O)[C@H]1CCC2C3C(CC[C@@]21C)[C@@]1(C)CCC2(C[C@@H]1C[C@H]3OC(=O)c1ccccc1)OCCO2]

30. (canceled)

31. The method of claim 29, wherein:
(a) X is 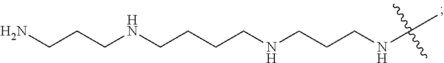 [SMILES: *NCCCNCCCCNCCCN]
or
(b) X is 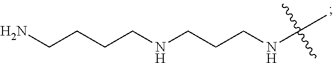 [SMILES: *NCCCNCCCCN]
and/or
(c) further comprising hydrolysis of the dioxolane of Compound I-R or Compound I-S to form a ketone and reductive amination of the ketone with amine: 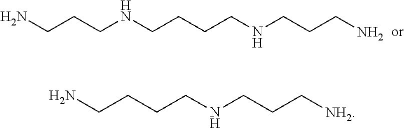 [SMILES: NCCCNCCCCNCCCN ; NCCCCNCCCN]
and/or
(d) asymmetric hydrogenation comprises mixing Compound IV: 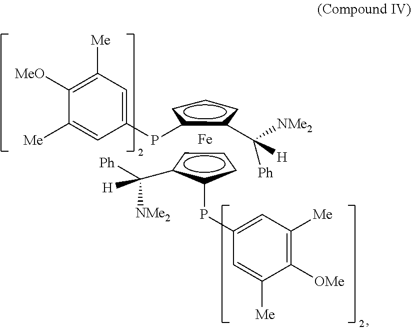
(Me-Allyl)2Ru(COD), and an acid to form a solution, and contacting Compound II with the solution and hydrogen (H₂); and/or
(e) asymmetric hydrogenation comprises mixing Compound V: 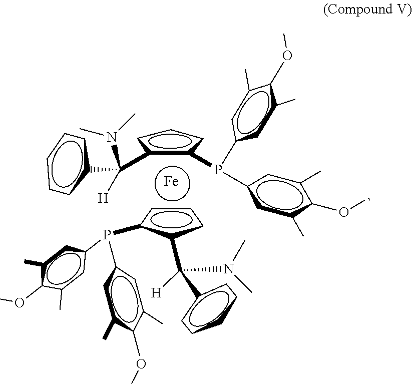
(Me-Allyl)2Ru(COD), and an acid to form a solution, and contacting Compound II with the solution and hydrogen (H₂).

32.-35. (canceled)

36. The method of claim 31(d) or 31(e), wherein:
(a) the acid comprises HBr; and/or
(b) the solution further comprises isopropyl acetate.

37. (canceled)

38. The method of claim 29, wherein:
(a) the asymmetric hydrogenation comprises subjecting Compound II to about 60 psi to about 70 psi, about 70 psi to about 80 psi, about 80 psi to about 90 psi, about 90 psi to about 100 psi, about 100 psi to about 120 psi, about 120 psi to about 140 psi, about 140 psi to about 160 psi, about 160 psi to about 180 psi, about 180 psi to about 200 psi, about 200 psi to about 250 psi, about 250 psi to about 300 psi, about 300 psi to about 350 psi, about 350 psi to about 400 psi, about 400 psi to about 500 psi, about 500 psi to about 550 psi, about 550 psi to about 600 psi, about 600 psi to about 650 psi, or about greater than about 650 psi, of hydrogen (H₂); and/or
(b) the asymmetric hydrogenation is at a temperature of about 20° C. to about 30° C., about 30° C. to about 40° C., about 40° C. to about 50° C., about 50° C. to about 60° C., or about greater than 60° C.; and/or
(c) the asymmetric hydrogenation is for about 12 hr to about 24 hr, about 24 hr to about 36 hr, about 36 hr to about 48 hr, about 48 hr to about 60 hr, or greater than about 60 hr; and/or
(d) the mol % s of Compound IV or Compound V, and (Me-Allyl)2Ru(COD) are about 0.2% to about 1%, about 1% to about 3%, about 3% to about 4%, about 4% to about 5%, about 5% to about 6%, or about 6% to about 8%, relative to Compound II; and optionally wherein the mol % of the acid is about 4% to about 6%, about 6% to about 8%, about 8% to about 10%, about 10% to about 12%, or about 12% to about 16%, relative to Compound II; and/or
(e) the diastereomeric excess (de) of the C₂₅ carbon is about 80% to about 90%, about 90% to about 95%, about 95% to about 99%, about 99% to about 99.9%, or about 100%.

39.-42. (canceled)
